SEQUENCE LISTING (1 sequence)

SEQ ID NO: 1 - DNA, 13 bp, Artificial Sequence
  Description of Artificial SequenceDNA sequence bound by peroxisome proliferator activated receptor alpha (PPARalpha)-RXR transcription factor heterodimer
aggtcanaggtca

(19) United States
(12) Patent Application Publication
(10) Pub. No.: US 2009/0082435 A1
(43) Pub. Date: Mar. 26, 2009

(54) Methods, Compositions, And Compounds For Modulation Of Monoacylglycerol Lipase, Pain, And Stress-Related Disorders

(75) Inventors: Daniele Piomelli, Irvine, CA (US); Andrea Duranti, Urbino (IT); Andrea Tontini, Pesaro (IT); Marco Mor, Ghedi (IT); Giorgio Tarzia, Petriano (IS); Andrea Hohmann, Athens, GA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: The Regents of the University of California, Oakland, CA (US); The University of Georgia Research, Athens, GA (US); Universita Degli Studi Di Urbino, Urbino (IT); Univestia Degli Studi Di Parma, Parma (IT)

(21) Appl. No.: 11/912,386

(22) PCT Filed: Apr. 26, 2006
(86) PCT No.: PCT/US2006/016843

§ 371 Date: Jul. 29, 2008

Related U.S. Application Data
(60) Provisional application No. 60/676,532, filed on Apr. 28, 2005.

(51) Int. Cl. A61K 31/27; C12Q 1/44; C07C 271/02; C12N 9/18; A61P 29/00

(52) U.S. Cl. 514/488; 435/19; 560/115; 435/198

(57) ABSTRACT

Methods, compositions, and compounds for inhibiting monoacyglycerol lipase, and for treating pain, for modulating stress-induced analgesia or for treating stress-induced disorders in mammals are provided.

### STATEMENT AS TO RIGHTS TO INVENTIONS MADE UNDER FEDERALLY SPONSORED RESEARCH AND DEVELOPMENT

This research was supported in part by grants from the National Institute on Drug Abuse DA 12413 and DA 12653. The government may have certain rights in the invention.

### REFERENCE TO A “SEQUENCE LISTING,” A TABLE, OR A COMPUTER PROGRAM LISTING APPENDIX SUBMITTED ON A COMPACT DISK

Not Applicable

### BACKGROUND OF THE INVENTION

Acute stress activates neural systems in the mammalian brain that inhibit pain sensation. This adaptive response depends on the recruitment of descending neural pathways, which from the amygdala project to the midbrain periaqueductal gray (PAG), the brainstem rostroventromedial medulla (RVM), and the spinal cord (Millan, M. J. Prog. Neurobiol. 66:355-474 (2002)). Endogenous opioid peptides are known to play a key role in this process (Lewis, J. W. et al., Science 208:623-625 (1980); Lewis, J. W. et al., Science 217:557-559 (1982); Grau, J. W. et al., Science 213:1409-1411 (1981); Akil, H. et al., Annals of the New York Academy of Sciences 467:140-153 (1986)). Nevertheless, the fact that opiate antagonists only block certain forms of stress analgesia—for example, that evoked in rats by intermittent foot shock (Lewis, J. W. et al., Science 208:623-625 (1980))—implies that other as-yet-unidentified neurotransmitter systems also must be involved. Identifying the endogenous mediators of nonopioid stress analgesia would be important both to elucidate the mechanisms by which stress regulates pain and to uncover novel therapeutic targets. The endocannabinoid system is suspected of being involved in stress analgesia for three reasons. First, agonists of CB₁ receptors—the predominant cannabinoid receptor subtype present in the central nervous system (Herkenham, M. et al. J. Neurosci. 11:563-583 (1991); Zimmer, A. et al., Proc. Natl. Acad. Sci. USA 96:5780-5785 (1999))—exert profound antinociceptive effects in animals, including humans (Walker, J. M. et al., Cannabinoids—Handbook of Experimental Pharmacology (ed. Pertwee, R.) 494-539 (Springer-Verlag, Berlin, 2005)). Second, these same agonists suppress activity in nociceptive neurons (Hohmann, A. G. et al., Life Sci. 56:2111-2118 (1995); Hohmann, A. G. et al., J. Neurophysiol. 81:575-583 (1999); Martin, W. J. et al., J. Neurosci. 16:6601-6611 (1996); Meng, I. D. et al., Nature 395:381-384 (1998)), in part through descending mechanisms (Hohmann, A. G. et al., J. Neurophysiol. 81:575-583 (1999); Meng, I. D. et al., Nature 395:381-384 (1998)). Third, CB₁ receptor antagonists alter activity of nociceptive neurons in the RVM (Meng, I. D. et al., Nature 395:381-384 (1998)) and enhance sensitivity to noxious stimuli (Calignano, A. et al., Nature 394:277-281 (1998)), suggesting that an intrinsic endocannabinoid tone may regulate descending analgesic pathways.

Anandamide, the naturally occurring amide of arachidonic acid with ethanolamine, meets all key criteria of an endogenous cannabinoid substance (Devane, W. A. et al. Science, 258: 1946-1949 (1992)): it is released upon demand by stimulated neurons (Di Marzo, V. et al., Nature, 372:686-691 (1994); Giuffrida, A. et al., Nat. Neurosci., 2:358-363 (1999)); it activates cannabinoid receptors with high affinity (Devane, W. A. et al. Science, 258: 1946-1949 (1992)) and it is rapidly eliminated through a two-step process consisting of carrier-mediated transport followed by intracellular hydrolysis (Di Marzo, V. et al., Nature, 372:686-691 (1994); Beltramo, M. et al., FEBS Lett., 403:263-267 (1997)). Anandamide hydrolysis is catalyzed by the enzyme fatty acid amide hydrolase (FAAH), a membrane-bound serine hydrolase (Cravatt, B. F. et al., Nature, 384:83-87 (1996); Patricelli, M. P. et al., Biochemistry, 38:9804-9812 (1999)) (WO 98/20119) that also cleaves other bioactive fatty ethanolamides, such as oleoylethanolamide (cis-9-octadecenamide)) (Rodríguez de Fonseca, F. et al. Nature, 414:209-212 (2001)) and palmitoylethanolamide (Calignano, A. et al., Nature, 394:277-281 (1998)). Mutant mice lacking the gene encoding for FAAH cannot metabolize anandamide (Cravatt, B. F. et al., Proc. Natl. Acad. Sci. U.S.A., 98:9371-9376 (2001)) and, though fertile and generally normal, show signs of enhanced anandamide activity at cannabinoid receptors, such as reduced pain sensation (Cravatt, B. F. et al., Proc. Natl. Acad. Sci. U.S.A., 98:9371-9376 (2001)). This suggests the possibility that drugs targeting FAAH may heighten the tonic actions of anandamide, while possibly avoiding the multiple, often unwanted effects produced by Δ⁹-THC and other direct-acting cannabinoid agonists ((Hall, 1998); Chaperon, 1999)).

Much attention has been directed toward the role of anandamide in pain. Methods of treating pain by administering anandamide and palmitylethanolamide are disclosed in U.S. Patent Application Publication No.: 20020173550. Methods of treating pain by administering inhibitors of FAAH are disclosed in U.S. Patent Application Publication Nos. 20040127518 and 20030134894. Methods of treating pain by administering inhibitors of anandamide transport are disclosed in U.S. Patent Application Publication No. 20030149082.

2-arachidonoylglycerol (2-AG), however, may also meet the defining criteria of an endocannabinoid. 2-AG is produced by neurons in an activity-dependent manner; engages CB₁ receptors with high affinity; and is rapidly eliminated through regulated transport and intracellular hydrolysis (Freund et al., Physiol. Rev. 83: 1017-1066 (2003)). In neurons, the hydrolysis of 2-AG is catalyzed by a serine hydrolase distinct from FAAH: monoacylglycerol lipase (MGL). MGL hydrolyzes 2-AG and other monoacylglycerols (Dinh, et al., Proc. Natl. Acad. Sci. USA 99(16):10819-24 (2002); Dinh, et al., Mol Pharmacol. 66(5):1260-4 (2004). The pharmacology of MGL, 2-AG, and other monoacylglycerols, and their relationship to each other, however, is not well understood.

The role of psychosocial stress (often referred to as ‘life events’) in triggering anxiety, PTSD, and the early episodes of depression has long been appreciated, and abnormalities in the ‘stress system’ of at least a subset of depressed individuals have been documented {Hossboer, F. Affect. Disord. 62, 77-91 (2001)]. The use of drugs that modulate the stress system represents therefore a new avenue for the treatment of anxiety disorders, PTSD and depression which are often resistant to classic anxiolytic and anti-depressant drugs.

Existing methods of treating pain are associated with side effects and often inadequate pain suppression as well as the development of tolerance. There is thus a considerable need for additional methods of treating pain and, therefore, considerable interest in understanding the biology of analgesia in order to develop new classes of analgesic agents to treat pain.

The present invention provides for these and other needs by disclosing the endocannabinoid involved stress-induced reduced responses, including, for instance, analgesia, and provides a new class of agents which operate upon the stress-induced responses, new methods for screening compounds to identify those compounds with the ability to modulate stress-induced responses, and new methods for treating pain or stress-related disorders by administering such compounds.

### BRIEF SUMMARY OF THE INVENTION

The present invention relates to the discovery that 2-arachidonoylglycerol (2-AG) is an endogenous neurotransmitter involved in the response of the central nervous system to stress and that stress-induced disorders (e.g., anxiety, post traumatic stress disorder, and depression) and analgesia can be treated by administration of an inhibitor of monacylglycerol lipase (MGL). Thus, in a first aspect, the invention provides for methods of modulating stress-induced homeostatic responses mediated by stress-induced release of 2-AG (e.g., stress-induced analgesia) by administering an inhibitor of MGL.

In a second aspect, the invention provides methods of treating pain or a stress-induced disorder (e.g., depression, anxiety, PTSD) by administering an inhibitor of MGL in a therapeutically effective amount. In one embodiment, the compound is a selective inhibitor of MGL (e.g., the compound has an IC₅₀ for inhibition of MGL that is one-third, one-fourth or one-tenth that of inhibiting the activity of a protein selected from FAAH or a pancreatic lipase). In another embodiment, the compound is selective for inhibition of MGL and has an IC₅₀ for inhibition of MGL that is one-third, one-fourth or one-tenth the concentration required to reduce anandamide transport by 50% or to modulate the activity of a PPAR subtype (e.g., γ, β, α, or δ) or a cannabinoid receptor (e.g., CB1 or CB2). In one embodiment, the MGL inhibitory compound is not otherwise an inhibitor of FAAH, of anandamide transport, or a cannabinoid CB1 and or CB2 receptor modulator (e.g., agonist or antagonist), or a PPAR receptor modulator (e.g., indirect or direct binding agonist or antagonist of the PPAR receptor subtype δ, γ, β, or α). In one embodiment, the compound is URB602:

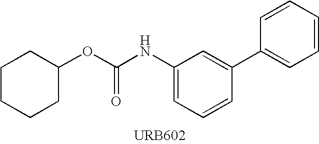

In another embodiment, the invention provides methods of treating pain or a stress-induced disorder by administering an inhibitor of MGL and one or more additional agents selected from analgesics, FAAH inhibitors, CB1 receptor agonists, anandamide transport inhibitors, opioids (e.g., morphine, hydromorphone, codeine, oxycodone, hydrocodone, methadone, levorphanol, fentanyl, meperidine, pentazocine, butorphanol tartrate, dezocine, and nalbuphine) and non-steroidal anti-inflammatory drugs and pain relievers (e.g., aspirin, acetaminophen), and COX-2 enzyme inhibitors (e.g., rofecoxib, celecoxib, and valdecoxib), and PPARα agonists (e.g., palmitylethanolamide).

In one further embodiment, the FAAH inhibitor is a compound of the formula:

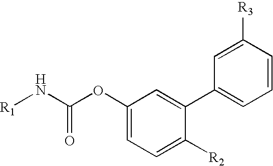

in which R¹ is independently selected from the group consisting of substituted or unsubstituted alkyl or substituted or unsubstituted cycloalkyl; R² and R³ are independently selected from the group consisting of H, C(O)NH₂, C(O)CH₃, CH₂OH, OH, NH₂. In a further embodiment, the compound is of the formula:

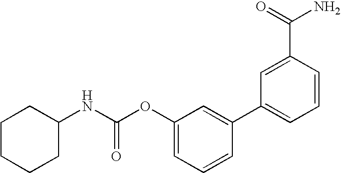

In another embodiment, the MGL inhibitor is MAFP:

### MAFP

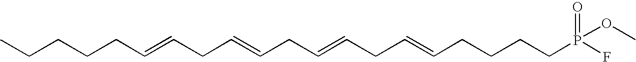

### methyl (5E,8E,11E,14E)-icosa-5,8,11,14-tetraenylphosphonofluoridate

In another embodiment, the invention provides a pharmaceutical composition comprising the MGL inhibitor and at least one other analgesic substance as provided above.

In a further aspect the invention provides, without being wed to theory, a method of treating pain or a stress-related disorder by administering a compound which is an MGL inhibitor or identified as being a MGL inhibitor.

In some embodiments of each of the above aspects involving pain, the pain is neuropathic pain, or pain initiated or caused by a primary lesion or dysfunction of the nervous system. The invention provides methods of treating all forms of neuropathic pain, including but not limited to, spontaneous pain, allodynia, and hyperalgesia.

In some embodiments, the pain can be a neuropathic pain selected from the group consisting of post trigeminal neuralgia, neuropathic low back pain, peripheral or polyneuropathic pain, complex regional pain syndrome, causalgia, and reflex sympathetic dystrophy, diabetic neuropathy, toxic neuropathy, and chronic neuropathy caused by chemotherapeutic agents. In other embodiments, the pain is renal and liver colic pain or fibromyalgia. In some neuropathic pain embodiments, the primary lesion or dysfunction of the nervous system is caused by a mechanical injury to a nerve of the subject. In a further embodiment, the mechanical injury is due to compression of a nerve, transection of nerve, causalgia, spinal cord injury, post surgical pain, phantom limb pain, or scar formation in the subject.

Metabolic and nutritional disorders can cause a primary lesion or dysfunction of the nervous system responsible for neuropathic pain. In some embodiments, the primary lesion or dysfunction of the nervous system is a diabetic neuropathy, pellagric neuropathy, alcoholic neuropathy, Beriberi neuropathy, or burning feet syndrome. In still other embodiments, the subject has a neurological disease (e.g., multiple sclerosis, trigeminal neuralgia, Guillain-Barre syndrome, Fabry's disease, or Tangier disease) which caused the primary lesion or dysfunction of the nervous system. In particular embodiments, the neuropathic pain is a complex regional pain syndrome, sciatica, or diabetic neuropathy.

In other embodiments, the pain is a pain caused by inflammation of a tissue. Inflammatory pain develops in response to tissue damage occurring from the noxious stimuli. In response to the tissue injury, cytokines and other mediators are released which strengthen nociception. As a result primary hyperalgesia (increased sensitivity to pain) occurring in the area of injury and a secondary hyperalgesia occurring in the tissue surrounding the injury ensue. The hyperalgesia subsides with the inflammation as the tissue is healed. In some further embodiments, the inflammation is associated with pulmonary edema, kidney stones, minor injuries, wound healing, skin wound healing, vaginitis, candidiasis, lumbar spondylanhrosis, lumbar spondylarthrosis, vascular diseases, migraine headaches, sinus headaches, tension headaches, dental pain, periarteritis nodosa, thyroiditis, aplastic anemia, Hodgkin's disease, sclerodoma, rheumatic fever, type I diabetes, type II diabetes, myasthenia gravis, multiple sclerosis, sarcoidosis, nephrotic syndrome, Behcet's syndrome, polymyositis, gingivitis, hypersensitivity, swelling occurring after injury, or myocardial ischemia.

In yet other embodiments, of the above aspects involving stress-related disorders, the stress-related disorder can be an anxiety disorder, post-traumatic stress disorder (PTSD), or depression and the MGL inhibitor can be administered alone or in combination with one or more agents targeting the condition or endogenous cannabinoid system. Such agents include, but are not limited to, a FAAH inhibitor, a PPARα agonist, a CB1 or CB2 cannabinoid receptor agonist, an anandamide transport inhibitor, analgesics, opioids, anxiolytics, and antidepressants. The agents may be formulated together or separately.

Accordingly, in a second aspect, the invention provides for a method of screening a compound for use in modulating a stress-induced homeostatic response mediated by 2-AG, in treating pain or in treating a stress-induced disorder (e.g., anxiety, depression, post-traumatic stress disorder (PTSD)) by contacting the compound with MGL and determining the ability of the compound to inhibit the MGL enzyme. In one embodiment, the method measures the catalytic activity of MGL in hydrolyzing monoacylglycerol as a substrate. In another embodiment, the substrate for MGL is 2-AG. In one embodiment, the MGL substrate is labeled and the method detects the amount of a labeled product formed from the hydrolysis of the substrate. In another embodiment, the MGL is a mammalian MGL (e.g., MGL from rodent, rat, mouse, or human nervous system tissue). In other embodiments, a compound identified to inhibit the catalytic activity of MGL is further tested in an in vivo model in a mammal for modulation of stress-analgesia to confirm the ability of the compound to modulate pain or stress-induced analgesia in vivo.

In a fourth aspect, the invention provides novel compounds or inhibitors of MGL of based upon URB 602 as discussed infra. In further aspects the invention provides pharmaceutical compositions comprising these compounds and for methods of treating the above conditions and diseases using the novel compounds and compositions.

In a fifth aspect, the invention provides methods of neuroprotection in response to stroke or ischemia or a brain inflammatory diseases by administering a MGL inhibitor to a subject in need of such neuroprotection. The MGL inhibitor can be administered alone or in combination with one or more agents targeting the condition or endogenous cannabinoid system. Such additional agents include, but are not limited to, a FAAH inhibitor, a PPARα agonist, a CB1 or CB2 cannabinoid receptor agonist, an anandamide transport inhibitor, analgesics, opioids, anxiolytics, and antidepressants. The agents may be formulated together or separately.

In some other aspects, the invention provides methods of 1) treating pain or stress-induced analgesia in a mammalian subject in need thereof; 2) methods for treating a stress-induced disorder or condition in a mammalian subject in need thereof; 3) methods for enhancing or potentiating stress-induced analgesia in a mammalian subject in need thereof, and 4) methods of producing analgesia in a patient in need thereof, wherein the patient is tolerant to morphine which use compounds according to the invention.

### BRIEF DESCRIPTION OF THE DRAWINGS

FIG. 1. CB₁ receptors mediate non-opioid stress analgesia. a, The CB₁ antagonist rimonabant (Ri; 5 mg-kg⁻¹, i.p.), but not the opioid antagonist naltrexone (N; 14 mg-kg⁻¹, i.p.) or the CB₂ antagonist SR144528 (S; 5 mg-kg⁻¹, i.p.), prevents stress-induced analgesia; V, vehicle. Inset: effect of rimonabant for the observation interval (F_{3,29}=5.99). Dotted line denotes baseline nociceptive threshold. b, Rimonabant (5 mg-kg⁻¹, i.p.) does not alter tail-flick latencies in absence of stressor. c, Non-opioid stress analgesia is attenuated in rats tolerant to the cannabinoid agonist WIN55, 212-2 (10 mg-kg⁻¹, i.p. for 14 days) (F_{1,19}=16.74, P<0.0007). Inset: repeated daily injections of WIN55, 212-2 decrease the drug's antinociceptive effects prior to foot shock (F_{2,38}=35.11, P<0.0002). d, Non-opioid stress analgesia is unaffected by morphine tolerance (10 mg-kg⁻¹, s.c. daily for 7 days). Inset: repeated daily injections of morphine decrease the drug's antinociceptive effects prior to foot shock (F_{1,14}=121.63, P<0.0002). e,f, Rimonabant (2 nmol; Ri 7 min before foot shock) suppresses stress analgesia (F_{10,170}=20.01, P<0.0002) (e) in the dorsolateral but not (f) ventrolateral PAG. Inset: effects of rimonabant for the observation interval (F_{1,17}=25.63, P<0.0002). a-f, Mean±SEM. *P<0.05, **P<0.01, ***P<0.001 versus other groups (ANOVA, Fisher's PLSD test). g,h, Coronal reconstructions of injection sites for e and f, respectively: vehicle, open circles; rimonabant, closed circles.

FIG. 2. Stress stimulates formation of 2-AG and anandamide in dorsal midbrain. a,b, Representative LC/MS tracings for selected ions characteristic of (a) endogenous 2-AG (top, mass-to-charge ratio, m/z=401.3), an adduct with Na⁺, [M+Na⁺], and synthetic [²H₈]-2-AG (bottom, m/z=409.3), which was added to the samples as an internal standard. The first peak corresponds to 1(3)-AG, a 2-AG isomer produced during sample preparation (Stella, N. et al., Nature 388:773-778 (1997)), which was summed with 2-AG for quantification purposes; and (b) endogenous anandamide (top, m/z=370.3, [M+Na⁺]) and synthetic [²H₄]-2-anandamide (bottom, m/z=374.3) c,d, Non-shock (open squares) or post-shock (closed circles) levels of 2-AG and anandamide in samples of (c) dorsal midbrain containing intact PAG, or (d) occipital cortex. *P<0.05, **P<0.01 versus non-shocked control.

FIG. 3. URB602 is a selective MGL inhibitor. a, Structures of O-biphenyl substituted FAAH inhibitors (1, URB597; 2, URB524) and the N-biphenyl substituted MGL inhibitor URB602 (3). b, URB602 (circles) inhibits rat brain MGL activity in vitro, whereas URB597 (squares) and URB524 (triangles) have no effect. c, URB602 does not affect rat brain FAAH activity, which is suppressed by URB597 and URB524. d,e, URB602 (100 μM) (d) increases 2-AG but not (e) anandamide levels in organotypic brain slice cultures. Effects of the calcium ionophore ionomycin (2 μM) and URB597 (1 μM) are shown. **P<0.01, ***P<0.001 versus control, t-test (n=4).

FIG. 4. The MGL inhibitor URB602 enhances non-opioid stress analgesia. a,b, URB602 (602; 0.1 nmol) potentiates stress analgesia in (a) dorsolateral and (b) ventrolateral PAG; rimonabant (Ri) blocks this effect at a dose (0.2 nmol) that does not reverse stress analgesia. Insets: effects for the observation interval (a, F_{3,26}=7.39, P<0.002; b, F_{3,25}=9.15, P<0.0004); Mean±SEM. *P<0.05, **P<0.01, ***P<0.001 versus all groups; ^{x}P<0.05, ^{xx}P<0.01 versus vehicle; ^{#}P<0.05 versus URB602 plus rimonabant; (ANOVA, Fisher's PLSD test). Dotted line denotes baseline nociceptive threshold. c,d,f, Coronal reconstructions of injection sites for a,b,e, respectively; Vehicle, open circles; URB602, closed squares; rimonabant, closed circles; URB602 plus rimonabant, open squares. e, URB602 (0.1 nmol in ventrolateral PAG) does not induce antinociception in non-shocked rats. g, URB602 (0.1 nmol) microinjection in the ventrolateral PAG increases 2-AG but not anandamide. Endocannabinoids were measured 25 min after shock. h, URB602 (0.1 nmol) in the PAG did not alter endocannabinoid levels in occipital cortex.

FIG. 5. Inhibition of anandamide hydrolysis and transport enhances non-opioid stress analgesia. The FAAH inhibitor URB597 (597), administered (a) systemically (0.3 mg-kg⁻¹ i.p.) or (b) into the dorsolateral PAG (3 nmol), and the (d) anandamide transport inhibitor VDM11 (10 mg-kg⁻¹ i.p.) increase stress analgesia. Rimonabant (Ri) blocked these effects following i.p. (a, 1 mg-kg⁻¹; d, 2 mg-kg⁻¹) or (b) intra-PAG (0.1 nmol) administration. c, Coronal reconstructions of injection sites in b. Insets: effects of (a) systemic URB597 (F_{3,28}=11.56, P<0.0002), (b) microinjected URB597 (F_{3,24}=33.69, P<0.0002), and (d) systemic VDM11 (F_{3,26}=21.76, P<0.0002) for the observation interval; Mean±SEM. *P<0.05, **P<0.01, ***<P<0.001, versus all groups; ^{x}P<0.05, ^{xx}P<0.01 versus vehicle; ^{#}P<0.05 versus rimonabant conditions (ANOVA, Fisher's PLSD post hoc test). Dotted line denotes baseline nociceptive threshold.

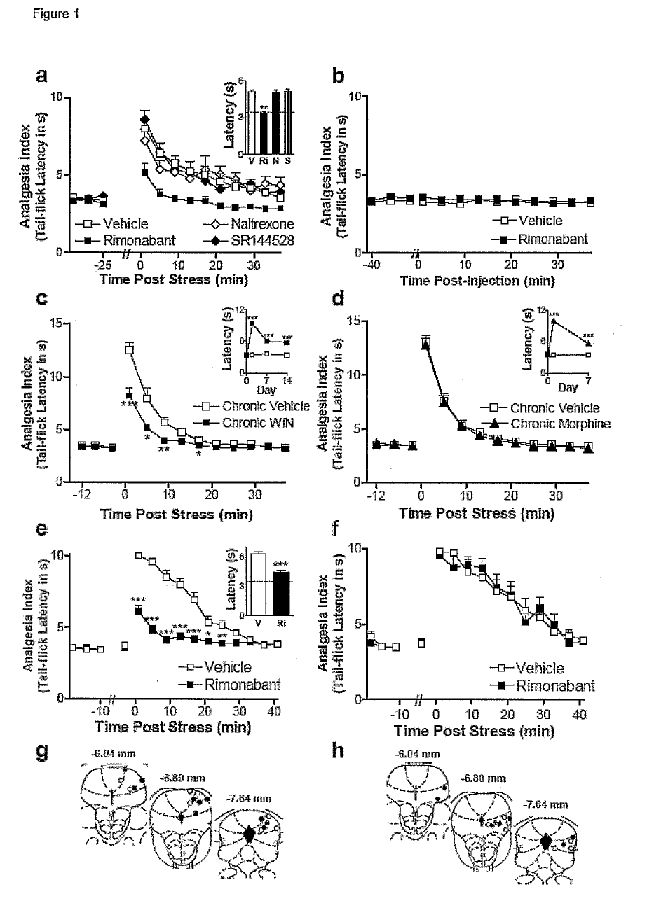

Supplementary FIG. 1. Systemic administration of the CB₁ antagonist AM251 (5 mg-kg⁻¹ i.p.) suppresses non-opioid stress analgesia. Inset: effect of AM251 for the observation interval (F_{1,22}=18.32, P<0.0004) Mean±SEM. *P<0.05, **P<0.01, ***P<0.001 versus control (ANOVA, Fisher's PLSD post hoc test).

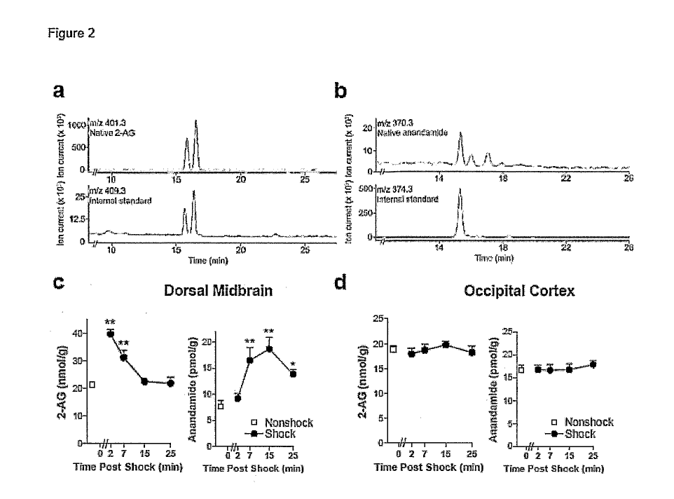

Supplementary FIG. 2. Administration of rimonabant (2-10 nmol) into the lateral ventricle does not affect opioid-independent stress analgesia. Mean±SEM.

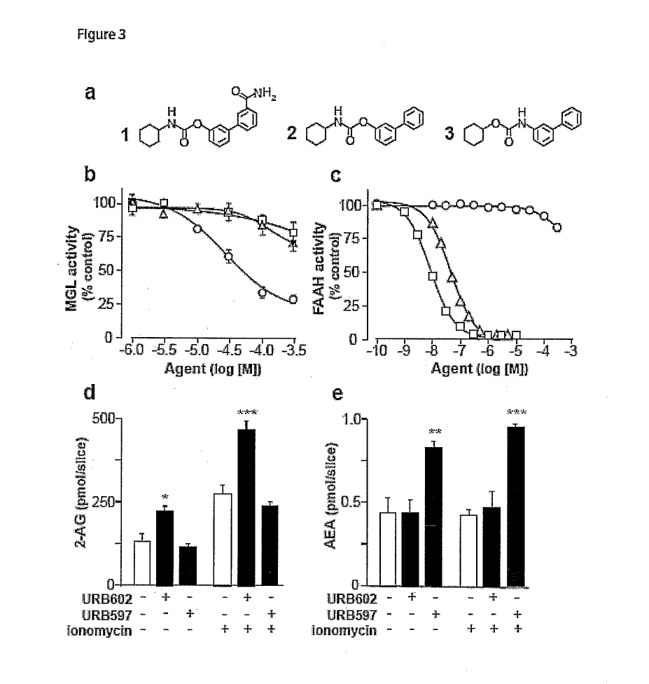

Supplementary FIG. 3.a, Microinjection of the non-selective MGL inhibitor methylarachidonylfluorophosphate (MAFP) (2.6, 26 nmol) in the dorsolateral PAG enhances opioid-independent stress analgesia. Co-injection of rimonabant (Ri; 0.2 nmol) blocks this effect. Drugs were administered 35 min before foot shock. Inset: effect for the observation interval (F_{4,33}=21.92, P<0.0002). Mean±SEM. *P<0.05, **P<0.01, ***P<0.001 versus control conditions; ^{x}P<0.05 versus vehicle; ⁺P<0.05, ⁺⁺⁺P<0.001 versus MAFP plus rimonabant (ANOVA, Fisher's PLSD post hoc test). b, Coronal reconstructions of injection sites in a: Vehicle, open circles; MAFP (2.6 nmol), closed squares; MAFP (26 nmol), closed triangles; Rimonabant, closed circles; Rimonabant plus MAFP, open triangles.

### DETAILED DESCRIPTION OF THE INVENTION

This invention relates to our finding that MGL and the 2-AG stress response system as a novel therapeutic target for treating stress-related and pain-related disorders. Acute stress suppresses pain by activating brain pathways that engage both opioid and non-opioid mechanisms. Injection of CB1 cannabinoid receptor antagonists into the periaqueductal gray matter (PAG) of the midbrain can prevent non-opioid stress-induced analgesia. In this region, we have now further found that stress elicits the rapid formation of two endocannabinoids, 2-arachidonoylglycerol (2-AG) and anandamide. In addition, we have found that inhibitors of the 2-AG-deactivating enzyme monoacylglycerol lipase (MGL) can selectively increase 2-AG levels and, when injected into the PAG, magnify stress-induced analgesia in a CB1-dependent manner. The invention also provides novel MGL inhibitors which allow the effects of MGL inhibition on anti-nociception and stress-induced analgesia. We also show that fatty-acid amide hydrolase inhibitors, which selectively elevate anandamide levels, can also magnify stress-induced analgesia. These results indicate that release of both 2-AG and anandamide in the PAG mediates opioid-independent stress-induced analgesia, and thus identify MGL as a novel drug target in therapy and screening.

Accordingly, the invention provides new methods for screening for or identifying compounds having anxiolytic, anti-depressant, or antinociceptive activity by administering a MGL lipase inhibitor; and new methods of modulating stress-induced responses or conditions by administering a MGL lipase inhibitor, and new MGL inhibitory compounds. The invention also provides related pharmaceutical compositions for use in treating pain and providing anti-nociception as well as in treating stress-induced disorders, including but not limited to, anxiety, depression, behavioral addictions—including chemical or drug dependencies, and PTSD.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

Unless defined otherwise, all technical and scientific terms used herein have the meaning commonly understood by a person skilled in the art to which this invention belongs. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991). As used herein, the following terms have the meanings ascribed to them unless specified otherwise.

It is noted here that as used in this specification and the appended claims, the singular forms “a,” “an,” and “the” include plural reference unless the context clearly dictates otherwise.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. The term “pharmaceutical composition” indicates a composition suitable for pharmaceutical use in a subject, including an animal or human. A pharmaceutical composition generally comprises an effective amount of an active agent and a pharmaceutically acceptable carrier.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in REMINGTON'S PHARMACEUTICAL SCIENCES (Mack Publishing Co., Easton, 19th ed. 1995). Preferred pharmaceutical carriers depend upon the intended mode of administration of the active agent. Typical modes of administration are described below.

The term “effective amount” means a dosage sufficient to produce a desired result on health, including, but not limited to, disease states. The desired result may comprise a subjective or objective improvement in the recipient of the dosage. A subjective improvement may be, for instance with respect to pain, decreased sensation of pain (e.g., noninflammatory pain, neuropathic pain). An objective improvement may be, for instance, an increased ability to move or use (e.g., place weight upon) an affected limb or a longer period of uninterrupted sleep, or a behavioral response indicating an increased tolerance of a painful stimuli.

A “prophylactic treatment” is a treatment administered to a subject who does not have the subject condition (e.g., pain), wherein the treatment is administered for the purpose of decreasing the risk of developing the condition or to counter the severity of the condition (e.g., anxiety; depression; pain, including but not limited to, acute pain, chronic pain, inflammatory pain, non-inflammatory pain, neuropathic pain and pain expected to result from the expected or likely occurrence of a painful event (e.g., surgery)) if one were to develop.

A “therapeutic treatment” is a treatment administered to a subject who has the condition (e.g., pain, and/or exhibits signs or symptoms of pain including but not limited to, acute pain, chronic pain, cancer pain, inflammatory pain, non-inflammatory pain, neuropathic pain, wherein treatment is administered for the purpose of diminishing or eliminating those signs or symptoms) to be treated.

A “therapeutically effective amount” is an amount of an agent sufficient to reduce the signs and/or symptoms of the disease or condition or to prevent, oppose, or reduce their progression.

The term “modulate” means to induce any change including increasing and decreasing. A modulator of a receptor includes both agonists and antagonists of the receptor.

The term “treating” means combating, reducing, shortening, alleviating or eliminating a condition of the subject (e.g., pain, anxiety, or depression).

Pain, particularly severe pain, can be a stressor. Thus, in one aspect the invention is drawn to methods of treating chronic pain conditions, including neuropathic pain, and chronic or intermittent pain associated with chronic health conditions as such conditions are often substantial stressors. “Neuropathic pain” is pain caused by a primary lesion or dysfunction of the nervous system. Such pain is chronic and involves a maintained abnormal state of increased pain sensation, in which a reduction of pain threshold and the like are continued, due to persistent functional abnormalities ensuing from an injury or degeneration of a nerve, plexus or perineural soft tissue. Such injury or degeneration may be caused by wound, compression, infection, cancer, ischemia, or a metabolic or nutritional disorder such as diabetes mellitus. Neuropathic pain includes, but is not limited to, neuropathic allodynia wherein a pain sensation is induced by mechanical, thermal or another stimulus that does not normally provoke pain, neuropathic hyperalgesia wherein an excessive pain occurs in response to a stimulus that is normally less painful than experienced. Examples of neuropathic pain include diabetic polyneuropathy, entrapment neuropathy, phantom pain, thalamic pain after stroke, post-herpetic neuralgia, atypical facial neuralgia pain after tooth extraction and the like, spinal cord injury, trigeminal neuralgia and cancer pain resistant to narcotic analgesics such as morphine. The neuropathic pain includes the pain caused by either central or peripheral nerve damage. And it includes the pain caused by either mononeuropathy or polyneuropathy (e.g., familial amyloid polyneuropathy). As compared to inflammatory pain, neuropathic pain is relatively resistant to therapy with nonsteroidal anti-inflammatory agents and opioid substances (e.g, morphine).

Neuropathic pain may be bilateral in mirror image sites, or may be distributed approximately according to the innervation of the injured nerve, it may persist for months or years, and be experienced as a burning, stabbing, shooting, throbbing, piercing electric shock, or other unpleasant sensation.

Anxiety is a state of fearfulness which is unprovoked by an environmental threat or highly disproportionate to an environmental threat. Anxiety may be acute and short term lasting hours to days; or chronic and lasting from many days to weeks or longer. The term includes clinical anxiety which refers to any form of anxiety for which treatment is necessary or indicated in order to alleviate it. Such clinical anxiety may be persistent or recurrent and typically severe and includes anxiety disorders. Anxiety disorders include, but are not limited to, any of the anxiety disorders as provided in the Diagnostic and Statistical Manual of Mental Disorders, Fourth Edition. (Copyright 1994 American Psychiatric Association) which is hereby incorporated by reference. Such disorders include, but are not limited to, panic disorder, agoraphobia, generalized anxiety disorder, specific phobia, social phobia, obsessive-compulsive disorder, acute stress disorder, and post-traumatic stress disorder; and adjustment disorders with anxious features, anxiety disorders due to general medical conditions, substance-induced anxiety disorders, and the residual category of anxiety disorder not otherwise specified.

Depressive disorders and conditions include, but are not limited to, any of the depressive disorders and conditions as provided in the Diagnostic and Statistical Manual of Mental Disorders, Fourth Edition (Copyright 1994 American Psychiatric Association). These disorders include major depressive disorder (unipolar depression), dysthymic disorder (chronic, mild depression), and bipolar disorder (manic-depression). Clinical depression refers to any form of depression that requires some form of treatment in order to alleviate it. Such clinical depression may persist for months and last for most of every day and seriously impairs the quality of life.

A “major depressive episode” is defined as at least two weeks of depressed mood or loss of interest, which may be accompanied by other symptoms of depression. The symptoms must persist for most of the day (i.e. for at least two thirds of the patients' waking hours), nearly every day (i.e. for at least ten out of fourteen days) for at least two consecutive weeks. A “depressed mood” is often described by the patient as feeling sad, hopeless, helpless or worthless. The patient may also appear sad to an observer, for example, through facial expression, posture, voice and tearfulness. In children and adolescents, the mood may be irritable. A “loss of interest” is often described by the patient as feeling less interested in hobbies or not feeling any enjoyment in activities that were previously considered to be pleasurable.

A major depressive episode may be accompanied by other symptoms of depression including significant weight loss when not dieting or weight gain (e.g. a change of more than 5% body weight in one month), or decrease or increase in appetite; insomnia or hypersomnia; psychomotor agitation or retardation; fatigue or loss of energy; feelings of worthlessness or excessive or inappropriate guilt; diminished ability to think or concentrate; or indecisiveness; and recurrent thoughts of death, recurrent suicidal ideation with or without a specific plan, or a suicide attempt.

The term “post-traumatic stress disorder” is known to one of ordinary skill in the art who can readily diagnosis this condition. The condition generally results from experiencing or witnessing an extremely upsetting event capable of or causing severe physical or emotional harm to the subject or another, particularly, a loved one. The response typically involve great fear, helplessness, horror, or disorganized or agitated behavior. The event may be involve ‘flashbacks’ and be associated with avoidance of reminders of the event. The general responsiveness of the subject to his environment may be numbed and their may be persistent symptoms of increased arousal to provoking events. The condition is often very persistent, lasting at least weeks to months, and can result in substantial emotional distress or social and occupational impairment.

The subject species to which the treatments can be given according to the invention are mammals, and include, but are not limited to, humans, primates, rodents, rats, mice, rabbits, horses, dogs and cats. In preferred embodiments of each aspect, the subject is human.

Peroxisome proliferator activated receptors (PPAR) are a family of transcription factors and have been principally studied with respect to lipid homeostasis. Three PPAR subtypes have been identified: α, β, (also described as δ), and γ. All three subtypes have domain structure common with other members of the nuclear receptor family. DNA-binding domains are highly conserved among PPAR subtypes, but ligand binding domains are less well conserved. (Willson, et al., J. Med. Chem. 43:527 (2000). PPARs bind to RXR transcription factors to form heterodimers that bind to DNA sequences containing AGGTCAnAGGTCA. It has been shown that ligand binding to PPAR can induce gene expression. PPARα has been reported to inhibit inflammatory edema and inflammatory pain (see Taylor et al. Inflammation 26(3): 121 (2002) and Sheu et al. J. Invest. Dermatol. 118:94 (2002)). Suitable PPARα agonists, CB₁ receptor agonists, and FAAH inhibitors, and anandamide transport inhibitors for use according to the present invention are disclosed in U.S. Provisional Patent Application No. 60/565,196, filed Apr. 23, 2004 and assigned to the same assignee as the present application, and incorporated by reference herein in its entirety and particularly with respect to the PPARα agonist (e.g., PPARα activator, partial agonist, full agonist), CB₁ receptor agonist, FAAH inhibitor, and anandamide transport inhibitor subject matter disclosed therein.

### Compounds for Use According to the Invention

Compounds for use according to the present invention (e.g., MGL inhibitors, FAAH inhibitors, anandamide transport inhibitors, CB1 receptor agonists, PPARα receptor agonists) may contain one or more asymmetric centers and can thus occur as racemates and racemic mixtures, single enantiomers, diastereomeric mixtures and individual diastereomers. Some of the compounds described herein contain olefinic double bonds, and unless specified otherwise, are can include both E and Z geometric isomers. The present invention is meant to comprehend all such pharmacologically active forms of the inventive compounds, including, but not limited to, their polymorphs.

Some of the compounds described herein may exist with different points of attachment of hydrogen, referred to as tautomers. Such an example may be a ketone and its enol form known as keto-enol tautomers. The pharmacologically active individual tautomers as well as their tautomer are encompassed by the inventive formulas.

Compounds of the invention include the pharmacologically active diastereoisomers of pairs of enantiomers and their racemates. Diastereomers for example, can be obtained by fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent or stereospecific synthesis using optically pure starting materials or reagents of known configuration.

The compounds for use according to the present invention may have unnatural ratios of atomic isotopes at one or more of their atoms. For example, the compounds may be radiolabeled with isotopes, such as tritium or carbon-14. All isotopic variations of the compounds of the present invention, whether radioactive or not, are within the scope of the present invention. The compounds include their pharmaceutically acceptable solvates, salts, polymorphs, tautomers, racemates and/or individual enantiomers.

The compounds may be isolated in the form of their pharmaceutically acceptable acid addition salts, such as the salts derived from using inorganic and organic acids. Such acids may include hydrochloric, nitric, sulfuric, phosphoric, formic, acetic, trifluoroacetic, propionic, maleic, succinic, malonic and the like. In addition, certain compounds containing an acidic function can be in the form of their inorganic salt in which the counterion can be selected from sodium, potassium, lithium, calcium, magnesium and the like, as well as from organic bases. The term “pharmaceutically acceptable salts” refers to salts prepared from pharmaceutically acceptable non-toxic bases or acids including inorganic bases or acids and organic bases or acids.

The invention also encompasses prodrugs of MGL inhibitors and FAAH inhibitors which on administration undergo chemical conversion by metabolic processes before becoming active pharmacological substances. In general, such prodrugs will be derivatives of the present compounds that are readily convertible in vivo into a functional compound of the invention. Conventional procedures for the selection and preparation of suitable prodrug derivatives are described, for example, in “Design of Prodrugs”, ed. H. Bundgaard, Elsevier, 1985. The invention also encompasses active metabolites of the present compounds.

Where substituent groups are specified by their conventional chemical formulae, written from left to right, they equally encompass the chemically identical substituents which would result from writing the structure from right to left, e.g., —CH₂O— is intended to also recite —OCH₂—. As used herein, the term “heteroatom” is meant to include oxygen (O), nitrogen (N), sulfur (S) and silicon (Si).

### MGL Inhibitors

A variety of lipase inhibitors may be used as an MGL inhibitor according to the invention. As noted above, the compounds include their pharmaceutically acceptable solvates, salts, polymorphs, tautomers, racemates and/or individual enantiomers.

### MGL Inhibitors Related to URB602

In one embodiment, the inhibitor may be a compound of the formula:

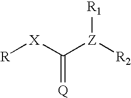

in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and

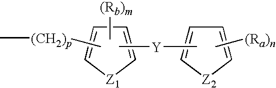

wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring.

In addition, R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached.

URB602 is an exemplary example of such a lipase inhibitor for use according to the invention.

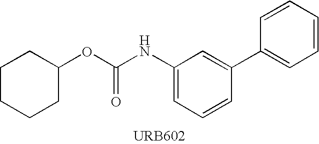

### Oxazinone Lipase Inhibitors

Oxazinone lipase inhibitors which may be used according to the invention are disclosed in United States Patent Application Publication Nos. 20030191123; 20030176429; 20030013707; 20030027821 and in U.S. Pat. No. 6,656,934 which are incorporated herein by reference in their entireties and particularly with respect to the lipase inhibitors disclosed therein. Thienoxazin-4-ones as lipase inhibitors are also disclosed in International PCT Patent Application Publication No. WO 2003020282 which is also incorporated herein by reference in its entirety with respect to such lipase inhibitor subject matter. These reference concerns lipase inhibitors of the following formulae:

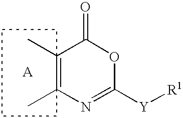

in which A is an optionally substituted 6-membered aromatic or heteroaromatic ring; Y is O, S, or NR²; and R¹ is a branched or unbranched alkyl (optionally interrupted by one or more oxygen atoms), alkenyl, alkynyl, cycloalkyl, cycloalkenyl, aryl, arylalkyl, reduced arylalkyl, arylalkenyl, heteroaryl, heteroarylalkyl, heteroarylalkenyl, reduced aryl, reduced heteroaryl, reduced heteroarylalkyl or a substituted derivative of any of the foregoing groups, wherein the substituents are one or more independently of halogen, alkyl, halosubstituted alkyl, aryl, arylalkyl, heteroaryl, reduced heteroaryl, reduced heteroarylalkyl, arylalkoxy, cyano, nitro, —C(O)R⁴, —CO₂R⁵, —SOR⁴, —SO²R⁴, —NR⁶R⁷, —OR⁶, —SR⁶, —C(O)CX¹X²NR⁶R⁷, —C(O)N(OH)R⁶, —C(O)NR⁵R⁴0, —NR6C(O)R⁴, —CR⁶(NH₂)CO₂R⁶, —NHCX¹X.²CO₂R⁶, —N(OH)C(O)NR⁶R⁷, —N(OH)C(O)R⁴, —NHC(O)NR⁶R⁷, —C(O)NHNR⁶R⁷, —C(O)N(OR⁵)R⁶, or a lipid or steroid (natural or synthetic) with the proviso that any hetero atom substituent in R¹ must be separated from the exocyclic sulphur atom by at least two carbon atoms (preferably saturated); and where: R⁴ is hydrogen, alkyl, alkenyl, alkynyl, cycloalkyl, cycloalkenyl, aryl, arylalkyl, heteroaryl, heteroarylalkyl, reduced heteroaryl, reduced heteroarylalkyl, —OR⁶, —NHCX¹X²CO₂R⁶ or —NR⁶R⁷; R⁵ is hydrogen, alkyl, alkenyl, alkyl, cycloalkyl, cycloalkenyl, aryl, arylalkyl heteroaryl, heteroarylalkyl, reduced heteroaryl or reduced heteroarylalkyl; and R⁶ and R⁷ are independently hydrogen, alkyl, alkenyl, alkynyl, cycloalkyl, cycloalkenyl, aryl, arylalkyl, heteroaryl, reduced heteroaryl, reduced heteroarylalkyl or —(CH₂)ₙ(OR⁵)ₘ wherein n is 1 to 12, preferably 2 to 10, wherein m is 1-3 and R⁵ is most preferably C₂-C₁₀alkyl; and X¹ and X² are independently hydrogen, alkyl, alkenyl, alkynyl, cycloalkyl, cycloalkenyl, aryl, heteroaryl, arylalkyl, heteroarylalkyl, reduced heteroaryl or reduced heteroarylalkyl. R² is H or a group as defined for R¹. See U.S. Patent Application Publication No. 20030176429 for these and other such compounds for use according to the invention.

Also included are compounds of the formula:

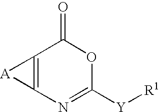

in which A can be a substituted or unsubstituted thienyl moiety; Y can be O, S, or NR²; and R¹ can be substituted or unsubstituted alkyl optionally interrupted by one or more O atoms, alkenyl, cycloalkenyl, alkynyl, cycloalkyl, aryl, heteroaryl, arylalkyl, heteroarylalkyl, reduced arylalkyl or heteroarylalkyl, arylalkenyl, heteroarylalkenyl, or reduced aryl or heteroaryl; R² can be H or a group as defined for R¹.

In one embodiment, the compound is 2-phenylamino-4H-thieno[2,3-d][1,3]oxazin-4-one:

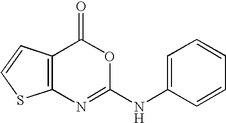

2-thio-4H-3,1-benzoxazin-4-ones lipase inhibitors which may be used according to the invention are disclosed in U.S. Patent Application Publication No. 2003176429 which is incorporated herein by reference. Such compounds are of the formula:

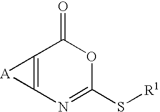

In this embodiment, a suitable MGL inhibitor for use according to the invention has an A member in which the A atoms form a substituted or unsubstituted 6-membered heteroaromatic ring; and R¹ can be a substituted or unsubstituted O-interrupted alkyl, alkenyl, alkynyl, cycloalkyl, cycloalkenyl, aryl, aralkyl, or heteroaryl member. In one embodiment, the inhibitor for use according to the invention is the 2-octylthio-6-methyl-4H-3,1-benzoxazin-4-one.

Lipase inhibitors which may be used according to the invention can also be compounds as disclosed in U.S. Patent Application Publication No. 20030195199 to Witter et al., which is incorporated by reference herein in its entirety. This reference teaches suitable lipase inhibitors of the formulae:

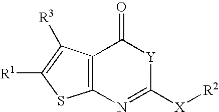

in which X can be O, S, CH₂, or NR⁵; Y can be O or S; and R¹ can be H, substituted or unsubstituted alkyl or aryl, CO₂R⁴, CONR⁴R⁵, CR⁶R¹⁰OR⁴, CR⁶R¹⁰OCOR⁴, CR⁶R¹⁰OCONHR⁷, CONR⁸R⁹, NR⁵CONHR⁵, or CH₂R⁴; wherein R² can be substituted or unsubstituted alkyl, aryl, alkylaryl, heteroarylalkyl, or cycloalkyl; R³ can be H or substituted or unsubstituted alkyl, cycloalkyl; R⁴ can be H, substituted or unsubstituted alkyl, aryl, CH₂-aryl, arylalkyl, heteroarylalkyl, or cycloalkyl; R⁵ can be H, substituted or unsubstituted alkyl, arylakyl or hetero)rylalkyl, or cycloalkyl; R⁶ and R¹⁰ can be independently H or substituted or unsubstituted alkyl or cycloalkyl; or R⁶ and R¹⁰ taken together can optionally form a ring; R⁷ can be H or substituted or unsubstituted alkyl or cycloalkyl; R⁸ and R⁹ can be independently H, substituted or unsubstituted alkyl, alkoxy, or alkylaryl; or NR⁸R⁹ taken together can form a substituted piperazine ring, a piperidine ring, or a dihydro-1H-isoquinoline ring.

In one embodiment, the lipase inhibitor may be of the formula:

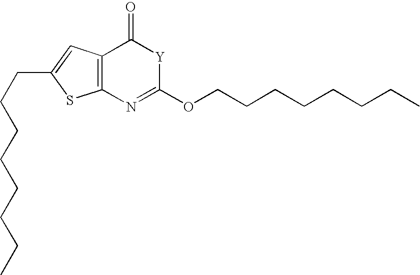

### Carbamates, Carbazates, Carbamoyloximes, Carbamoyltriazole, Carbamoylisoxazoline Lipase Inhibitors.

Additional lipase inhibitors which may be used according to the present invention include compounds of the general formula:

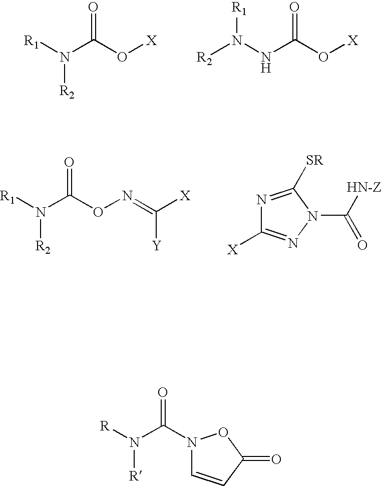

Such compounds are disclosed in U.S. Patent Application Publication Nos. 20030166690 and 20030166644, and PCT Patent Application Publication Nos. WO2003051842; WO2003051841 which are incorporated herein by reference in their entireties.

These compound include compounds of the formulae as taught in U.S. Patent Application Publication No. 20030166690:

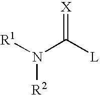

wherein R¹ can be H or substituted or unsubstituted alkyl or cycloalkyl or alkenyl; R² can be substituted or unsubstituted alkyl or cycloalkyl, alkenyl, aryl or heteroaryl, or heterocyclyl; or NR¹R² can be heterocyclyl; X can be O or S; and L can be a hydrolyzable group. For instance, R¹ can be selected from hydrogen, C₁₋₆-alkyl, C₂₋₆-alkenyl and C₃₋₁₀-cycloalkyl, each of which can be optionally substituted with one or more substituents independently selected from hydroxy, sulfanyl, oxo, halogen, amino, cyano and nitro; and R² can be selected from C₁₋₆-alkyl, C₂₋₆-alkenyl, aryl, heteroaryl, C₃₋₈-heterocyclyl and C₃₋₁₀-cycloalkyl, each of which can be optionally substituted with one or more substituents independently selected from hydroxy, sulfanyl, sulfo, oxo, halogen, amino, cyano, nitro, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, amino, C₁₋₆-alkyl, C₂₋₆-alkenyl, aryl, heteroaryl, C₃₋₈heterocyclyl and C₃₋₁₀-cycloalkyl can be optionally substituted with one or more substituents independently selected from hydroxy, sulfanyl, sulfo, oxo, halogen, amino, cyano, nitro, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, amino, C₁₋₆-alkyl, C₂₋₆-alkenyl, aryl, heteroaryl, C₃₋₈-heterocyclyl and C₃₋₁₀-cycloalkyl can be optionally substituted with one or more substituents independently selected from hydroxy, sulfanyl, sulfo, oxo, halogen, amino, cyano, nitro, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, amino, C₁₋₆-alkyl, C₂₋₆-alkenyl, aryl, heteroaryl, C₃₋₈-heterocyclyl and C₃₋₁₀-cycloalkyl can be optionally substituted with one or more substituents independently selected from hydroxy, sulfanyl, sulfo, oxo, halogen, amino, cyano, nitro, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, amino, C₁₋₆-alkyl, C₂₋₆-alkenyl, aryl, heteroaryl, C₃₋₈-heterocyclyl and C₃₋₁₀-cycloalkyl can be optionally substituted with one or more substituents independently selected from hydroxy, sulfanyl, sulfo, oxo, halogen, amino, cyano, nitro, C₁₋₆-alkyl, C₂₋₆-alkenyl, perhalomethyl and perhalomethoxy; and wherein R² can be optionally covalently bound to R¹ by an ether, thioether, C—C or C—N bond, to form a ring system with the N-atom to which R¹ and R² can be bound; and X is O or S; and L is a group such that —(C═X)-L is a hydrolysable group.

In one embodiment, the compound is of the formula:

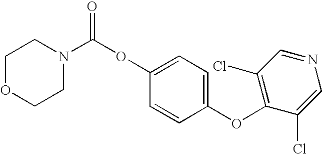

Additional compounds which may be used according to the invention are disclosed in U.S. Patent Application Publication No.: 20030166644. Such compounds include compounds of the formula:

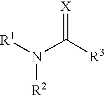

wherein R¹ can be selected from C1-6-alkyl, C2-6-alkenyl and C3-10-cycloalkyl, each of which is optionally substituted with one or more substituents independently selected from hydroxy, sulfanyl, oxo, halogen, amino, cyano and nitro; and R² is selected from C1-6-alkyl, C2-6-alkenyl, aryl, heteroaryl, C3-8-heterocyclyl and C3-10-cycloalkyl, each of which is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8 heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, perhalomethyl and perhalomethoxy; and wherein R² is optionally covalently bound to R¹ by an ether, thioether, C—C or C—N bond, to form a ring system with the N-atom to which R¹ and R² are bound; and R³ is selected from hydroxy, sulfanyl, sulfo, amino, C1-6-alkyl, C2-6-alkenyl, aryl, heteroaryl, C3-8-heterocyclyl and C3-10-cycloalkyl, each of which is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, aryl, heteroaryl, C3-8-heterocyclyl and C3-10-cycloalkyl, wherein each of hydroxy, sulfanyl, sulfo, amino, C1-6-alkyl, C2-6-alkenyl, aryl, heteroaryl, C3-8-heterocyclyl and C3-10-cycloalkyl is optionally substituted with one or more substituents independently selected from hydroxy, sulfanyl, sulfo, oxo, halogen, amino, cyano, nitro, C1-6-alkyl, C2-6-alkenyl, perhalomethyl and perhalomethoxy; and X is O or S.

Other suitable lipase inhibitors which may be used according to the invention include compounds of the formula:

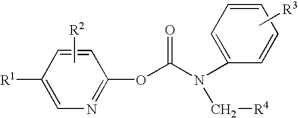

wherein R¹ and R² can be substituted or unsubstituted amide or thioamide, urea or thiourea, benzamide or alkoxy; and R¹-R⁴ can independently be H, halo, substituted or unsubstituted OH, sulfanyl, amino, sulfo, alkyl or cycloalkyl, alkenyl, aryl or heteroaryl or heterocyclyl. See PCT Patent Application Publication No. 2004/111031 for these and other lipase inhibitors which may be used according to the invention.

Other suitable lipase inhibitors which may be used according to the invention are piperazine carbamates of the formula:

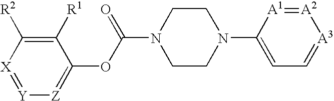

wherein X is N or CR³; Y is N or CR⁴; Z is N or CR⁵; A¹ is N or CR⁶, A² is N or CR⁷, A³ is N or CR⁸; provided that at least one of A¹, A² and A³ is N; and R¹-R⁸ is H, halo, (un)substituted OH, sulfanyl, amino, sulfo, alkenyl, (hetero)aryl, (cyclo)alkyl or heterocyclyl. In some embodiments, A¹ and Z are each N and A² and A³ are CH and R¹ and R² are H and X is CR³. In further embodiments, R³ is

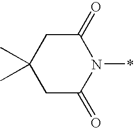

Other suitable lipase inhibitors which may be used according to the invention are disclosed in PCT International Patent Application No. WO 2004/111004 which is herein incorporated by reference in its entirety. These inhibitors are of the formula:

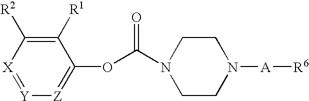

wherein R¹ and R² are each independently be H, amino, halo, substituted or unsubstituted OH, sulfanyl, sulfo, alkoxy, alkenyl, aryl, heteroaryl, alkyl, cycloalkyl, or heterocyclyl; X can be N or CR³, Y can be N or CR⁴, Z can be N or CR⁵; provided that X, Y and Z are not all CH; and R³-R⁵ can each independently be selected from H, F, substituted or unsubstituted OH, amino, sulfanyl, sulfo, alkenyl, aryl or heteroaryl, alkyl or cycloalkyl or heterocyclyl; and R⁶ can be substituted or unsubstituted alkenyl, aryl or heteroaryl, alkyl or cycloalkyl or heterocyclyl; and A can be CH₂, or CH₂CH₂.

Other suitable inhibitors can include carbazates as taught by De Jong et al., Bioorganic & Medicinal Chemistry Letters 14(7):1741-1744 (2004).

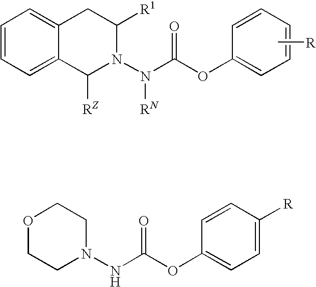

These compounds include those of the above formula as described therein.

Carbamoyl-Triazole Based Inhibitors of the following formula can also be useful as MGL inhibitors according to the invention. These include compounds of the formula:

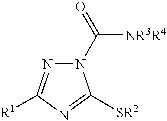

in which R¹ can be aryl or substituted aryl (e.g., phenyl, 4-ClC₆H₄, 4-MeC₆H₄, PhCH:CH, 2-naphthyl,), and R² can be alkyl or substituted alkyl; and R³ and R⁴ can each be methyl; R³ can be substituted or unsubstituted phenyl and R⁴ can be methyl; or R³R⁴N can be morpholino or piperidino. See, Ebdrup et al., Journal of Medicinal Chemistry 47(2):400-410 (2004).

Also suitable lipase inhibitors may be of the following formula:

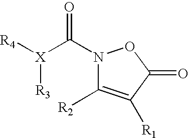

particularly as taught by Lowe et al., Bioorganic & Medicinal Chemistry Letters 14(12):3155-3158 (2004). In the above formula, X can be N or C, R₁ can be unsubstituted or substituted thioaryl with up to three aryl substituents selected independently from halogen (e.g., F, Cl, Br), (C1 to C6)alkyl, (C1 to C6)alkoxy, cyano, and nitro; trifluoromethyl, and R₂ can be hydrogen or substituted or unsubstituted (C1 to C6)alkyl (e.g., methyl, ethyl, propyl). R₃ and R₄ are independently selected from substituted or unsubstituted alkyl, heteroalkyl, aryl, heteroaryl, cycloalkyl, alkylaryl, alkylheteroaryl, alkylcycloalkyl. For instance, the R₃ and R₄ members can independently selected from quinolinyl, —CH2-cyclohexyl, benzyl, (2-, or 3-fluorobenzyl), furanyl, —CH2-furanyl, thienyl, —CH2-thienyl, —CH2CH2Ph, —CH2CH2(indolyl), phenyl, chlorophenyl (e.g., 4-chlorophenyl), pyridyl (e.g., 2-pyridyl), methyl, ethyl, and isopropyl. In some embodiments of each of the above, X is N. In further embodiments, particularly wherein X is N, R3 and R4, together with the nitrogen to which they are attached, form a substituted or unsubstituted cyclic amine which can, for instance, be a pyrrolidine, piperidine, N-benzylpiperazine, N-phenylpiperazine, N-(2-chlorophenyl)piperazine, N-(3-methoxyphenyl)piperiazine, N-(2-pyrimidinyl)piperazine, morpholine, thiomorpholine, homopiperidine,) 3-pyrroline, or indoline. Substituents for the cyclic amine can be substituted or unsubstituted (C1-C6)alkyl, or (C12-C6)alkoxy, trifluoromethyl, halogen, aryl, heteroaryl, alkyl aryl, alkoxyaryl, alkoxyheteroaryl, or alkyl heteroaryl. Substituents accordingly include, but are not limited to, methyl, methoxy, ethyl, ethoxy, benzyl (e.g., 4-benzyl), phenoxy, phenyl (e.g., 4-phenyl), 4-(3-fluorophenyl), 4-(3-methyl)phenyl, 4-(4-methyl)phenyl), 4-(4-CF₃-phenyl); 4-(4-tert-butylphenyl), hydroxy (e.g., 4-OH), keto (4-keto), 4-(4-CH3-2-isoxazoyl), 4-(5-CH3-2-oxodiazolyl), 3,5, dialkyl (e.g., dimethyl); and 3,3, dialkyl (e.g., dimethyl), and C(O)Oalkyl (e.g, 3C(O)OEt).
In some embodiments, the compound is of the formula:

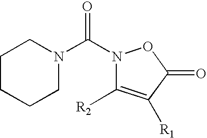

wherein R₂ can be hydrogen or (C1 to C6)alkyl (e.g., methyl, ethyl, propyl) and R₁ can be substituted or unsubstituted aryl or heteroaryl, or substituted or unsubstituted alkyl. In some embodiments, R₁ can be S-4-chlorophenyl, SO2-(4-Chloro)phenyl, S-phenyl, benzyl,), (C1-C10)alkyl (e.g., methyl, ethyl, isopropyl, butyl, isobutyl, n-butyl, t-butyl), cyclo(C4-C10)alkyl (e.g., cyclopentyl, cyclohexyl), 4-tetrahydropyranyl, 4-tetrahydrothiopyranyl, or 4-tetrahydrothiopyranyl dioxide. In other embodiments, R₁ can be substituted or unsubstituted phenyl, naphthyl, or indolyl, or heteroalkyl (e.g., —CH2CH2OCH3).

### Oxadiazolinones

3-phenyl-5-alkoxy-1,3,4-oxadiazol-2-ones as lipase inhibitors may also be used according to the invention. Such compounds are taught in U.S. Pat. No. 6,369,088 which is incorporated herein by reference. These compounds are of the general formula:

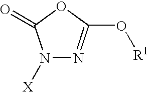

in which X can be a substituted ary or heteroaryl member and R¹ alkyl or cycloalkyl. Other lipase inhibitors which may be used according to the invention include compounds of the following formula:

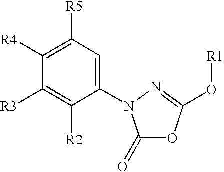

in which: R¹ is C₁-C₆-alkyl or C₃-C₉-cycloalkyl, wherein both groups are optionally substituted one or more times by phenyl, C₁-C₄-alkyloxy, S—C₁-C₄-alkyl, N(C₁-C₄-alkyl)₂, and wherein phenyl is optionally substituted one or more times by halogen, C₁-C₄-alkyl, C₁-C₄-alkyloxy, nitro, or CF₃; and R², R³, R⁴ and R⁵ independently hydrogen, halogen, nitro, C₁-C₄-alkyl, C₁-C₉-alkyloxy; C₆-C₁₀-aryl-C₁-C₄-alkyloxy, C₆-C₁₀-aryloxy, C₆-C₁₀-aryl, C₃-C₈-cycloalkyl or O—C₃-C₈-cycloalkyl, each of which is optionally substituted once, twice or three times by halogen, CF₃, C₁-C₄-alkyloxy or C₁-C₄-alkyl; 2-oxopyrrolidin-1-yl, 2,5-dimethylpyrrol-1-yl or NR⁶-A-R⁷, with the proviso that R², R³, R⁴ and R⁵ are not simultaneously hydrogen, and at least one of the radicals R², R³, R⁴ or R⁵ is the radical 2-oxopyrrolidin-1-yl, 2,5-dimethylpyrrol-1-yl, or NR⁶-A-R⁷, and wherein: R⁶ is hydrogen, C₁-C₄-alkyl or C₆-C₁₀-aryl-C₁-C₄-alkyl, where aryl may be substituted by halogen, CF₃, C₁-C₈-alkyloxy or C₁-C₄-alkyl; A is a single bond, COₙ, SOₙ, or CONH; n is 1 or 2; R⁷ is hydrogen; C₁-C₁8-alkyl or C₂-C₁8-alkenyl, wherein C₁-C₁₈-alkyl or C₂-C₁₈-alkenyl are optionally substituted one to three times by C₁-C₄-alkyl, halogen, CF₃, C₁-C₄-alkyloxy, N(C₁-C₄-alkyl)₂, —COOH, C₁-C₄-alkyloxycarbonyl, C₆-C₁₂-aryl, C₆-C₁₂-aryloxy, C₆-C₁₂-arylcarbonyl, C₆-C₁₀-aryl-C₁-C₄-alkyloxy or oxo, wherein aryl is in turn optionally substituted by halogen, C₁-C₄-alkyl, aminosulfonyl or methylmercapto; C₆-C₁₀-aryl-C₁-C₄-alkyl, C₅-C₈-cycloalkyl-C₁-C₄-alkyl, C₅-C.₈-cycloalkyl, C₆-C₁₀-aryl-C₂-C₆-alkenyl, C₆-C₁₀-aryl, biphenylyl, biphenylyl-C₁-C₄-alkyl, indanyl, each of which is optionally substituted once or twice by C₁-C₁₈-alkyl, C₁-C₁₈-alkyloxy, C₃-C-cycloalkyl, COOH, hydroxyl, C₁-C₄-alkylcarbonyl, C₆-C₁₀-aryl-C₁-C₄-alkyl, C₆-_{C10}-aryl-C₁-C₄-alkyloxy, C₆-C₁₀-aryloxy, nitro, cyano, C₆-C₁₀-aryl, fluorosulfonyl, C₁-C₆-alkyloxycarbonyl, C₆-C₁₀-arylsulfonyloxy, pyridyl, NHSO₂—C₆-C₁₀-aryl, halogen, CF₃ or OCF₃, wherein alkyl is in turn optionally substituted by C₁-C₄-alkyloxycarbonyl, CF₃ or carboxyl, and aryl is also optionally substituted by halogen, CF₃ or C₁-C₄-alkyloxy; or the group Het-(CH₂)ᵣ—, wherein r=0, 1, 2 or 3 and Het=a saturated or unsaturated 5-7-membered heterocycle, optionally benzo-fused and optionally substituted by C₁-C₄-alkyl, C₆-C₁₀-aryl, halogen, C₁-C₄-alkyloxy, C₁-C₄-alkyloxycarbonyl, C₆-C₁₀-aryl-C₁-C₄-alkyl, C₆-C₁0-aryl-C₁-C₄-alkylmercapto or nitro, wherein the benzo-fused aryl is in turn optionally substituted by halogen, C₁-C₄-alkyloxy or CF₃ and the alkyl in arylalkyl is also optionally substituted by methoxy and CF₃. In some embodiments, R¹ is C₁-C₄-alkyl.

### Benzotriazoles

Benzotriazole inhibitors may also be used according to the invention as MGL inhibitors. Such compounds are taught in PCT International Patent Application Publication No. WO 2004/0127484 which is incorporated herein by reference. For instance, compounds of the following formula may be used as lipase inhibitors according to the invention:

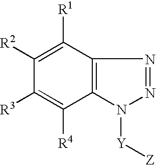

In the above formula, R¹ to R⁴ can each be independently H, alkyl, alkoxy, aryl, aryloxy; Y can be CO, CS, CH₂; Z can be OR⁵, NR⁵R⁶, or (O—, N— interrupted) (substituted) or Q¹, wherein Q¹ is a moiety of the formula:

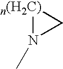

in which n is an integer from 1-5; and R⁵, R⁶ are independently alkyl, alkenyl, alkoxy, aryl, phenylalkyl, or aryloxy.

Additional such compounds can include benzotriazoles of formula:

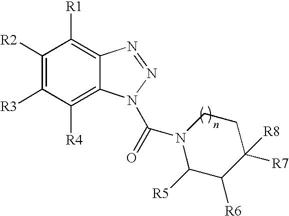

in which: n is an integer selected from 0, 1 and 2 R¹ to R⁸ are each H, except that one of R² and R³ may be selected from Br, Cl, CH₃, CN, NH₂, NO₂, CF₃, OCH₃, phenoxy, benzoyl, CH(OH)-phenyl, S-cyclohexyl, and CO—OCH₃; or R¹ is Cl and R³ is CF₃; or R² is F and R³ is Cl; or one of R⁶ and R⁷ may be selected as follows: R⁶ is CH₃ or R⁷ is selected from CH₃, C₂H₅, CH(CH₃)₂, C(CH₃)₃, CF₃, Br, Cl, benzyl and CO—OC₂H₅; or R⁶ and R⁷ each are CH₃; or R⁶ and R⁷ may be replaced by a double bond between the ring carbon to which they are attached; or R⁵ and R⁶ or R⁶ and R⁷ may, in combination with the carbon to which they are attached, form a benzo-fused ring or, when n is 0, may form cyclohexanediyl, the ring formed from R⁶, R⁷ and the carbons to which they are attached being optionally substituted singly by NH₂ or NO₂ or substituted singly or doubly by OCH₃; and R⁷ and R⁸, together with the carbon to which they are attached, may form cyclopentyl, diazirinediazirine or .═CH₂; provided, however, that when R¹ to R⁵ and R⁸ are H, and R⁶, R⁷ and the carbons to which they are attached form a benzo-fused ring, n is not 1, and when R¹ and R³-R⁸ are H and R² is CH₃, n is not 1.

### Oxobenzisothiazole or -Oxazole Carboxamides

Additional lipase inhibitors which may be used according to the invention include carboxamides of the general formula:

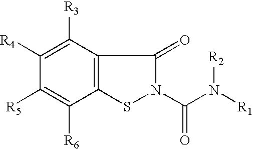

in which R¹ can be selected from alkyl, haloalkyl, alkenyl, alkynyl, alkylcycloalkyl, cycloalkyl, substituted or unsubstituted alkylheterocyclyl, and aryl; and R² can be H or alkyl; and R³ to R⁶ can independently be H, substituted or unsubstituted alkyl, haloalkyl, alkoxyalkyl, thioalkyl, hydroxy, alkenyl, alkynyl, alkylaryl, alkylcycloalkyl, alkylheterocyclyl, alkylcarbonyl, alkoxycarbonyl, phenyl or aryl. See, PCT International Patent Application Publication No. WO2004094394 which is incorporated herein by reference.

3-Oxo-3H-benzo[d]isoxazole carboxamides may also be useful as lipase inhibitors for use according to the invention (see, PCT International Application Publication No. WO 2004/094393). Thus, lipase inhibitors of the following formula are also contemplated for use according to the invention:

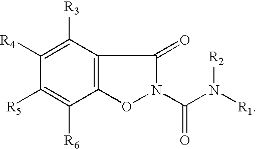

In the above formula, R¹ can be H, substituted or unsubstituted alkyl, cycloalkyl, alkenyl, alkynyl, cycloalkenyl or cylcloalkynyl, alkylamine, aryl or a heterocycle; R² can be H; and R³-R⁶ can independently be H, halogen (halo), hydroxy (OH), amino, substituted or unsubstituted alkyl, cycloalkyl, alkenyl, alkynyl, cycloalkenyl or cylcloalkynyl, an amide, aryl or heterocycle. In one embodiment, R³ to R⁶ are each H. In another embodiment, R²-R⁶ are each H.

### Glycerides, Phospholipides, and PAF Antagonists

Optically active C-analogs of glycerides, phospholipides, and PAF antagonists may also be used as lipase inhibitors according to the invention. Such compounds are of the formula:

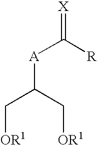

in which R can be a C₁₋₂₁ saturated or unsaturated aliphatic group group; R¹ can be H, R²CO, or OP(O)(OY¹)(OY²); wherein R² can be C₁₋₂₁ a saturated or unsaturated aliphatic group; X can be O or CH₂, and wherein Y¹ can be H, or a pharmacologically acceptable counter ion including, for instance, Na, K, CH₂CH₂N⁺Me₃; and Y² can be H or a pharmacologically acceptable counter ion including, for instance, Na or K, and A can be a methylene chain from 1 to 6 carbons in length. See German Patent No. DE4329069.
Oxetan-2-ones

In another embodiment, the lipase inhibitor may be a 4-(acyloxyethyl)oxetan-2-ones of the formula:

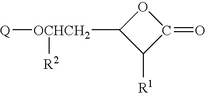

wherein Q is a group having the formula (R³R⁴)NCO(X)ₙ—CO— and R¹ and R² each are independently alkyl with up to 18 C atoms substituted by 1 to 3 halogen atoms or alkyl, alkenyl, alkynyl or alkadienyl groups with up to 20 C atoms optionally interrupted by a 1,4-arylene group, optionally substituted by an aryl group in the omega-position and optionally substituted by an aryl-C1-4-alkyl group, whereby R¹ can be interrupted by an O or S atom or by a sulphinyl or sulphonyl group in a position other than the .alpha.-position to an unsaturated C atom, or R¹ is an aryl-NH— or aryl-C1-4-alkyl-OCONH— group, R³ and R⁴ each are independently hydrogen or C1-4-alkyl or together with the N atom to which they are attached form a saturated 3- to 6-membered ring optionally containing an O or S atom in a position other than the alpha.-position to the N atom, n is the number 1 or 0, X is an alkylene group, which contains up to 8 C atoms, which is optionally interrupted by an O or S atom or by a sulphinyl or sulphonyl group and which is optionally substituted by a hydroxy, mercapto, aryl, aryloxy, arylthio, aryl-C1-4-alkyl, aryl-C1-4-alkoxy, aryl-C1-4-alkylthio, aryl-C1-4-alkylidene, C3-7-cycloalkylidene or C1-6-alkylidene group or by one or two C1-6-alkyl, C1-6-alkoxy or C1-6-alkylthio groups, whereby two C1-6-alkyl, C1-6-alkoxy or C1-6-alkylthio groups on the same C atom or on two adjacent C atoms can form an optionally mono-unsaturated 3- to 7-membered ring and an optionally present hydroxy or mercapto group or an optionally present unsaturated C atom must be in a position other than the alpha-position to an optionally present O or S atom or to an optionally present sulphinyl or sulphonyl group, or X is a group of the formula ═CHN(R,R⁰) where R and R⁰ each are independently hydrogen C1-4-alkyl, C1-4-alkyl(CO or OCO)—, aryl, aryl(CO or OCO)—, aryl-C1-4-alkyl or aryl-C1-4-alkyl(CO or OCO)—. See U.S. Pat. No. 5,260,310 which discloses these and other lipase inhibitors which may be of use according to the invention.

In other embodiments, the compound may be of the formula:

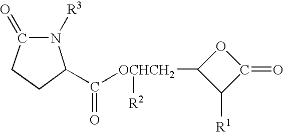

wherein R¹ and R² each are independently alkyl with up to 18 C atoms substituted by 1 to 3 halogen atoms or alkyl, alkenyl, alkynyl or alkadienyl groups with up to 20 C atoms optionally interrupted by a 1,4-arylene group, optionally substituted by an aryl group in the omega-position and optionally substituted by an aryl-C1-4-alkyl group, whereby R¹ can be optionally interrupted by an O or S atoms or by a sulphinyl or sulphonyl group in a position other than the alpha-position to an unsaturated C atom, or R.¹ is an aryl-NH— or aryl-C1-4-alkyl-OCONH— group, and R³ is hydrogen or C1-4-alkyl.

See U.S. Pat. No. 5,376,674 and European Patent Publication No. EP444482 which discloses these and other suitable lipase inhibitors which may be of use according to the invention.

In other embodiments, the lipase inhibitor may be a compound of the formula:

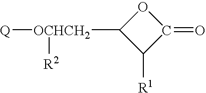

wherein Q is a group having the formula (R³R⁴)NCO—X¹- and R¹ and R² each are independently alkyl with up to 18 C atoms substituted by 1 to 3-halogen atoms or alkyl, alkenyl, alkynyl or alkadienyl groups with up to 20 C atoms optionally interrupted by a 1,4-arylene group, optionally substituted by an aryl group in the omega-position and optionally substituted by an aryl-C1-4-alkyl group, whereby R¹ can optionally be interrupted by an O or S atom or by a sulphinyl or sulphonyl group in a position other than the alpha-position to an unsaturated C atom, or R¹ is an aryl-NH— or aryl-C1-4-alkyl-OCONH-group, R³ and R⁴ each are independently hydrogen or C1-4-alkyl or together with the N atom to which they are attached form a unsubstituted saturated 3- to 6-membered ring optionally containing an O or S atom in a position other than the alpha-position to the N atom, and X′ is an alkylene group containing up to 6 C atoms which can be substituted by a C1-4-alkoxy, aryl, aryloxy, arylthio, aryl-C1-4-alkyl, aryl-C1-4-alkoxy or aryl-C1-4-alkylthio group or by one or two C1-6-alkyl groups, whereby two C1-6-alkyl groups attached to adjacent C atoms can form a 3- to 7-membered ring.

In other embodiments of the above, R¹ and R² each are C1-20-alkyl, R³ and R⁴ each are hydrogen and X′ is an alkylene group containing up to 6 C atoms which can be substituted by a C1-4-alkoxy group or by one or two C1-6-alkyl groups, whereby two C1-6-alkyl groups attached to adjacent C atoms can form a 3- to 7-membered ring. In still other embodiments of the above, R¹ is hexyl, R² is undecyl and X′ is ethylene, 1-methoxy-1,2-ethylene or 1,2-cyclohexylene. See U.S. Pat. No. 5,466,708 which discloses the above and other compounds which may be used according to the invention.

In other embodiments of the above, the compound is of the formula

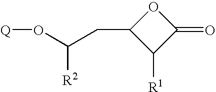

in which Q is R³R⁴NCOXₙCO, R³R⁴NCOX¹ or Q¹:

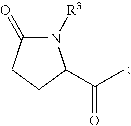

and wherein R¹ and R² can independently be haloalkyl, optionally 1,4-arylene-interrupted or optionally aryl-substituted alkyl, alkenyl, and alkynyl; and R³ and R⁴ can independently be H or alkyl; R³R⁴N can be a saturated 3-6 membered ring; and n can be 0 or 1; and X can be optionally O, S, SO, or SO₂— interrupted, substituted or unsubstituted alkylene, or aminomethylene; and X¹ can be a substituted or unsubstituted alkylene.

### Triacylglycerol Analogues

In other embodiments, a lipase inhibitor for use according to the invention may be a bis-2-oxo amide triacylglycerol analogue as taught by Kotsovolou, et al., Journal of Organic Chemistry 66(3):962-967 (2001). In some embodiments, the triacylglycerol analog is 2-[(2-oxohexadecanoyl)amino]-1-[[(2-oxohexadecanoyl)amino]methyl]ethyl decanoate. In further embodiments, the acyl side chains may be saturated or unsaturated and be from 6 to 21, or 10 to 18, carbon atoms in length. For example:

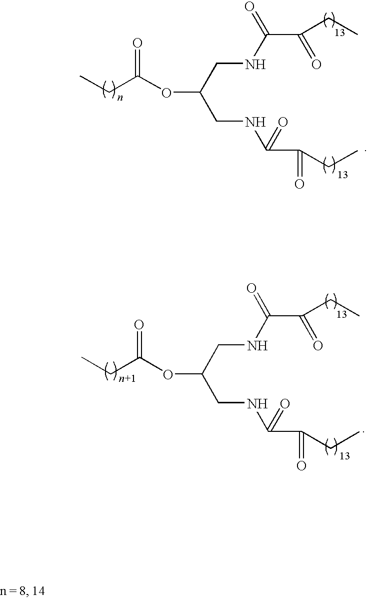

### Isocoumarins

In other embodiments, the compound for use according to the invention is an isocoumarin of the general formula:

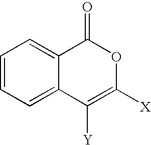

in which X and Y may independently be halo, nitro, or cyano, or substituted or unsubstituted alkoxy, and substituted or unsubstituted mono- or di-alkylamino, or amino. Particularly preferred are substituted or unsubstituted 3-alkoxy-4-chloroisocoumarins, 3-alkoxy-4-chloro-7-nitroisocoumarins, and 3-alkoxy-7-amino-4-chloroisocoumarins. In one embodiment the inhibiboris a 7-amino-4-chloro-3-ethoxyisocoumarin and 4-chloro-3-ethoxyisocoumarin. See, Harper J. W, et al. J.C. Biochemistry 24, 7200 (1985),

### Assay Methods for Measuring MGL Inhibition

Methods of measuring inhibition of MGL are known to one of ordinary skill in the art. For instance, an enzyme assay for measuring MGL activity is taught by Dinh et al., PNAS, 99(16):10819-10824 (2002). The ability of an agent to inhibit the activity can be determined by conducting the assay in the presence and absence of the agent and determining if the agent inhibits the activity of the enzyme. In a preferred embodiment, the assay uses 2-AG as the substrate. The substrate may be isotopically or otherwise labeled to facilitate its detection or the detection of its reaction products. Loss of substrate or formation of products may be monitored. The substrate may be labeled 2-arachidonyl glycerol and the measured reaction products may be labeled arachidonic acid or labeled glycerol. A partially purified preparation of MGL suitable for MGL assaying its activity may be obtained by centrifugation of mammalian brain tissue homogenates and isolating the cytosolic fractions enriched in MGL activity is also taught by Dinh et al., PNAS, 99(16): 10819-10824 (2002). This reference is incorporated by reference in its entirety herein and with particular reference to methods of obtaining MGL and assaying MGL activity). Partially purified MGL may also be obtained by ultracentrifugation of extracts of mammalian cells which express MGL or by recombinant cell systems in recombinant bacteria, recombinant yeast or other cultured cells in which mammalian brain MGL is expressed as described, for instance, by Dinh et al., Mol. Pharm. 66(5): 1260-4 (2004). Purified MGL may be obtained by overexpression in E. Coli bacteria of the mammalian MGL gene modified in such a way as to contain sequences of nucleotides encoding for a short series of histidine residues (histidine tag methods) and by fractionating extracts of the recombinant E. Coli over appropriate chromatographic columns containing ligated cobalt or Ni ions.

### FAAH Inhibitors

In all such above aspects of the invention, and embodiments thereof, setting forth a FAAH inhibitor, in one further embodiment the FAAH inhibitor is Compound M, URB597 and AM374 or a haloenol lactone as taught in U.S. Pat. No. 6,525,090 which is incorporated herein by reference.

Trifluoromethylketone inhibitors such as the compound of Formula IX are also contemplated for use in inhibiting FAAH to raise endogenous levels of anandamide or treat the subject conditions and disorders.

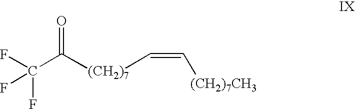

Such compounds are taught in U.S. Pat. No. 6,096,784 herein incorporated by reference.

Other compounds for use according to the invention include octylsulfonyl and octylphosphonyl compounds. See, Quistand, et al., in Toxicology and Applied Pharmacology, 179:57-63 (2002). See also Quistand, et al., in Toxicology and Applied Pharmacology, 173:48-55 (2001).

Other compounds for use according to the invention include the alpha-keto-oxazolpyridines which are reversible and extremely potent inhibitors of FAAH. See, Boger et al., PNAS USA, 97:5044-49 (2000). Suitable compounds include compounds of the Formula:

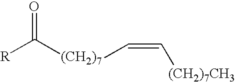

wherein R is an alpha-keto oxazolopyridinyl moiety such as

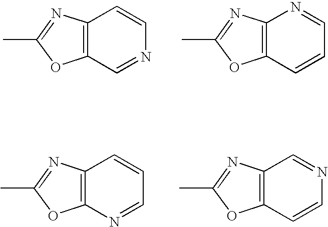

Boger et al. teach other suitable compounds for use according to the invention including substituted alpha-keto-heterocycle analogs of fatty acid amides. In particular, wherein R is an alpha-keto oxazolopyridinyl moiety and the fatty acid moiety is a homolog of oleic acid or arachidonic acid.

Other FAAH inhibitors for use according to the invention include fatty acid sulfonyl fluorides such as compound AM374 which irreversibly binds FAAH. See, Deutsch, et al., Biochem. Biophys Res Commun., 231:217-221 (1997).

Other preferred FAAH inhibitors include, but are not limited to, the carbamate FAAH inhibitors disclosed in Kathuria et al., Nat Med Jan; 9(1):76-81 (2003) incorporated herein by reference for the FAAH inhibitor compounds it discloses. Particularly preferred are selective FAAH inhibitors such as URB532 and URB597 disclosed therein.

FAAH inhibitors for use according to the invention include compounds of the following formula which inhibit FAAH:

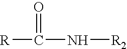

In the above formula, R is a polyunsaturated, substituted or unsubstituted hydrocarbyl group, wherein the hydrocarbyl group has from 18 to 22 carbon atoms; and R₂ is selected independently from substituted or unsubstituted cycloalkyl (C3-6) group and substituted or unsubstituted phenyl group. In some embodiments, the hydrocarbyl group R is a straight or branched chain C12-C26 fatty acid and may be saturated, monounsaturated, diunsaturated, or polyunsaturated.

In some embodiments, the fatty acid amide hydrolase inhibitor is selected from the group consisting of stearylsulfonyl fluoride, phenylmethylsulfonyl fluoride, trifluoromethyl ketones, diazomethylarachidonyl ketone, and pyrazinamide.

In some embodiments the FAAH inhibitor is represented by the following formula: A-B-C wherein: A is an α-keto heterocyclic pharmacophore for inhibiting the fatty acid amide hydrolase; B is a chain for linking A and C, said chain having a linear skeleton of between 3 and 9 atoms selected from the group consisting of carbon, oxygen, sulfur, and nitrogen, the linear skeleton having a first end and a second end, the first end being covalently bonded to the α-keto group of A, with the following proviso: if the first end of said chain is an α-carbon with respect to the α-keto group of A, then the α-carbon is optionally mono- or bis-functionalized with substituents selected from the group consisting of fluoro, chloro, hydroxyl, alkoxy, trifluoromethyl, and alkyl; and C is an activity enhancer for enhancing the inhibition activity of said α-keto heterocyclic pharmacophore, said activity enhancer having at least one π-unsaturation situated within a π-bond containing radical selected from a group consisting of aryl, alkenyl, alkynyl, and ring structures having at least one unsaturation, with or without one or more heteroatoms, said activity enhancer being covalently bonded to the second end of the linear skeleton of B, the π-unsaturation within the π-bond containing radical being separated from the α-keto group of A by a sequence of no less than 4 and no more than 9 atoms bonded sequentially to one another, inclusive of said linear skeleton (see, U.S. Patent Application Publication No. 20030092734, published on May 15, 2003, which is specifically incorporated herein by reference with respect to the FAAH inhibitors disclosed therein).

In other embodiments the FAAH inhibitor is an (oxime)carbamoyl fatty acid amide hydrolase inhibitor (see, U.S. Patent Application Publication No. 20030195226 which is specifically incorporated herein by reference and particularly with respect to the FAAH inhibitors disclosed therein). In particular embodiments, the FAAH inhibitor is selected from the group consisting of
pyridine-3-carbaldehyde, O-[[(4-undecyloxy-phenyl)amino]carbonyl]oxime;
pyridine-3-carbaldehyde, O-[[(4-nonyloxy-phenyl)amino]carbonyl]oxime;
4-fluorobenzaldehyde, O-[[(4-decyloxy-phenyl)amino]carbonyl]oxime;
4-fluorobenzaldehyde, O-[[(4-octyloxy-phenyl)amino]carbonyl]oxime;
benzaldehyde, O-[[(4-nonyloxy-phenyl)amino]carbonyl]oxime;
4-fluorobenzaldehyde, O-[[(4-nonyloxy-phenyl)amino]carbonyl]oxime;
3,4-difluorobenzaldehyde, O-[[(4-nonyloxy-phenyl)amino]carbonyl]oxime;
2,6-difluorobenzaldehyde, O-[[(4-nonyloxy-phenyl)amino]carbonyl]oxime;
2,4-difluorobenzaldehyde, O-[[(4-nonyloxy-phenyl)amino]carbonyl]oxime;
3-fluorobenzaldehyde, O-[[(4-nonyloxy-phenyl)amino]carbonyl]oxime;
pyridine-3-carbaldehyde, O-[[(4-nonyloxy-phenyl)amino]carbonyl]oxime;
benzaldehyde, O-[[(4-decyloxy-phenyl)amino]carbonyl]oxime;
pyridine-3-carbaldehyde, O-[[(4-decyloxy-phenyl)amino]carbonyl]oxime;
pyridine-3-carbaldehyde, O-[[(4-dodecyloxy-phenyl)amino]carbonyl]oxime;
benzaldehyde, O-[[(4-octyloxy-phenyl)amino]carbonyl]oxime;
2,3-difluorobenzaldehyde, O-[[(4-nonyloxy-phenyl)amino]carbonyl]oxime;
benzaldehyde, O-[[(4-undecyloxy-phenyl)amino]carbonyl]oxime;
2,4,5-trifluorobenzaldehyde, O-[[(4-nonyloxy phenyl)amino]carbonyl]oxime;
4-fluorobenzaldehyde, O-[[(4-phenoxyphenyl)amino]carbonyl]oxime;
benzaldehyde, O-[[(4-undecyloxy-phenyl)amino]carbonyl]oxime;
4-trifluoromethyl-benzaldehyde, O-[[(4-nonyloxy-phenyl)amino]carbon-yl]oxime;
benzaldehyde, O-[[(4-phenoxyphenyl)amino]carbonyl]oxime;
pyridine-3-carbaldehyde, O-[[(4-heptyloxy-phenyl)amino]carbonyl]oxime;
benzaldehyde, O-[[[4-(2-phenylethoxy)phenyl]amino]carbonyl]oxime;
2-fluoro-3-trifluoromethyl-benzaldehyde, O-[[(4-nonyloxy-phenyl)amino]carbonyl]oxime;
(4-undecyloxy-phenyl)-carbamic acid phenyl ester;
propan-2-one, O-[[(4-heptyloxy-phenyl)amino]carbonyl]oxime;
propan-2-one, O-[[(4-nonyloxy-phenyl)amino]carbonyl]oxime;
benzaldehyde, O-[[[4-(phenylmethoxy)phenyl]amino]carbonyl]oxime;
4-fluorobenzaldehyde, O-[[[4-(2-phenylethoxy)phenyl]amino]carbonyl]-oxime;
2-fluoro-5-trifluoromethyl-benzaldehyde, O-[[(4-nonyloxy-phenyl)amino]carbonyl]oxime;
4-fluorobenzaldehyde, O-[[[4-(phenylmethoxy)phenyl]amino]carbonyl]oxime;
3-pyridinecarboxaldehyde, O-[[(3-phenoxyphenyl)amino]carbonyl]oxime-;
4-fluorobenzaldehyde, O-[[[4-(3-phenylpropoxy)phenyl]amino]carbonyl-]oxime;
benzaldehyde, O-[[(3-phenoxyphenyl)amino]carbonyl]oxime;
4-fluorobenzaldehyde, O-[[(4-pentyloxy-phenyl)amino]carbonyl]oxime;
4-fluorobenzaldehyde, O-[[(4-butoxy-phenyl)amino]carbonyl]oxime;
pyridine-3-carbaldehyde, O-[[(4-heptyloxy phenyl)amino]carbonyl]oxime;
3-pyridinecarboxaldehyde, O-[[(4-phenoxyphenyl)amino]carbonyl]oxime-; benzaldehyde, O-[[[4-(3-phenylpropoxy)phenyl]amino]carbonyl]oxime;
4-fluorobenzaldehyde, O-[[(4-pentyloxy-phenyl)amino]carbonyl]oxime;
4-fluorobenzaldehyde, O-[[(4-dodecyloxy-phenyl)amino]carbonyl]oxime-;
propan-2-one, O-[[(4-decyloxy-phenyl)amino]carbonyl]oxime;
benzaldehyde, O-[[(4-dodecyloxy-phenyl)amino]carbonyl]oxime;
benzaldehyde, O-[[(4-pentyloxy-phenyl)amino]carbonyl]oxime;
2,4-difluorobenzaldehyde, benzaldehyde, O-[[(4-nonanoylamino-phenyl-amino]carbonyl]oxime;
4-fluorobenzaldehyde, O-[[(4-heptyloxy-phenyl)amino]carbonyl]oxime;
benzaldehyde, O-[[(4-pentyloxy-phenyl)amino]carbonyl]oxime;
propan-2-one, O-[[(4-undecyloxy-phenyl)amino]carbonyl]oxime;
propan-2-one, O-[[(4-dodecyloxy-phenyl)amino]carbonyl]oxime;
pyridine-3-carbaldehyde, O-[[(4-pentyloxy-phenyl)amino]carbonyl]oxime;
benzaldehyde, O-[[(4-propoxy-phenyl)amino]carbonyl]oxime;
benzaldehyde, O-[[(4-heptyloxy-phenyl)amino]carbonyl]oxime;
benzaldehyde, O-[[(4-butoxy-phenyl)amino]carbonyl]oxime;
benzaldehyde, O-[[(4-hexyloxy-phenyl)amino]carbonyl]oxime;
propan-2-one, O-[[(4-heptyloxy-phenyl)amino]carbonyl]oxime;
pyridine-3-carbaldehyde, O-[[(4-hexyloxy-phenyl)amino]carbonyl]oxime; and
pyridine-3-carbaldehyde, O-[[(4-butoxy-phenyl)amino]carbonyl]oxime.

Other FAAH inhibitors for use according to the invention are characterized by a carbamic template substituted with alkyl or aryl groups at their O- and N-termini. Most such compounds inhibit FAAH, but not several other serine hydrolases, with potencies that depend on the size and shape of the substituents. Preferred compounds have a lipophilic N-alkyl substituent (e.g., n-butyl or cyclohexyl or cyclopentyl) and a bent O-aryl substituent with a various number of selected groups independently present. N-alkylcarbamic acid O-biphenyl-3-yl esters, N-cyclohexylcarbamic acid 3′- or 4′-substituted biphenyl-3-yl esters, and N-cyclohexylcarbamic acid 2- or 4-substituted biphenyl-3-yl esters are exemplary such compounds. See Tarzia et al., J Med. Chem. 46(12):2352 (2003) and Mor et al., J Med. Chem. 47(21): 4998 (2004). Method of screening compounds for FAAH inhibitory activity are well known in the art.

In another embodiment, the FAAH inhibitor is a bisarylimidazolyl fatty acid amide hydrolase inhibitor as disclosed in U.S. Patent Application Publication No. 20020188009, published Dec. 12, 2002, which is specifically incorporated herein by reference and particularly with respect to the FAAH inhibitors disclosed therein). In some embodiments, the FAAH inhibitor is selected from the group consisting of [6-(2-Methyl-4,5-diphenyl-imidazol-1-yl)-hexyl]-carbamic acid 2-fluoro-phenyl ester; [6-(2-Ethyl-4,5-diphenyl-imidazol-1-yl)-hexyl]-car-bamic acid tert-butyl ester; 6-(2-Ethyl-4,5-diphenyl-imidazol-1-yl)-hexyl]-carbamic acid sec-butyl ester; [6-(2-Methyl-4,5-diphenyl-imidazol-1-yl)-hexyl]-carbamic acid benzyl ester; 2-Propanone,O-[6-(2-methyl-4,5-diphenyl-1H-imidazol-1-yl)hexyl]amino]carbonyl]oxime; [6-(2-Methyl-4,5-diphenyl-imi-dazol-1-yl)-hexyl]-carbamic acid methyl ester; 6-(2-Methyl-4,5-diphenyl-im-idazol-1-yl)-hexyl]-carbamic acid phenyl ester; [6-(2-Methyl-4,5-diphenyl-imidazol-1-yl)-hexyl]-carbamic acid 4-fluoro-phenyl ester; [6-(2-Methyl-4,5-diphenyl-imidazol-1-yl)-hexyl]-carbamic acid 2,4-difluoro-phenyl ester; [6-(2-Methyl-4,5-diphenyl-imidazol-1-yl)-hexyl-]-carbamic acid 4-chloro-phenyl ester; [6-(2-Methyl-4,5-diphenyl-imidazol-1-yl)-hexyl]-carbamic acid 4-methoxy-phenyl ester; [6-(2-Methyl-4,5-diphen-yl-imidazol-1-yl)-hexyl]-carbamic acid o-tolyl ester; [6-(2-Methyl-4,5-diphenyl-imidazol-1-yl)-hexyl]-carbamic acid 4-cyano-phenyl ester; [6-(2-Methyl-4,5-diphenyl-imidazol-1-yl)-hexyl]-carbamic acid 2,6-dimethoxy-phenyl ester; [6-(2-Methyl-4,5-diphenyl-imidazol-1-yl)-hexyl]-carbamic acid 2-methoxy-phenyl ester; [7-(2-Methyl-4,5-diphen-yl-imidazol-1-yl)-heptyl]-carbamic acid methyl ester; [7-(2-Methyl-4,5-diphenyl-imidazol-1-yl)-heptyl]-carbamic acid ethyl ester; [7-(2-Methyl-4,5-diphenyl-imidazol-1-yl)-heptyl]-carbamic acid phenyl ester; [7-(2-Methyl-4,5-diphenyl-imidazol-1-yl)-heptyl]-carbamic acid 4-fluoro-phenyl ester; [7-(2-Methyl-4,5-diphenyl-imidazol-1-yl)-hept-yl]-carbamic acid 2-fluoro-phenyl ester; [7-(2-Methyl-4,5-diphenyl-imidazo-l-1-yl)-heptyl]-carbamic acid 2,4-difluoro-phenyl ester; [7-(2-Methyl-4,5-diphenyl-imidazol-1-yl)-heptyl]-carbamic acid 4-chloro-phenyl ester; [7-(2-Methyl-4,5-diphenyl-imidazol-1-yl)-heptyl]-c-arbamic acid 4-methoxy-phenyl ester; [7-(2-Methyl-4,5-diphenyl-imidazol-1-yl)-heptyl]-carbamic acid o-tolyl ester; [7-(2-Methyl-4,5-diphenyl-imidazo-l-1-yl)-heptyl]-carbamic acid 4-cyano-phenyl ester; [7-(2-Methyl-4,5-diphenyl-imidazol-1-yl)-heptyl]-carbamic acid 2,6-dimethoxy-phenyl ester; [7-(2-Methyl-4,5-diphenyl-imidazol-1-yl)-hept-yl]-carbamic acid 2-methoxy-phenyl ester; [5-(2-Methyl-4,5-diphenyl-imidaz-ol-1-yl)-pentyl]-carbamic acid ethyl ester; [5-(2-Methyl-4,5-diphenyl-imid-azol-1-yl)-pentyl]-carbamic acid phenyl ester; [5-(2-Methyl-4,5-diphenyl-i-midazol-1-yl)-pentyl]-carbamic acid 4-fluoro-phenyl ester; [5-(2-Methyl-4,5-diphenyl-imidazol-1-yl)-pentyl]-carbamic acid 2,4-difluoro-phenyl ester; [5-(2-Methyl-4,5-diphenyl-imidazol-1-yl)-penty-l]-carbamic acid 2-fluoro-phenyl ester; [5-(2-Methyl-4,5-diphenyl-imidazol-1-yl)-pentyl]-carbamic acid 4-chloro-phenyl ester; [5-(2-Methyl-4,5-diphenyl-imidazol-1-yl)-pentyl]-carbamic acid 4-methoxy-phenyl ester; [5-(2-Methyl-4,5-diphenyl-imidazol-1-yl)-pentyl]-carbamic acid o-tolyl ester; [5-(2-Methyl-4,5-diphenyl-imidazol-1-yl)-pent-yl]-carbamic acid 4-cyano-phenyl ester; [5-(2-Methyl-4,5-diphenyl-imidazol-1-yl)-pentyl]-carbamic acid 2,6-dimethoxy-phenyl ester; [5-(2-Methyl-4,5-diphenyl-imidazol-1-yl)-pentyl]-carbamic acid 2-methoxy-phenyl ester; [6-(2-Methyl-4,5-diphenyl-imidazol-1-yl)-hexyl]-c-arbamic acid 3,4-difluoro-phenyl ester; {6-[4,5-Bis-(4-fluoro-phenyl)-2-me-thyl-imidazol-1-yl]-hexyl}-carbamic acid 2-fluoro-phenyl ester; {6-[4,5-Bis-(4-fluoro-phenyl)-2-methyl-imidazol-1-yl]-hexyl}-carbamic acid 2,6-difluoro-phenyl ester; [6-(2-Methyl-4,5-diphenyl-imidazol-1-yl)-hexyl]-carbamic acid ethyl ester; Benzaldehyde, 0-[6-(2-methyl-4,5-dipheny-l-1H-imidazol-1-yl)hexyl]amino]carbonyl]oxime; 4-Fluorobenzaldehyde,O-[6-(-2-methyl-4,5-diphenyl-1H-imidazol-1-yl)hexyl]amino]carbonyl]oxime; 2-Nitrobenzaldehye, O-[6-(2-methyl-4,5-diphenyl-1H-imidazol-1-yl)hexyl]am-ino]carbonyl]oxime; 3-Nitrobenzaldehyde, O-[6-(2-methyl-4,5-diphenyl-1H-im-idazol-1-yl)hexyl]amino]carbonyl]oxime; 4-Nitrobenzaldehyde, O-[6-(2-methyl-4,5-diphenyl-1H-imidazol-1-yl)hexyl]amino]carbonyl]oxime; 3-Pyridinecarboxaldehyde, O-[6-(2-methyl-4,5-diphenyl-1H-imidazol-1-yl)he-xyl]amino]carbonyl]oxime; {4-[2-(2-Methyl-4,5-diphenyl-imidazol-1-yl)-etho-xy]-phenyl}-carbamic acid 3,4-difluoro-phenyl ester; {4-[2-(2-Methyl-4,5-diphenyl-imidazol-1-yl)-ethoxy]-phenyl}-carbamic acid 4-chloro-phenyl ester; {4-[3-(2-Methyl-4,5-diphenyl-imidazol-1-yl)-propox-y]-phenyl}-carbamic acid 3,4-difluoro-phenyl ester; {4-[3-(2-Methyl-4,5-diphenyl-imidazol-1-yl)-propoxy]-phenyl}-carbamic acid 4-methoxy-phenyl ester; {4-[3-(2-Methyl-4,5-diphenyl-imidazol-1-yl)-propoxy]-phenyl}-carbamic acid 4-chloro-phenyl ester; {4-[3-(2-Methyl-4,5-diphenyl-imidazol-1-yl)-propoxy]-phenyl}-carbamic acid 2-methoxy-phenyl ester; {4-[3-(2-Methyl-4,5-diphenyl-imidazol-1-yl)-propoxy]-phenyl}-carbamic acid 3-chloro-phenyl ester; {4-[2-(2-Methyl-4,5-diphenyl-imidazol-1-yl)-ethoxy]-phenyl}-carbamic acid phenyl ester; {4-[2-(2-Methyl-4,5-diphenyl-imidazol-1-yl)-ethoxy]-phenyl}-carbamic acid 2-fluoro-phenyl ester; {4-[2-(2-Methyl-4,5-diphenyl-imidazo-l-1-yl)-ethoxy]-phenyl}-carbamic acid 4-fluoro-phenyl ester; {4-[3-(2-Methyl-4,5-diphenyl-imidazol-1-yl)-propoxy]-phenyl}-carbamic acid phenyl ester; {4-[2-(2-Methyl-4,5-diphenyl-imidazol-1-yl)-ethoxy]-ph-enyl}-carbamic acid 4-methoxy-phenyl ester; {4-[3-(2-Methyl-4,5-diphenyl-1-midazol-1-yl)-propoxy]-phenyl}-carbamic acid 2-fluoro-phenyl ester; {4-[3-(2-Methyl-4,5-diphenyl-imidazol-1-yl)-propoxy]-phenyl}-carbamic acid 2,6-difluoro-phenyl ester; {4-[2-(2-Methyl-4,5-diphenyl-imidazol-1-y-l)-ethoxy]-phenyl}-carbamic acid ethyl ester; [1-Methyl-6-(2-methyl-4,5-di-phenyl-imidazol-1-yl)-hexyl]-carbamic acid 2-fluoro-phenyl ester; [1-Ethyl-6-(2-methyl-4,5-diphenyl-imidazol-1-yl)-hexyl]-carbamic acid 2-fluoro-phenyl ester; [1-Isopropyl-6-(2-methyl-4,5-diphenyl-imidazol-1-y-l)-hexyl]-carbamic acid 2-fluoro-phenyl ester and [6-(2-Methyl-4,5-diphenyl-imidazol-1-yl)-1-phenyl-hexyl]-carbamic acid 2-fluoro-phenyl ester.

In another embodiment, the FAAH inhibitor is a haloenol lactone compound of the following formula:

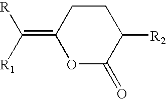

wherein R is hydrogen, R₁ is a halogen and R₂ is selected from the group consisting of aryl, aryloxy, and heteroaryl radicals. In one such embodiment, the haloenol lactone is E-6-(bromomethylene)tetrahydro-3-(1-naphthalenyl)-2H-pyrane-2-one. See U.S. Pat. No. 6,525,090 which is incorporated by reference in its entirety particularly with respect to the disclosure of such

### Methods of Screening Compounds for FAAH Inhibitory Activity.

While many FAAH inhibitory compounds are known in the art, additional suitable FAAH inhibitory compounds can be readily identified using methods known in the art. Methods for screening compounds for FAAH inhibitory activity in vitro are well known to one of ordinary skill in the art. Such methods are taught in Quistand, et al., in Toxicology and Applied Pharmacology, 179:57-63 (2002); Quistand, et al., in Toxicology and Applied Pharmacology, 173: 48-55 (2001); and Boger, et al., PNAS USA, 97:5044-49 (2000).

Methods for screening compounds for FAAH inhibitory activity in vivo and increased endogenous cannabinoid levels or activity are known to one of ordinary skill in the art. Such methods include measurement of fatty acid ethanolamides in tissue and are taught in Quistand, et al., in Toxicology and Applied Pharmacology, 179: 57-63 (2002); Quistand, et al., in Toxicology and Applied Pharmacology, 173: 48-55 (2001); Boger, et al., PNAS USA, 97:5044-49 (2000). See, U.S. Pat. No. 6,096,784. See also PCT Publication WO 98/24396. See, Cravatt, et al., PNAS, 98:9371-9376 (2001).

### Anandamide Transport Inhibitors

The anandamide transport inhibitors for use according to the invention include amide and ester analogs of anandamide and exhibit the tail, central and head pharmacophore portions represented by Structural Formula: X—Y-Z wherein the tail portion X is a fatty acid chain remnant, central portion Y is an amide or ester radical and head portion Z is selected form the group consisting of hydrogen, alkyl, hydroxy alkyl, aryl, hydroxy aryl, heterocyclic and hydroxy heterocyclic radicals. See U.S. Patent Application Publication No. 20030149082 published on Aug. 7, 2003 (Application Ser. No. 328742). U.S. Patent Application Publication No. 20030149082 is herein incorporated by reference in its entirety and in particular with respect to the anandamide transport inhibitors and anandamide transport inhibition assays disclosed therein.

Assays for anandamide transport inhibition are well known to one of ordinary skill in the art. Exemplary methods for screening such compounds and identifying novel suitable compounds with such inhibitory activity are taught in U.S. Patent Application Publication No. 20040048907 published on Mar. 11, 2004 (U.S. Pat. No. 439,347, filed May 15, 2003), PCT Patent Publication No. WO 03/097573, and U.S. Patent Application Publication No. 20030149082. Such assays can be used to identify other anandamide transport inhibitors for use according to the present invention. Exemplary anandamide transport inhibitors for use according to the invention include M404, AM1172, OMDM1 and UCM707. U.S. Patent Application Publication No. 20040048907 and PCT Patent Publication No. WO 03/097573 are herein incorporated by reference in their entirety and in particular with respect to the anandamide transport inhibitors and anandamide transport inhibition assays disclosed therein.

### CB1 Receptor Agonists for Use According to the Invention

A variety of CB1 receptor agonists are known to date; these include classical cannabinoids, such as, for example, Δ⁹-THC, non-classical cannabinoids, aminoalkylindoles and eicosanoids. The latter include the generally accepted endogenous CB1 receptor agonist anandamide. In all such above aspects of the invention, and embodiments thereof, setting forth a CB1 cannabinoid receptor agonist, in a further embodiment thereof, the CB1 cannabinoid receptor agonist is CP-55940, Win-55212-2, anandamide, methanandamide, or 2-arachidonoylglycerol.

CB1 Receptor Agonists for use according to the invention, include but are not limited to, compounds of Formula Ib as taught in U.S. Pat. No. 5,631,297.

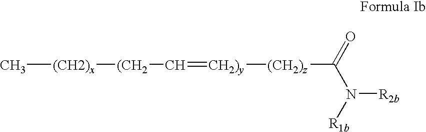

wherein R_{1b} and R_{2b} are each H or (CH₂)ₚ—(R_{4b} CH)_{q}—(CH₂)ᵣ—R_{3b}, wherein p, q and r are each an integer of from 0 to 10, preferably 1 to 4; and R_{3b} is OH, SH, CH₃, CH═CH₂,

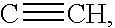

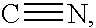

F, Cl, Br or I, preferably OH, SH or F, more preferably OH; R_{4b} is H or (CH₂)ₛCH₃, wherein s is an integer from 0 to 10, preferably 0 to 4; provided that p+q+r+s is less than or equal to 10, preferably less than or equal to 4, preferably one of R₁ₐ and R_{2b} is H and the other of R₁ₐ and R_{2b} is (CH₂)ₚ(R_{4b} CH)_{q}(CH₂)ᵣR_{3b};

x is an integer of from 0 to 18, preferably 2 to 5;

y is an integer of from 0 to 8, preferably 2 to 4; and

z is an integer of from 0 to 18, preferably 2 to 5.

Non-limiting examples of the compounds represented by Formula (Ib) which can be employed in the present invention include the following:
arachidonylethanolamide
arachidonylethanethiolamide
arachidonylfluoroethylamide
7,10,13,16-docosatetraenylethanolamide
arachidonylpropanolamide
8,11,14-eicosatrienylethanolamide
4,7,10,13,16,19-Docosahexaenylethainolamide
arachidylfluoroethylamide
arachidonylamide
arachidonyl-1-methyl-ethanolamide
arachidonyl-2-methyl-ethanolamide,
gamma-linolenylethanolamide,
linoleylethanolamide

In accordance with this aspect of the present invention, there are disclosed pharmaceutical compositions and methods for treating pain comprising use of direct acting cannabinoid receptor agonists (e.g., arachidonylethanolamide (anandamide), (R)-(+)arachidonyl-1-hydroxy-2-propylamide, cis-7,10,13,16-docosatetraenoylethanolamide, homo-delta-linoleyethanolamide, N-propyl-arachidonylethanolamide, N-ethyl-arachidonylethanolamide, and 2-arachidonylglycerol, and indirect acting FAAH inhibitors N-(4-hydroxyphenyl)-arachidonylamide, palmitylsulphonylfluoride, and arachidonyltrifluoromethylketone.

CB1 cannabinoid receptor agonists to be used according to the invention include those of the following formula:

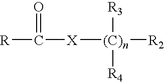

wherein X is N—R₁ or O; R is a saturated or unsaturated, chiral or achiral, cyclic or acyclic, substituted or unsubstituted hydrocarbyl group, wherein the hydrocarbyl group has 11 to 29 carbon atoms; R₁, R₃ and R₄ are selected independently from hydrogen, alkyl (C1-4), alkenyl (C2-4), alkynyl (C2-4), cycloalkyl (C3-6), or hydroxyalkyl group with from 2 to 4 carbon atoms; R₂ is OH or O—CO-alkyl, where the alkyl group has from 1 to 4 carbon atoms; and n is selected from 2 to 4.

### I. Cannabinoid Receptor Activity Screening.

While a great many CB1 agonist compounds are known in the art, additional suitable novel CB1 agonist compounds can be readily identified using methods known in the art. For instance, methods for screening compounds for CB1 agonist activity are well known to one of ordinary skill in the art. A variety of means may be used to screen cannabinoid CB1 receptor activity in order to identify the compounds for use according to the invention. A variety of such methods are taught in U.S. Pat. No. 5,747,524 and U.S. Pat. No. 6,017,919.

A. Ligand Binding Assays.

Ligand binding assays are well known to one of ordinary skill in the art. For instance, see, U.S. Patent Application No. US 2001/0053788 published on Dec. 20, 2001, U.S. Pat. No. 5,747,524, and U.S. Pat. No. 5,596,106 and (see, Felder, et al., Proc. Natl. Acad. Sci., 90:7656-7660 (1993)) each of which is incorporated herein by reference. The affinity of an agent for cannabinoid CB1 receptors can be determined using membrane preparations of Chinese hamster ovary (CHO) cells in which the human cannabis CB1 receptor is stably transfected in conjunction with [³H]CP-55,940 as radioligand. After incubation of a freshly prepared cell membrane preparation with the [³H]-ligand, with or without addition of compounds of the invention, separation of bound and free ligand can be performed by filtration over glass fiber filters. Radioactivity on the filter was measured by liquid scintillation counting.

The cannabinoid CB1 agonistic activity of a candidate compound for use according to the invention can also be determined by functional studies using CHO cells in which human cannabinoid CB1 receptors are stably expressed. Adenylyl cyclase can be stimulated using forskolin and measured by quantifying the amount of accumulated cyclic AMP. Concomitant activation of CB1 receptors by CB1 receptor agonists (e.g., CP-55,940 or (R)-WIN-55, 212-2) can attenuate the forskolin-induced accumulation of cAMP in a concentration-dependent manner. This CB1 receptor-mediated response can be antagonized by CB1 receptor antagonists. See, U.S. Patent Application No. US 2001/0053788 published on Dec. 20, 2001.

Samples rich in cannabinoid CB1 receptors and CB2 receptors, rat cerebellar membrane fraction and spleen cells can be respectively used (male SD rats, 7-9 weeks old). A sample (cerebellar membrane fraction: 50 μ.g/ml or spleen cells: 1(×10⁷ cells/ml), labeled ligand ([³H]Win55212-2, 2 nM) and unlabeled Win55212-2 or a test compound can be plated in round bottom 24 well plates, and incubated at 30° C. for 90 min in the case of cerebellar membrane fraction, and at 4° C. for 360 min in the case of spleen cells. As the assay buffer, 50 mM Tris solution containing 0.2% BSA can be used for cerebellar membrane fraction, and 50 mM Tris-HBSS containing 0.2% BSA can be used for spleen cells. After incubation, the samples are filtrated through a filter (Packard, Unifilter 24 GF/B) and dried. A scintillation solution (Packard, Microsint-20) can be added, and the radioactivity of the samples determined (Packard, Top count A9912V). The non-specific binding can be determined by adding an excess Win55212-2 (1 μM), and calculating specific binding by subtracting non-specific binding from the total binding obtained by adding the labeled ligand alone. The test compounds can be dissolved in DMSO to the final concentration of DMSO of 0.1%. EC₅₀ can be determined from the proportion of the specifically-bound test compounds, and the Kᵢ value of the test compounds can be calculated from EC₅₀ and K_{d} value of [³H]WIN55212-2. See, U.S. Pat. No. 6,017,919.

In one embodiment, the EC₅₀ for cannabinoid receptor binding is determined according to the method of Devane, et al., Science, 258: 1946-1949 (1992) and Devane, et al., J. Med. Chem., 35:2065 (1992). In this method, the ability of a compound to competitively inhibit the binding of a radiolabeled probe (e.g., ³H—HU-2430) is determined.

In other embodiments, the EC₅₀ of an agonist for the CB1 receptor is determined according to any one of the above ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

B. Effect on N-Type Calcium Channel Currents.

Cannabinoid agonist activity can also be assessed by studying activation of the signal transduction pathway of the CB1 receptor, but in addition, effect other nerve cell organelles under control of the CB1 signaling pathway in vitro. Specifically, the agonists can close the N-type calcium channels (see, Mackie, K. and Hille, B., Proc. Natl. Acad. Sci., 89:3825-3829 (1992)). See, U.S. Pat. No. 5,596,106 which is incorporated herein by reference which teaches how to identify CB1 agonists on nerve cells by measuring current flow using a whole-cell voltage-clamp technique. A cannabinoid agonist (e.g., anandamide or WIN 55,212 will inhibit the N-type calcium channel via the CB1 receptor, thus decreasing the current to the voltage clamp of −65 pA. The addition of an CB1 receptor antagonist will oppose the action of the agonist.

C. Cannabinoid CB2 Receptor Binding Assay.

A variety of means may be used to screen cannabinoid CB2 receptor activity in order to identify compounds for use according to the invention. Methods of studying CB2 receptor binding are well known to one of ordinary skill in the art. For instance, binding to the human cannabinoid CB2 receptor can be assessed using the procedure of Showalter, et al., J. Pharmacol Exp Ther., 278(3):989-99 (1996)), with minor modifications as taught for instance in U.S. Patent Application No. 20020026050, published Feb. 28, 2002. Each of which is incorporated herein by reference.

In other embodiments, the EC₅₀ of an inventive compound for the CB2 receptor is determined according to any one of the above CB2 receptor ligand binding assay methods. In another embodiment, the EC₅₀ is according to any assay method which studies binding at physiological pH or physiologically relevant conditions. In another embodiment, the EC₅₀ is determined according to any assay method which studies binding at physiological pH and ionic strength. Preferred assay incubation temperatures range from 20° C.-37° C. Temperatures may be lower or higher. For instance, incubation temperatures of just a few degrees or 0° C. may be useful in preventing or slowing the degradation of enzymatically unstable ligands. Inhibitors of FAAH may also be added to protect antagonists from degradation.

### PPARα Agonists for Use According to the Invention.

A preferred PPARα agonist is a fatty acid alkanolamide compound including, but not limited to OEA. Oleoylethanolamide (OEA) refers to a natural lipid of the following structure:

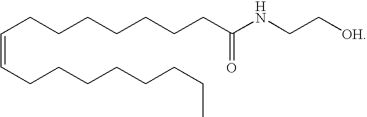

OEA-like compounds and OEA-like modulators for use as PPARα agonists according to the invention include, but are not limited to fatty acid ethanolamide compounds, and their homologues. A variety of OEA-like compounds and OEA-like modulators are contemplated. These compounds include compounds having the following general formula:

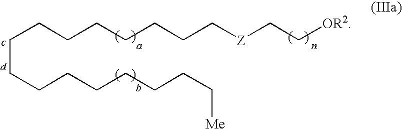

In this formula, n is any number from 0 to 5 and the sum of a and b can be any number from 0 to 4. Z is a member selected from —C(O)N(R^{o})—; —(R^{o})NC(O)—; —OC(O)—; —(O)CO—; O; NR^{o}; and S, in which R^{o} and R² are independently selected from the group consisting of alkyl, hydrogen, C₁-C₆ alkyl, (C₁-C₆) acyl, alkyl, and aryl. Up to eight hydrogen atoms of the compound may also be substituted by methyl group or a double bond. In addition, the molecular bond between carbons c and d may be unsaturated or saturated. In some embodiments, the fatty acid ethanolamide of the above formula is a naturally occurring compound. In some preferred embodiments, the alkyl substituents are each alkyl.

OEA-like compounds and OEA-like modulators of the invention also include compounds of the following formula:

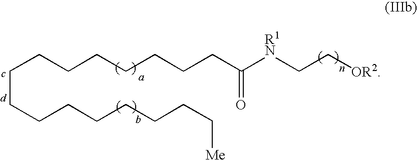

In one embodiment, the compounds of Formula IIIba have n from 0 to 5; and a sum of a and b that is from 0 to 4; and members R¹ and R² independently selected from the group consisting of hydrogen, C₁-C₆ alkyl, (C₂-C₆) acyl and aryl. In this embodiment, up to eight hydrogen atoms of the fatty acid portion and alkanolamine (e.g., ethanolamine) portion of compounds of the above formula may also be substituted by methyl or a double bond if adjacent carbons. In addition, the molecular bond between carbons c and d may be unsaturated or saturated. In some embodiments with acyl groups, the acyl groups may be the propionic, acetic, or butyric acids and attached via an ester linkage as R² or an amide linkage as R¹. In some embodiments, a H atom attached to a carbon atom of a compound of the above formula is replaced with a halogen atom, preferably a Cl atom or a F atom.

In addition, the PPARα agonists need not be an OEA-like compound (e.g., OEA, fatty acid amide or homolog thereof). In some embodiments, the OEA-like modulator is a compound such as taught in U.S. Pat. No. 6,200,998 (hereby incorporated by reference) that are PPARα A activators. This reference teaches PPAR agonist compounds of the general formula:

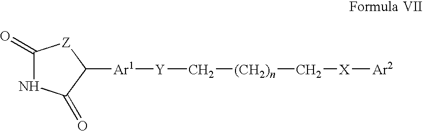

In the above formula, Ar¹ is (1) arylene or (2) heteroarylene, wherein arylene and heteroarylene are optionally substituted with from 1 to 4 groups selected from R^{a} (defined below); Ar² is (1) ortho-substituted aryl or (2) ortho-substituted heteroaryl, wherein said ortho substituent is selected from R (defined below); and aryl and heteroaryl are optionally further substituted with from 1-4 groups independently selected from R^{a}; X and Y are independently O, S, N—R^{b} (defined below), or CH₂; Z is O or S; n is 0 to 3; R is (1) C₃₋₁₀ alkyl optionally substituted with 1-4 groups selected from halo and C₃₋₆ cycloalkyl, (2) C₃₋₁₀ alkenyl, or (3) C₃₋₈ cycloalkyl; R^{a} is (1) C₁₋₁₅ alkanoyl, (2) C₁₋₁₅ alkyl, (3) C₂₋₁₅ alkenyl, (4) C₂₋₁₅ alkynyl, (5) halo, (6) OR^{b}, (7) aryl, or (8) heteroaryl, wherein said alkyl, alkenyl, alkynyl, and alkanoyl are optionally substituted with from 1-5 groups selected from R^{c} (defined below), and said aryl and heteroaryl optionally substituted with 1 to 5 groups selected from R^{d} (defined below); R^{b} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl, wherein alkyl, alkenyl, alkynyl are optionally substituted with one to four substituents independently selected from R^{c}, and cycloalkyl, aryl and heteroaryl are optionally substituted with one to four substituents independently selected from R^{d}; or R^{c} is (1) halo, (2) aryl, (3) heteroaryl, (4) CN, (5) NO₂, (6) OR^{f}; (7) S(O)ₘR^{f}, m=0, 1 or 2, provided that R^{f} (defined below) is not H when m is 1 or 2; (8) NR^{f}R^{f} (9) NR^{f}COR^{f}, (10) NR^{f}CO₂R^{f}, (11) NR^{f}CON(R^{f})₂, (12) NR^{f}SO₂R^{f}, provided that R^{f} is not H, (13) COR^{f}, (14) CO₂R^{f}, (15) CON(R^{f})₂, (16) SO₂N(R^{f})₂, (17) OCON(R^{f})₂, or (18) C₃₋₈ cycloalkyl, wherein said cycloalkyl, aryl and heteroaryl are optionally substituted with 1 to 3 groups of halo or C₁₋₆ alkyl; R^{d} is (1) a group selected from R^{c}, (2) C₁₋₁₀ alkyl, (3) C₂₋₁₀ alkenyl, (4) C₂₋₁₀ alkynyl, (5) aryl C₁₋₁₀ alkyl, or (6) heteroaryl C₁₋₁₀ alkyl, wherein alkyl, alkenyl, alkynyl, aryl, heteroaryl are optionally substituted with a group independently selected from R^{e}; R^{e} is (1) halogen, (2) amino, (3) carboxy, (4) C₁₋₄ alkyl, (5) C₁₋₄ alkoxy, (6) hydroxy, (7) aryl, (8) aryl C₁₋₄ alkyl, or (9) aryloxy; R^{f} is (1) hydrogen, (2) C₁₋₁₀ alkyl, (3) C₂₋₁₀ alkenyl, (4) C₂₋₁₀ alkynyl, (5) aryl, (6) heteroaryl, (7) aryl C₁₋₁₅ alkyl, (8) heteroaryl C₁₋₁₅ alkyl, (9) C₁₋₁₅ alkanoyl, (10) C₃₋₈ cycloalkyl; wherein alkyl, alkenyl, alkynyl, aryl, heteroaryl, alkanoyl and cycloalkyl are optionally substituted with one to four groups selected from R^{e}.

Also preferred are those PPARα specific activators as taught in U.S. Pat. No. 5,859,051. These activators have the following general formula as set forth in the U.S. Pat. No. 5,589,051:

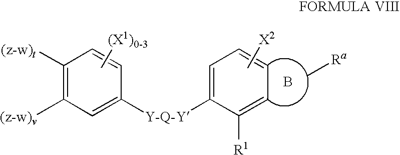

In the embodiments according to Formula VIII, R¹ is selected from a group consisting of: H, C₁₋₁₅ alkyl, C₂₋₁₅ alkenyl, C₂₋₁₅ alkynyl and C₃₋₁₀ cycloalkyl, said alkyl, alkenyl, alkynyl, and cycloalkyl optionally substituted with 1 to 3 groups of R^{a} (defined below); R³ is selected from a group consisting of: H, NHR¹, NHacyl, C₁₋₁₅ alkyl, C₃₋₁₀ cycloalkyl, C₂₋₁₅ alkenyl, C₁₋₁₅ alkoxy, CO₂ alkyl, OH, C₂₋₁₅ alkynyl, C₅₋₁₀ aryl, C₅₋₁₀ heteroaryl said alkyl, cycloalkyl, alkenyl, alkynyl, aryl and heteroaryl optionally substituted with 1 to 3 groups of R^{a}; (Z-W—) is Z-CR⁶R⁷—, Z-CH.═CH—, or:

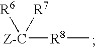

R⁸ is selected from the group consisting of CR⁶R⁷, O, NR⁶, and S(O)_{P}; R⁶ and R⁷ are independently selected from the group consisting of H, C₁₋₆ alkyl; B is selected from the group consisting of: 1) a 5 or 6 membered heterocycle containing 0 to 2 double bonds, and 1 heteroatom selected from the group consisting of O, S and N, the heteroatom being substituted at any position on the five or six membered heterocycle, the heterocycle being optionally unsubstituted or substituted with 1 to 3 groups of R^{a}; 2) a 5 or 6 membered carbocycle containing 0 to 2 double bonds, the carbocycle optionally unsubstituted or substituted with 1 to 3 groups of R^{a} at any position on the five or six membered carbocycle; and 3) a 5 or 6 membered heterocycle containing 0 to 2 double bonds, and 3 heteroatoms selected from the group consisting of O, N, and S, which are substituted at any position on the five or six membered heterocycle, the heterocycle being optionally unsubstituted or substituted with 1 to 3 groups of R^{a}; X¹ and X² are independently selected from a group consisting of: H, OH, C₁₋₁₅ alkyl, C₂₋₁₅ alkenyl, C₂₋₁₅ alkynyl, halo, OR³, ORCF₃, C₅₋₁₀ aryl, C₅₋₁₀ aralkyl, C₅₋₁₀ heteroaryl and C₁₋₁₀ acyl, said alkyl, alkenyl, alkynyl, aryl and heteroaryl optionally substituted with 1 to 3 groups of R^{a}; R^{a} represents a member selected from the group consisting of: halo, acyl, aryl, heteroaryl, CF₃, OCF₃, —O—, CN, NO₂, R³, OR³; SR³, ═N(OR), S(O)R³, SO₂R³, NR³R³, NR³COR³, NR³CO₂R³, NR³CON(R³)₂, NR³SO₂R³, COR³, CO₂R³, CON(R³)₂, SO₂N(R³)₂, OCON(R³)₂ said aryl and heteroaryl optionally substituted with 1 to 3 groups of halo or C₁₋₆ alkyl; Y is selected from the group consisting of: S(O)ₚ, —CH₂—, —C(O)—, —C(O)NH—, —NR—, —O—, —SO₂NH—, —NHSO₂; Y¹ is selected from the group consisting of: O and C; Z is selected from the group consisting of: CO₂R³, R³CO₂R³, CONHSO₂Me, CONHSO₂, CONH₂ and 5-(1H-tetrazole); t and v are independently 0 or 1 such that t+v=1 Q is a saturated or unsaturated straight chain hydrocarbon containing 2-4 carbon atoms and p is 0-2 with the proviso when Z is CO₂R³ and B is a 5 membered heterocycle consisting of O, R³ does not represent methyl.

Additional compounds suitable for practicing the inventive methods include compounds taught in U.S. Pat. No. 5,847,008, U.S. Pat. No. 6,090,836 and U.S. Pat. No. 6,090,839, U.S. Pat. No. 6,160,000 each of which is herein incorporated by reference in its entirety to the extent not inconsistent with the present disclosure.

Additionally a variety of suitable PPAR agonists and activators for screening are taught in U.S. Pat. No. 6,274,608. Aryl and heteroaryl acetic acid and oxyacetic acid compounds are taught for instance in U.S. Pat. No. 6,160,000; substituted 5-aryl-2,4-thiazolidinediones are taught in U.S. Pat. No. 6,200,998; other compounds including PPARα-specific polyunsaturated fatty acids and eicosanoids are known as described in Forman, B M, Chen, J, and Evans R M, PNAS 94:4312-4317 and PCT Patent Publication No. WO 97/36579, published Oct. 9, 1997). The compositions of these publications, which are each herein incorporated by reference in their entirety to the extent not inconsistent with the present disclosure can be screened by the methods provide below to provide the PPARα specific agonists of the invention which are useful in treating neuropathic pain.

In some embodiments, the PPARα agonist is clofibrate or a derivative of clofibrate. Such compounds include, but are not limited to, clofibrate (i.e., 2-(4-chlorophenoxy)-2-methylpropanoic acid, ethyl ester); fenofibrate, (1-methylethyl 2-[4-(4-chlorobenzoyl)phenoxy]-2-methylpropanoate; 2-[4-(4-chlorobenzoyl)phenoxy]-2-methylpropanoic acid, 1-methylethyl ester); bezafibrate (2-[4-[2-[(4-chlorobenzoyl)amino]-ethyl]phenoxy]-2-methyl-propanoic acid, gemfibrozil: 5-(2,5-dimethylphenoxy)-2,2-dimethylpentanoic acid and ciprofibrate.

Other PPARα agonists suitable for use in the methods and compositions of the invention are clofibrate derivative compounds of the following formula or their pharmaceutically acceptable salts:

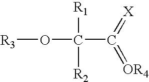

wherein R₁ and R₂ may be the same or different and are each a hydrogen atom or a substituted or unsubstituted alkyl, alkoxy, or phenoxy group, R₃ is a substituted or unsubstituted aryl group phenyl group and X is hydrogen (2H) or oxygen, and R₄ is H or alkyl. In one embodiment, the R₃ aryl group is substituted or unsubstituted phenyl, preferably monosubstituted. In another embodiment, X is O and R₃ is a mono-, di- or tri-substituted phenyl group, bearing one, two or three identical or different substituents for an aryl group and R₁ and R₂ are each, independently, a hydrogen atom or an alkyl group. In a further embodiment, R₃ is a is a mono-, di- or tri-substituted phenyl group, bearing one, two or three identical or different substituents which are one or more of the following, namely halogen atoms and alkyl, alkoxy, aryl, heteroaryl, or hydroxy groups, and R₁ and R₂ are each, independently, a hydrogen atom or an alkyl group, and R₄ is H or alkyl.

Additional PPARα agonists for use according to the invention include:

WY-14,643 (i.e., [4-chloro-6-(2,3-xylidino)-2-pyrimidinylthio]acetic acid)

Fenofibrate (see U.S. Pat. Nos. 5,830,148; 6,074,670; 5,827,536; 5,545,628; 6,277,405 and Casas, F. et al., FEBS Lett., 482(1-2): 71-4 (2000)).

Medium and long chain fatty acids (see U.S. Pat. Nos. 6,008,237; 6,200,998)

Aryithiazolidinedione derivatives (see U.S. Pat. Nos. 6,200,998; 6,008,23)

Propionic acid derivatives (see U.S. Pat. No. 6,306,854)

Pioglitazone (see Smith, U., Int. J. Clin. Pract. Suppl., 121: 13-18 (2001))

Benzafibrate(bezafibrate) (see Yoshikawa et al., Eur. J. Pharmacol., 426(3): 2001-6 (2001); Bonilla, S. et al., J. Physiol. Biochem., 57(1): 1-8 (2001); Pedraza, N., et al., Diabetes, 49(7): 1224-30 (2000)0

(−) DRF2725 (i.e., (−)3-[4-[2-(phenoxazin 10-yl)ethoxyl]phenyl]-2-ethoxypropionic acid) (see Lohray, B. B. et al., J. Med. Chem., 44(15): 2675-8 (2001)

BM-17.0744 (see Carroll, R. et al., Physiol. Heart Circ. Physiol., 281(2): H888-94 (2001)) ciprofibrate (see Latruffe, N. et al., Cell Biochem. Biophys., 32 Spring: 213-20 (2000));

Omega-3-fatty acids, including docosahexanoic acid (see Diep, Q. H. et al., Hypertension, 36(5): 851-5 (2000))

Clofibrate (see Mehendale, H. M., Toxicol. Sd., 57(2): 187-90 (2000))

JTT-501 4-[4-[2-(5-methyl-2-phenyl]-4-oxazolyl)ethoxy)benzyl]-3,5-isoxazolidiedione (see Shibata, T. et al., Br. J. Pharmacol., 30(3): 495-504 (2000)

Trichloroacetate, dichloroacetate; DHEA-S dehydroepiandrosterone-3-beta-sulfate (see—Zhou, Y. C. et al, Environ. Health Perspect., 106(Suppl.; 4): 983-988 (1998)

Unsaturated C: 18 fatty acids (e.g., arachidonic acid, leukotriene B4) (see Lin, Q., et al., Biochemistry, 38(1): 185-90 (1999)

Perfluorooctanoic acid

Fatty aryls (e.g., 4-iodophenylbutyrate, 4-chlorophenylbutyrate; clofibate; phenylbutyrate; naphthylacetate; 2,4-D; 4-chlorophenylacetate; phenylacetate; indoacetate) (see Pineau, T. et al., Biochem. Pharmacol., 53(4): 659-67 (1996);

Fibrates (e.g., beclobrate; bezafibrate; ciprofibrate; clofibrate; clofibride; etofibrate; fenofibrate; gemfibrozil; simfibrate) (see Staels, B. et al, Biochimie, 79(2-3): 95-9 (1997)).

U.S. Pat. No. 6,306,854 describes compounds that can serve as the PPARα agonists for use according to the present invention. The compounds have the general structure of formula XI, or a salt thereof, where the general structure is:

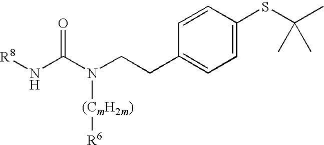

wherein m is from 0 to 20, R⁶ is selected from the group consisting of hydrogen and

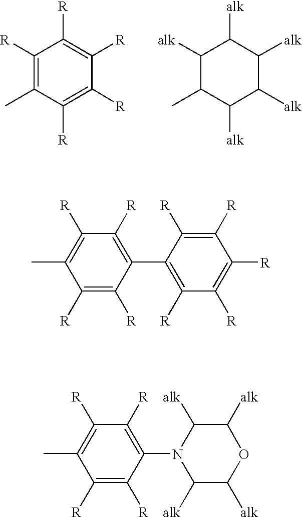

and R⁸ is selected from the group consisting of

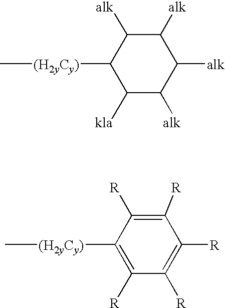

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and. which can contain hetero atoms such as nitrogen, oxygen, or sulfur and which can contain functional groups such as ketone or ester, cycloalkyl containing 3 try 7 carbon atoms, or two R groups bonded to adjacent carbon atoms can, together with the carbon atoms to which they are bonded, form an aliphatic or aromatic ring or mufti ring system, and where each depicted ring has no more that 3 alk groups.

Examples of preferred compounds that have the structure of the above formula include:
2-(4-(2-(1-(4-biphenylethyl)-3-cyclohexylureido)ethyl)phenylthio)-2-methylpropionic acid,
2-(4-(2-(1-(2-(4-morpholinophenyl)ethyl-3-cyclohexylureido)ethyl)phenylthio)-2-methylpropionic acid;
2-(4-(2-(1-(cyclohexanebutyl)-3-cyclohexylureido)ethyl)phenylthio)-2-methylpropionic acid;
2-(4-(2-(1-heptyl-3-(2,4-difluorophenyl)ureido)ethyl)phenylthio)-2-methylpropionic acid,
2-(4-(2-(1-(2-chloro-4-(2-trifluoromethylphenyl)phenylmethyl)-3-(cyclohexyl)ureido)ethyl)phenylthio)-2-methylpropionic acid, or a salt thereof.

In some embodiments, the PPARα agonist is selected from the following list:

WY-14,643, Fibrates (Most if not all are fibrates)
Clofibrate
Clofibride
Fenofibrate
clorofibrate
Benzafibrate
Ciprofibrate
Beclofibrate
(beclobrate)
etofibrate
simfibrate
gemfibrozil
Nafenopin
Benfluorex

pioglitazone

(−) DRF2725

BM-17.0744

docosahexanoic acid

JTT-501

Trichloroacetate

Dichloroacetate

DHEA

DHEA-S

leukotriene B4

Fatty acids and their derivatives which activate PPARα

Fatty acid ethanolamides and their derivatives which activate PPARα

ETYA

GW 9578

GW 7647

GW 2331

GW 9578

Tetradecylthioacetic acid (TTA)

8(S)-HETE
BRL 49653

Each of the above patents cited in this section are incorporated by reference herein with particular reference to the PPARα agonist compounds and compositions they disclose.

### Identification of PPARα Agonists

While many PPARα Agonist compounds are known in the art, identification and characterization of suitable novel compounds that specifically or selectively bind PPARα can be accomplished by any means known in the art, such as, for example, electrophoretic mobility shift assays and competitive binding assays. Preferably PPARα specific binding compounds have at least 5-10 fold, preferably 10-100 fold, more preferably 100-500 fold, most preferably greater than 1000 fold specificity for PPARα compared to other PPAR subtypes. Mammalian PPAR subtypes (e.g., rat, mouse, hamster, rabbit, primate, guinea pig) are preferably used. More preferably, human PPAR subtypes are used.

Electrophoretic Mobility Shift Assays

Electrophoretic mobility shift assays can be used to determine whether test compounds bind to PPARα and affect its electrophoretic mobility. (Forman, et al. (1997) PNAS 94:4312 and Kliewer, et al. (1994) PNAS 91:7355). Electrophoretic mobility shift assays involve incubating a PPAR-RXR with a test compound in the presence of a labeled nucleotide sequence. Labels are known to those of skill in the art and include, for example, isotopes such as, ³H, ¹⁴C, ³⁵S, and ³²P, and non-radioactive labels such as fluorescent labels or chemiluminescent labels. Fluorescent molecules which can be used to label nucleic acid molecules include, for example, fluorescein isothiocyanate and pentafluorophenyl esters. Fluorescent labels and chemical methods of DNA and RNA fluorescent labeling have been reviewed recently (Proudnikov et al., 1996, Nucleic Acids Res. 24:4535-42).

Chemiluminescent labels and chemiluminescent methods of labeling DNA and RNA have been reviewed recently (Rihn et al., 1995, J. Biochem. Biophys. Methods 30:91-102). Use of non-radioactive labeled probes directly for studying protein-polynucleotide interactions with EMSA has been described. (U.S. Pat. No. 5,900,358). The mixtures can be separated, run on a separate lane of a gel, and autoradiographed. For example, if a test compound does not result in a change in the bands seen in the control lane then the test compound is not a candidate PPARα specific binding compound. On the other hand, if a change in intensity in at least one of the bands is seen, then the compound is a candidate PPARα specific binding compound. (U.S. Pat. No. 6,265,160). The incubation mixture is then electrophoretically separated and the resulting gel exposed to X-ray film. The resulting autoradiograph may have one or more bands representing slowly migrating DNA-protein complexes. This control lane can indicate the mobility of the complex between the DNA probe and PPAR.

Monoclonal antibodies specific for PPAR subtypes can be used to identify PPARα specific binding compounds in modified electrophoretic mobility shift assays. Purified PPARβ, PPARα or PPARγ can be incubated with an appropriate amount of a test compound in the presence of RXR. For these assays, the test compound need not be labeled. PPAR subtype specific monoclonal antibodies can be incubated with the PPAR-RXR-test compound mixture. For instance, test compounds that bind PPAR induce supershifting of the PPAR-RXR complex on a gel (Forman, et al. (1997), PNAS 94:4312) which can be detected by anti-PPAR monoclonal antibodies using a Western blot (immunoblot).

Generation of monoclonal antibodies has been previously described and can be accomplished by any means known in the art. (Buhring et al. in Hybridoma 1991, Vol. 10, No. 1, pp. 77-78). For example, an animal such as a guinea pig or rat, preferably a mouse is immunized with a purified PPAR subtype, the antibody-producing cells, preferably splenic lymphocytes, are collected and fused to a stable, immortalized cell line, preferably a myeloma cell line, to produce hybridoma cells which are then isolated and cloned. (U.S. Pat. No. 6,156,882).

Western blots generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind PPAR subtypes. These antibodies may be directly labeled or alternatively may be subsequently detected using labeled antibodies (e.g., labeled sheep anti-mouse antibodies) that specifically bind to the anti-PPAR antibodies.

The particular label or detectable group used in the assay is not a critical aspect of the invention, as long as it does not significantly interfere with the specific binding of the PPAR subtype specific ligand used in the assay. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), and colorimetric labels such as colloidal gold or colored glass or plastic beads (e.g., polystyrene, polypropylene, latex, etc.).

The molecules can be conjugated directly to signal generating compounds, e.g., by conjugation with an enzyme or fluorophore. Enzymes of interest as labels will primarily be hydrolases, particularly phosphatases, esterases and glycosidases, or oxidases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple calorimetric labels may be detected simply by observing the color associated with the label. Thus, in various dipstick assays, conjugated gold often appears pink, while various conjugated beads appear the color of the bead. In particular, one of ordinary skill in the art would appreciate that fluorescence resonance energy transfer (FRET) can be used.

Other assay formats include liposome immunoassays (LIA), which use liposomes designed to bind specific molecules (e.g., antibodies) and release encapsulated reagents or markers. The released chemicals can be then detected according to standard techniques (see Monroe et al., Amer. Clin. Prod. Rev. 5:34-41 (1986)).

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Competitive Binding Assays

In addition to electrophoretic mobility shift assays, competitive binding assays can be used to identify PPARα specific binding compounds. In competitive assays, the binding of test compounds to PPARα can be determined by measuring the amount of OEA that they displaced (competed away) from PPARα. Purified PPARβ, PPARα, and PPARγ receptors can be incubated with varying amounts of a test compound in the presence of labeled ligands specific for each PPAR subtype. For example, GW 2433 and L-783483 can be used in conjunction with PPAR□; GW 2331 or OEA can be used in conjunction with PPAR□□ and rosiglitazone, AD-5075, and SB-236636 can be used in conjunction with PPAR□. Specificity of the test compound for each PPAR subtype can be determined by detection of the amount of labeled ligand that remains bound to each PPAR after incubation with the test compound. Labels are discussed above.

### Measuring Activation of PPARα

The ability of an OEA-like compound or OEA-like modulator to activate PPARα can be measured using any means known in the art. PPARα activators act by inducing PPARα-RXR heterodimer formation. The PPARα-RXR heterodimer then binds to DNA sequences containing AGGTCAnAGGTCA and activates PPAR target genes. Preferably PPARα activators activate PPARα by at least 5-10 fold, more preferably 10-100 fold, more preferably 100-500 fold, more preferably 500-100 fold, most preferably greater than 1000 fold above base level. PPARα can be transfected into cells. The transfected cells can be then exposed to candidate compounds. Any means known in the art can be used to determine whether PPARα is activated by the candidate compound, such as for example, by measuring levels of reporter gene expression and cell proliferation.

Transfection of PPAR into Cells

Any of the well-known procedures for introducing foreign nucleotide sequences into host cells may be used to transfect PPARα into cells such as, for example, calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). Methods of transfection have also been described in U.S. Pat. Nos. 5,616,745, 5,792,6512, 5,965,404, and 6,051,429 and in Current Protocols in Molecular Biology, Ausubel, et al., ed. (2001). It is only necessary that the particular genetic engineering procedure used be capable of successfully introducing at least one gene into the host cell capable of expressing PPARα. After the expression vector is introduced into the cells, the transfected cells can be cultured under conditions favoring expression of PPARα.

Detection of Reporter Gene Expression

Expression of reporter genes in response to compounds identified as binders of PPARα may also be used to measure PPARα activation. PPARα may be co-transfected with reporter genes known in the art such as, for example, luciferase, β-galactosidase, alkaline phosphatase, fluorescent green protein, or chloramphenicol acetyltransferase. The transfected cells can be exposed to appropriate concentrations of candidate compounds with OEA as a positive control. Reporter gene expression will be induced by compounds that bind and activate PPARα. Thus, compounds that induce reporter gene expression can be identified as activators of PPARα. (Forman, et al. (1997) PNAS 94:4312). Preferably the compounds induce reporter gene expression at levels at least 5-10 fold, more preferably 10-100 fold, more preferably 100-500 fold, more preferably 500-1000 fold, most preferably greater than 1000 fold greater than the negative control.

Proliferation of PPARα Transfected Cells

PPARα activation may also be measured by proliferation of cells transfected with PPARα. Cell proliferation can be induced by compounds that bind and activate PPARα, such as, for example, OEA. Thus, PPARα transfected cells can be exposed to appropriate concentrations of candidate compounds with OEA as a positive control. Compounds that induce cells to proliferate can thereby be identified as activators of PPARα. Cell proliferation can be measured, for example, by incorporation of 5′-bromo-2′deoxyuridine or 3H-thymidine as described in Jehl-Pietri, et al., (2000) Biochem J. 350:93 and Zoschke and Messner (1984) Clin. Immunol. Immunopath. 32:29, respectively. Preferably the compounds induce cell proliferation at levels at least 5-10 fold, more preferably 10-100 fold, more preferably 100-500 fold, more preferably 500-1000 fold, most preferably greater than 1000 fold greater than the negative control.

### Methods for Assessing Ability of a Compound to Modulate Stress-Induced Responses Stress-Induced Analgesia or Pain-Relief

Methods for screening FAAH inhibitors for an antinociceptive effect are well known to one of ordinary in the art. For instance, the test compounds can be administered to the subject animals in the mouse hot-plate test (Beltramo et al., Science, 277:1094-1097 (1997)) and the mouse formalin test and the nociceptive reactions to thermal or chemical tissue damage measured. See also U.S. Pat. No. 6,326,156 which teaches methods of screening for antinociceptive activity. See Cravatt et al. Proc. Natl. Acad. Sci. U.S.A. 98:9371-9376 (2001). A method of testing for antinociception is set forth in the Examples.

A fully automatic tail-flick analgesiameter (IITC Model 336; Woodland Hills, Calif.) may be used to assess tail-flick latencies. This assessment of tail-flick latency is not subject to bias. Removal of the tail from the radiant heat source is initiated by the rat, which automatically terminates the heat stimulus. The tail-flick latency is calculated by the electronic analgesia meter without intervention of the experimenter. Tail-flick latencies can be assessed in a manner identical to that described in the art (Walker et al. PNAS 96, 12198-12203, 1999; Martin et al. J Nsci 16, 6601-6611, 1996).

The diagnosis and assessment of neuropathic pain is well known to one of ordinary skill in the art. Pain can be identified and assessed according to its onset and duration, location and distribution, quality and intensity, and secondary signs and symptoms (e.g., mood, emotional distress, physical or social functioning), and triggering stimulus or lack thereof. For human subject, often subjective pain assessment scales are used to measure intensity. Such scales may grade pain intensity verbally ranging from no pain-mild pain-moderate pain-severe pain-very severe pain and worst possible pain, or on a numeric scale from 1 (no pain) to 5 (moderate pain) to 10 (worst possible pain).

Suitable animal models for testing the ability of agents to treat neuropathic pain are also known to one of ordinary skill in the art. Such methods have been the subject of recent review (Wang et al. Advanced Drug Delivery Reviews 55:949 (2003)) which is incorporated by reference herein in its entirety. Methods of assessing neuropathic pain include 1) the weight drop or contusion model of Allen; 2) the photochemical SCI model: 3) the excitotoxic spinal cord injury model; 4) the neuroma model; 5) the chronic constriction injury model of Bennett; 6) the partial sciatic nerve ligation model; 7) the L5/L6 spinal ligation model; 8) the sciatic cryoneurolysis model; and 9) the sciatic inflammatory neuritis model. In addition there are a variety of models for studying the neuropathic pain of diabetes polyneuropathy; toxic neuropathies; and various bone cancer models.

### Screening for Anxiolytic Activity

One of ordinary skill in the art would appreciate that there are a number of animal models available for assessing the antianxiety effects of a compound. Two pharmacologically validated animal models of anxiety are the elevated zero maze test, and the isolation-induced ultrasonic emission test. The zero maze consists of an elevated annular platform with two open and two closed quadrants and is based on the conflict between an animal's instinct to explore its environment and its fear of open spaces, where it may be attacked by predators (Bickerdike, M. J. et al., Eur. J. Pharmacol., 271, 403-411 (1994); Shepherd, J. K. et al., Psychopharmacology, 116, 56-64 (1994)). Clinically used anxiolytic drugs, such as the benzodiazepines, increase the proportion of time spent in, and the number of entries made into, the open compartments.

A second test for an antianxiety compound is the ultrasonic vocalization emission model, which measures the number of stress-induced vocalizations emitted by rat pups removed from their nest (Insel, T. R. et al., Pharmacol. Biochem. Behav., 24, 1263-1267 (1986); Miczek, K. A. et al., Psychopharmacology, 121, 38-56 (1995); Winslow, J. T. et al., Biol. Psychiatry, 15, 745-757 (1991).

A large number of animal models have been developed in the attempt to predict the anxiolytic activity of novel compounds in man. Many of these paradigms evaluate animal behavior in a so-called “conflict” situation, i.e. a behavioral response is simultaneously under the influence of two opposing motivational states such as approach and avoidance tendencies. Probably the best known model is the conditioned punishment conflict paradigm in which animals are trained to voluntarily exhibit a certain response (e.g. pressing a lever) in order to receive a reward (e.g. food for a hungry animal). Once the animals exhibit a constant rate of lever-press responding, then short periods are introduced (usually signaled by visual or acoustic signals) during which lever pressing is simultaneously rewarded by food and punished by mild electrical foot shock. Animals exhibit a markedly reduced response rate during these conflict periods, which are also characterized by various overt signs of emotionality. The characteristic effect of benzodiazepine receptor agonists, for example the anxiolytic diazepam, is the disinhibition of punished behavior (resulting in an increase in the rate of responding under punishment) at doses that fail to disrupt unpunished responding. Furthermore, these same active drugs produce an anxiolytic-like effect in the absence of actual punishment, i.e. when the rate of lever pressing is reduced by conditioned fear of punishment. The conflict task does not require conditioned behavioral responses: naive thirsty animals can be offered the opportunity to drink, with drinking punished via contact with an electrified spout. Such punishment-suppressed drinking is disinhibited dose-dependently by benzodiazepine receptor agonists (e.g., diazepam). Exploratory activity can likewise be decreased by contingent punishment and released by treatment with known anxiolytics. Conflict models without punishment are based on the presence of the natural opposing motivational states, on the one hand the tendency to explore and, on the other hand, fear of a novel environment (e.g. dark-light chamber task, elevated plus-maze, consumption of unfamiliar food or normal food in an unfamiliar environment, social interaction between animals unfamiliar with each other). While it is obvious to ascribe the behavioral disinhibitory effect of benzodiazepine receptor agonism in these experimental situations to an anxiolytic-like action, their effect can also be interpreted as a general reduction of the influence of aversive factors or even to an impaired ability to withhold innate or conditioned responses. An anti-frustration effect resulting from benzodiazepine receptor agonism is suggested by the increase of responding which is maintained by response-contingent reward in the situation in which the reward is reduced or omitted. Electrical stimulation of the periaqueductal gray area of the midbrain via chronically implanted electrodes in animals is aversive and elicits a number of emotional reactions; benzodiazepine receptor agonists increase the aversive threshold. States of acute anxiety characterised by behavioral and physiological symptoms (cardiovascular, endocrine) can be induced by chemicals known to be anxiogenic in man, e.g. convulsants such as pentylenetetrazol, inverse agonists at the benzodiazepine receptor agonists administered in subconvulsive doses, or even abrupt drug withdrawal after chronic treatment with high doses of sedatives. Ultrasonic distress cries by rat pups acutely separated from their mothers are decreased by benzodiazepine receptor agonists.

### Screening for Antidepressant Activity

Animal models for depression are also well known to those of ordinary skill in the art. For instance, the effect of the compound of the invention in the treatment of depression can be tested in the model of chronic mild stress induced anhedonia in rats. This model is based on the observation that chronic mild stress causes a gradual decrease in sensitivity to rewards, for example consumption of sucrose, and that this decrease is dose-dependently reversed by chronic treatment with antidepressants. The method has previously been described and more information with respect to the test appears from Willner, Paul, Psychopharmacology, 1997, 134, 319-329.

Another test for antidepressant activity is the forced swimming test (Nature 266, 730-732, 1977) In this test, animals are administered an agent preferably by the intraperitoneal route or by the oral route 30 or 60 minutes before the test. The animals are placed in a crystallizing dish filled with water and the time during which they remain immobile is clocked. The immobility time is then compared with that of the control group treated with distilled water. Imipramine 25 mg/kg. can be used as the positive control. The antidepressant compounds decrease the immobility time of the mice thus immersed.

Another test for antidepressant activity is the caudal suspension test on the mouse (Psychopharmacology, 85, 367-370, 1985) In this test, animals are preferably treated with the study compound by the intraperitoneal route or by the oral route 30 or 60 minutes before the test. The animals are then suspended by the tail and their immobility time is automatically recorded by a computer system. The immobility times are then compared with those of a control group treated with distilled water. Imipramine 25 mg/kg can be used as the positive control. Antidepressant compounds decrease the immobility time of the mice.

Another test for screening antidepressants is the DRL-72 TEST. This test, carried out according to the protocol of Andrews et al [“Effects of imipramine and mirtazapine on operant performance in rats”—Drug Development Research 32, 58-66 (1994)], gives an indication of antidepressant-like activity. See also U.S. Pat. No. 6,403,573.

Additional animal models for screening are well known to one of ordinary skill in the art. For instance, see U.S. Pat. No. 5,952,315.

### Pharmaceutical Compositions.

Another aspect of the present invention provides pharmaceutical compositions which comprises a MGL inhibitor and, optionally, one or more additional anti-nociceptive or analgesic agents (e.g., an opioid, FAAH inhibitor, anandamide transport inhibitor, PPARα agonist, COX-2 inhibitor, or NSAID). The composition can further comprise a pharmaceutically acceptable carrier.

The compositions can be suitable for oral, rectal, topical, parenteral (including subcutaneous, intramuscular, and intravenous), ocular (ophthalmic), pulmonary (nasal or buccal inhalation), or nasal administration, although the most suitable route in any given case will depend in part on the nature and severity of the conditions being treated and on the nature of the active ingredient. An exemplary route of administration is the oral route. The compositions may be conveniently presented in unit dosage form and prepared by any of the methods well-known in the art of pharmacy.

In practical use, the active agents for use according to the invention (e.g., MGL inhibitor, FAAH inhibitors,) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers can be employed. If desired, tablets may be coated by standard aqueous or nonaqueous techniques. Such compositions and preparations can contain at least 0.1 percent of active compounds. The percentage of active compound in these compositions may, of course, be varied and may conveniently be between about 2 percent to about 60 percent of the weight of the unit. The amount of active compound in such therapeutically useful compositions is such that a therapeutically effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. To prevent breakdown during transit through the upper portion of the GI tract, the composition may be an enteric coated formulation.

### Methods of Treatment

### Anxiety and Anxiety Related Disorders.

In some embodiments, the compounds, pharmaceutical compositions, and methods of treatment according to the invention are useful in treating anxiety and anxiety disorders or conditions. One of ordinary skill in the art is readily able to diagnose such conditions. The compounds and compositions are useful, for example in treating anxiety, clinical anxiety, panic disorder, agoraphobia, generalized anxiety disorder, specific phobia, social phobia, obsessive-compulsive disorder, acute stress disorder, and post-traumatic stress disorder; and adjustment disorders with anxious features, anxiety disorders due to general medical conditions, substance-induced anxiety disorders, and the residual category of anxiety disorder not otherwise specified. The treatment may be prophylactic or therapeutic. The treatment may be administered to a human subject. The compounds, compositions and methods may be used in otherwise healthy individuals who are not otherwise in need of any pharmaceutical intervention for a disease or condition such as insomnia or for pain relief.

In some embodiments, the compounds methods, and compositions of the invention may also be administered to treat anxiety in mammals, including cats, dogs, and humans. In some embodiments, the compounds may be used in otherwise healthy individuals who are not in need of pharmaceutical interventions for any other disease or disorder than anxiety or an anxiety disorder.

The compounds and compositions of the invention may be administered solely for the purposes of reducing the severity or frequency of anxiety or an anxiety disorder.

### Depression and Depressive Disorders

In some embodiments, the compounds, pharmaceutical compositions and method for treatment according to the invention are are useful in treating depression and depressive disorders or conditions. One of ordinary skill in the art is readily able to diagnose such conditions and disorders. The compounds and compositions are useful, for example in treating major depressive disorders (unipolar depression), dysthymic disorders (chronic, mild depression), and bipolar disorders (manic-depression). The depression may be clinical or subclinical depression. The treatment may be prophylactic or therapeutic. The treatment may be administered to a human subject.

In some embodiments, the compounds methods, and compositions of the invention may also be administered to treat depression in mammals, including cats, dogs, and humans. In some embodiments, the compounds may be used in otherwise healthy individuals who are not in need of pharmaceutical interventions for any other disease or disorder than depression or a depressive disorder.

The compounds and compositions of the invention may be administered solely for the purposes of reducing the severity or frequency of depression or a depressive disorder.

### Pain

As pain is a stressor itself, in some embodiments, the compounds, compositions, and methods of treatment according to the invention are administered to alleviate pain in a subject. One or ordinary skill in the art can identify severe pain conditions or stressful conditions likely to induce stress-induce analgesia. The treatment may be prophylactic or therapeutic. The treatment may be administered to a human subject in need of pain relief or modulation of stress-induced analgesia. The compounds and compositions of the invention may be administered solely for the purposes of reducing the severity or frequency or extent of pain. The treatment may be administered in a combination therapy with another pain reliever or an antiinflammatory agent.

Pain, in particular, can be a stressor, and also a condition subject to treatment according to the invention. Thus, in one aspect the invention is drawn to methods of treating chronic pain conditions, including neuropathic pain, and chronic or intermittent pain associated with chronic health conditions as such conditions are often substantial stressors. In other embodiments, the pain can be a neuropathic pain.

### Administration

The pharmaceutical compositions of the invention may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

The active agents are administered in therapeutically effective amounts. The exact dosage will depend upon the agent, mode of administration, on the therapy desired, form in which administered, the severity and condition of the subject to be treated and the body weight of the subject to be treated, and the preference and experience of the physician or veterinarian in charge. The active agents may, for instance, be effective over a wide dosage range. For example, in the treatment of adult humans, dosages from about 1 to about 1000 mg, about 100 to about 500 mg or about 1 to about 100 mg of may be needed. Doses of the 0.05 to about 100 mg, and more preferably from about 0.1 to about 100 mg, per day may be used. A most preferable dosage is about 0.1 mg to about 70 mg per day. In choosing a regimen for patients, it may frequently be necessary to begin with a dosage of from about 2 to about 1000 mg per day and to adjust the dosage periodically as needed to bring the condition under control or find a still reasonably effective, lower dosage. For example, in the treatment of adult humans, dosages from about 0.05 to about 100 mg, preferably from about 0.1 to about 100 mg, per day may be used.

Generally, active agents can be dispensed in unit dosage form comprising preferably from about 1.0 to about 1000 mg of active ingredient together with a pharmaceutically acceptable carrier per unit dosage. Usually, dosage forms suitable for oral, nasal, pulmonary or transdermal administration comprise from about 0.001 mg to about 1000 mg, preferably from about 0.1 mg to about 100 mg of the compounds admixed with a pharmaceutically acceptable carrier or diluent. For storage and use, these preparations preferably contain a preservative to prevent the growth of microorganisms.

Administration of an appropriate amount of the compounds may be by any means known in the art such as, for example, oral or rectal, parenteral, intraperitoneal, intravenous, subcutaneous, subdermal, intranasal, or intramuscular. In some embodiments, administration is transdermal. In yet other embodiments, administration is topical. An appropriate amount or dose of the candidate compound may be determined empirically as is known in the art. For example, with respect to neuropathic pain, depression or anxiety, a therapeutically effective amount is an amount sufficient to reduce the severity of pain, anxiety, or depression, respectively, as measured by subjective or objective indicia in the subject over time. The candidate compound can be administered as often as required to reduce or control pain, anxiety or depression, for example, hourly, every two, three, four, six, eight, twelve, or eighteen hours, daily in the case of chronic pain, or according to the actual or subjective perception of pain, anxiety or depression so as to reduce it to a more tolerable level, or in advance of activities likely to exacerbate the pain, anxiety or depression.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Injection solutions and suspensions can be prepared from sterile powders, granules, and tablets of the kind previously described. Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

With respect to transdermal routes of administration, methods for transdermal administration of drugs are disclosed in Remington's Pharmaceutical Sciences, 17th Edition, (Gennaro et al. Eds., Mack Publishing Co., 1985). Dermal or skin patches are a preferred means for transdermal delivery of the compounds of the invention. Patches preferably provide an absorption enhancer such as DMSO to increase the absorption of the compounds. Other methods for transdermal drug delivery are disclosed in U.S. Pat. Nos. 5,962,012, 6,261,595, and 6,261,595. Each of which is incorporated by reference in its entirety.

Preferred patches include those that control the rate of drug delivery to the skin. Patches may provide a variety of dosing systems including a reservoir system or a monolithic system, respectively. The reservoir design may, for example, have four layers: the adhesive layer that directly contacts the skin, the control membrane, which controls the diffusion of drug molecules, the reservoir of drug molecules, and a water-resistant backing. Such a design delivers uniform amounts of the drug over a specified time period, the rate of delivery has to be less than the saturation limit of different types of skin.

The monolithic design, for example, typically has only three layers: the adhesive layer, a polymer matrix containing the compound, and a water-proof backing. This design brings a saturating amount of drug to the skin. Thereby, delivery is controlled by the skin. As the drug amount decreases in the patch to below the saturating level, the delivery rate falls.

The active agents of the present invention can be useful, for instance, in the treatment, prevention, suppression of pain, anxiety, depression, and PTSD and may be used in combination with other compounds or with other drugs that are useful in the treatment, prevention, suppression of pain, anxiety, depression, or PTSD. Such other drugs may be administered, by a route and in an amount commonly used therefore, contemporaneously or sequentially with a compound of the invention. When the active agent is used contemporaneously with one or more other drugs, a pharmaceutical composition in unit dosage form containing such other drugs and the compound is preferred. When used in combination with one or more other active ingredients, the compound of the present invention and the other active ingredients may be used in lower doses than when each is used singly. Accordingly, the pharmaceutical compositions of the present invention include those that contain one or more other active ingredients, in addition to the compounds disclosed above.

The pharmaceutically or physiologically acceptable salts include, but are not limited to, a metal salts such as sodium salt, potassium salt, lithium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like.

The pharmaceutically active agents (e.g., FAAH inhibitors, MGL inhibitors, COX-2 inhibitors, cannabinoid receptor agonists, opioids, NSAIDs, anandamide transport inhibitors, and PPARα agonists) to be used according to the invention may be administered by a variety of routes. These routes include, but are not limited to, the oral route, the intravenous route, and the dermal routes of administration. They may be administered locally (e.g., near the site of the pain or the primary lesion or dysfunction) or systemically. When one or more active agents are to be administered, they may be administered concurrently or at different times. They may be administered on the same or different schedules (e.g., according to the biological half-times in the body or their individual duration of action). They may be administered together via one pharmaceutical composition or via separate pharmaceutical compositions.

In some other aspects, the invention provides methods of 1) treating pain or stress-induced analgesia in a mammalian subject in need thereof; 2) methods for treating a stress-induced disorder or condition in a mammalian subject in need thereof; 3) methods for enhancing or potentiating stress-induced analgesia in a mammalian subject in need thereof, and 4) methods of producing analgesia in a patient in need thereof, wherein the patient is tolerant to morphine which use compounds according to the invention:

Accordingly, the invention provides a method of treating pain or stress-induced analgesia in a mammalian subject in need thereof comprising administering to the subject at least one compound selected from the group consisting of: 2-arachidonylglycerol hydrolysis inhibitors, analgesics, opioids, NSAIDs, FAAH inhibitors, PPARα agonists, anandamide transport inhibitors, CB1 receptor agonists, anxiolytics, and monoacylglycerol lipase (MGL) inhibitors, including, particularly the compounds for use according to the invention as disclosed herein. In yet other embodiments, the disorder is post-traumatic stress disorder, an anxiety disorder, or depression. In some preferred embodiments, the subject is human. In other preferred embodiments, a 2-AG modulator, an MGL inhibitor, or a selective MGL inhibitor is administered.

The invention provides a method for treating a stress-induced disorder or condition in a mammalian subject in need thereof, comprising administering to the subject a therapeutically effective amount of at least one compound selected from the group consisting of: 2-arachidonylglycerol hydrolysis inhibitors, analgesics, opioids, NSAIDs, FAAH inhibitors, PPARα agonists, anandamide transport inhibitors, CB1 receptor agonists, anxiolytics, antidepressants, 2-AG hydrolysis inhibitors and monoacylglycerol lipase (MGL) inhibitors including, particularly the compounds for use according to the invention as disclosed herein. In yet other embodiments, the disorder is post-traumatic stress disorder, an anxiety disorder, or depression. In some preferred embodiments, the subject is human. In other preferred embodiments, a 2-AG modulator, an MGL inhibitor, or a selective MGL inhibitor is administered.

The invention provides a method for enhancing or potentiating stress-induced analgesia in a mammalian subject in need thereof, comprising administering at least one compound that stimulates central nervous system cannabinoid receptors. In some embodiments, the compound is selected from the group consisting of: 2-arachidonylglycerol hydrolysis inhibitors, FAAH inhibitors, anandamide transport inhibitors, CB1 receptor agonists, and monoacylglycerol lipase (MGL) inhibitor, including, particularly the compounds for use according to the invention as disclosed herein. In some preferred embodiments, the subject is human. In additional preferred embodiments, a 2-AG modulator, an MGL inhibitor, or a selective MGL inhibitor is administered.

In one other aspect, the invention provides a method of producing analgesia in a patient in need thereof, wherein the patient is tolerant to morphine, comprising stimulation of central nervous system cannabinoid receptors. In some further embodiments, the stimulation of central nervous system cannabinoid receptors is the result of administration of at least one compound selected from the group consisting of: 2-arachidonylglycerol hydrolysis inhibitors, FAAH inhibitors, anandamide transport inhibitors, CB1 receptor agonists, and monoacylglycerol lipase (MGL) inhibitors, including, particularly the compounds for use according to the invention as disclosed herein. In preferred embodiments, the subject is human. In additional preferred embodiments, a 2-AG modulator, an MGL inhibitor, or a selective MGL inhibitor is administered.

In an additional aspect the invention provides a method of producing analgesia in a patient in need thereof, wherein the patient is tolerant to morphine, comprising administration of at least one compound selected from the group consisting of: 2-arachidonylglycerol hydrolysis inhibitors, FAAH inhibitors, anandamide transport inhibitors, CB1 receptor agonists, and monoacylglycerol lipase (MGL) inhibitors including, particularly, the compounds for use according to the invention as disclosed herein. In some preferred embodiments, the subject is human. In additional preferred embodiments, a 2-AG modulator, an MGL inhibitor, or a selective MGL inhibitor is administered A 2-AG modulator is a compound which increases or selectively increases the activity of 2-AG at the CB1 receptor in vivo or in vitro.

The following examples are provided to illustrate, and not to limit, the invention.

### EXAMPLES

### Example 1

### Role of 2-AG in Stress-Induced Analgesia and Modulation by MGL and FAAH Inhibitors

Acute stress suppresses pain by activating brain pathways that engage both opioid and non-opioid mechanisms. Injection of CB1 cannabinoid receptor antagonists into the periaqueductal gray matter (PAG) of the midbrain is shown here to prevent non-opioid stress-induced analgesia. In this region, stress is shown to elicit the rapid formation of two endocannabinoids, 2-arachidonoylglycerol (2-AG) and anandamide. In addition, newly developed inhibitors of the 2-AG-deactivating enzyme monoacylglycerol lipase (MGL) is shown to selectively increase 2-AG levels and, when injected into the PAG, magnify stress-induced analgesia in a CB1-dependent manner. Fatty-acid amide hydrolase inhibitors, which selectively elevate anandamide levels, are shown to exert similar effects. The results indicate that release of both 2-AG and anandamide in the PAG mediates opioid-independent stress-induced analgesia, and identify MGL as a novel drug target.

To study opioid-independent stress analgesia brief, continuous electric foot shock were delivered to rats and their post-stress sensitivity to pain using the tail-flick test quantified. As demonstrated in previous studies (Lewis, J. W. et al., Science 208:623-625 (1980); Lewis, J. W. et al., Science 217:557-559 (1982); Grau, J. W. et al., Science 213:1409-1411 (1981) Terman, G. W. et al., Brain Res. 368:101-106 (1986)) this stimulation protocol caused a profound antinociceptive response that was insensitive to intraperitoneal (i.p.) injection of a maximally effective dose of the opiate antagonist naltrexone (14 mg-kg⁻¹, i.p.) (FIG. 1a). The response was almost abolished, however, by systemic administration of the CB₁-selective antagonist/inverse agonist rimonabant (SR141617A, 5 mg-kg⁻¹, i.p.) (FIG. 1a) or its analogue AM251 (5 mg-kg⁻¹, i.p.) (Supplementary FIG. 1), but not by the CB₂ antagonist SR144528 (5 mg-kg⁻¹, i.p.) (FIG. 1a). The effects of the CB₁ antagonists cannot be attributed to changes in basal nociceptive threshold because, in the absence of the stressor, the drugs failed to alter tail-flick latencies (FIG. 1b and Supplementary FIG. 1).

If CB₁ receptor activation is required for the expression of non-opioid stress analgesia, as these findings indicate, then this response should be reduced in animals rendered tolerant to the antinociceptive effects of cannabinoid agonists. Consistent with this prediction, rats treated chronically with the cannabinoid agonist WIN55, 212-2 (10 mg-kg⁻¹ i.p., once daily for 14 days) displayed, along with the expected blunting of acute CB₁-dependent antinociception (FIG. 1c inset), a marked reduction in the levels of stress-induced analgesia assessed 24 h after the last agonist injection (FIG. 1c). The possibility that this decrement might be due to downstream regulatory changes in opioid tone is unlikely, based on two complementary observations. First, rats tolerant to WIN55, 212-2 showed no deficit in their antinociceptive response to morphine (2.5 mg-kg⁻¹ s.c.), which was in fact slightly greater than that observed in vehicle-treated controls (mean tail-flick latency after morphine: control rats, 5.33±0.21 s; cannabinoid tolerant rats, 6.09±1.93 s; F_{1,11}=6.90 P<0.03; n=6-7). Second, in accord with previous results (Terman, G. W. et al., Brain Res. 368:101-106 (1986)), rats tolerant to morphine (10 mg-kg⁻¹ s.c. once daily for 7 days)(FIG. 1d, inset) displayed a normal degree of non-opioid stress analgesia (FIG. 1d).

The midbrain PAG serves key functions in both the descending control of pain (Millan, M. J. Prog. Neurobiol. 66:355-474 (2002)) and the antinociceptive actions of cannabinoid agonists (Martin, W. J. et al., Life Sci. 56:2103-2109 (1995)). Therefore, the effects of blockade of CB₁ receptors in this structure on stress analgesia were examined. Rimonabant (2 nmol) significantly reduced stress-induced analgesia when microinjected into the dorsolateral subdivision of the PAG (FIG. 1e,g), which has been specifically linked to non-opioid stimulation-produced analgesia (Cannon, J. T. et al., Brain Res. 243:315-321 (1982); Walker, J. M. et al., Proc. Natl. Acad. Sci. U.S.A. 96:12198-12203 (1999)), but was inactive after injection into the ventrolateral PAG (FIG. 1f,h). The drug was also ineffective when injected into the lateral ventricle, indicating that its actions were not due to diffusion to distal sites (Supplementary FIG. 2). These results are consistent with the presence of CB₁ binding sites and CB₁-immunoreactive fibers throughout the dorsal midbrain (Herkenham, M. et al. J. Neurosci. 11:563-583 (1991); Tsou, K. et al., Neuroscience 83:393-411 (1998)) and suggest that endocannabinoid release and/or intrinsic CB₁ receptor activity in the PAG contribute in important ways to stress analgesia.

At least two lipid molecules, anandamide (Devane, W. A. et al. Science 258:1946-1949 (1992)) and 2-arachidonoylglycerol (2-AG) (Mechoulam, R. et al. Biochem. Pharmacol. 50:83-90 (1995)), meet the defining criteria of an endocannabinoid. Both are produced by neurons in an activity-dependent manner, engage CB₁ receptors with high affinity, and are rapidly eliminated through regulated transport and intracellular hydrolysis (Piomelli, D. Nat. Rev. Neurosci. 4:873-884 (2003)). In neurons, the hydrolysis of anandamide and 2-AG is catalyzed by two distinct serine hydrolases: fatty-acid amide hydrolase (FAAH) cleaves anandamide along with other lipid amides (Cravatt, B. F. et al. Nature 384:83-87 (1996)), while monoacylglycerol lipase (MGL) hydrolyzes 2-AG and other monoacylglycerols (Dinh, T. P. et al. Proc. Natl. Acad. Sci. USA 99:10819-10824 (2002)). To determine whether endocannabinoid release participates in stress analgesia, anandamide and 2-AG levels in dorsal midbrain fragments, which included the entire PAG, were measured in rats sacrificed before or various times after foot shock. Liquid chromatography/mass spectrometry (LC/MS) analyses revealed that anandamide and 2-AG are present in the tissue fragments, as determined by the occurrence of diagnostic ions of appropriate mass-to-charge ratio (m/z) and retention time (FIG. 2a,b). Importantly, midbrain 2-AG levels markedly increased within 2 min of shock delivery, returning to baseline values approximately 15 min later (FIG. 2c). This response preceded a sustained increase in anandamide levels, which peaked 7-15 min after the shock (FIG. 2c). No such changes were observed in the occipital cortex (FIG. 2d), a brain region that contains CB₁ receptors (Herkenham, M. et al. J. Neurosci. 11:563-583 (1991)) but is not part of the stress analgesia circuit.

The rapid post-stress accumulation of 2-AG in the midbrain supports the possibility that endocannabinoid release, rather than intrinsic CB₁ receptor activity, is responsible for stress analgesia. A corollary of this hypothesis is that selective inhibitors of the 2-AG-hydrolyzing enzyme MGL should heighten the intrinsic actions of 2-AG and, by doing so, should enhance its analgesic effects.

A collection of substituted carbamate derivatives in which selective FAAH inhibition had been achieved by mimicking the flexible fatty-acid chain of anandamide with an isosteric, but more rigid biphenyl group (FIG. 3 a) (Kathuria, S. et al., Nature Med. 1:76-81 (2003); Mor, M. et al. J. Med. Chem. 47:4998-5008 (2004)) were examined for MGL inhibitory activity. This screening revealed that, while O-biphenyl carbamates (FIG. 3a, 1, URB597; 2, URB524) inhibit the activity of FAAH, but not MGL, N-biphenyl carbamates (FIG. 3a, 3, URB602) display an opposite selectivity (FIG. 3b,c). Thus, inversion of the biphenyl and alkyl substituents across the carbamate group removed FAAH-inhibitory activity and enabled MGL inhibition to occur.

In broken cell preparations, URB602 inhibited native rat brain MGL with a half-maximal concentration (IC₅₀) of 28±4 μM (FIG. 3b). Kinetic analyses indicated that the inhibition occurred through a non-competitive mechanism: without URB602, the apparent Michaelis constant (K_{M}) of MGL for 2-AG was 24.0±1.7 μM and the maximum velocity (Vₘₐₓ) was 1814±51 nmol-min-mg⁻¹ protein; with URB602, the apparent K_{M} was 20.0±0.4 μM and the Vₘₐₓ was 541±20 nmol-min-mg⁻¹ protein (n=4). In contrast to its effects on MGL, URB602 did not affect the activities of several lipid-metabolizing enzymes, including diacylglycerol lipase (DGL) (which catalyzes 2-AG formation) (Stella, N. et al., Nature 388:773-778 (1997)), cyclooxygenase (Cox)-2 (which has been implicated in 2-AG metabolism) (Kozak, K. R. et al., Curr. Pharm. Des. 10:659-667 (2004)), phospholipase C (PLC), and PLD (Supplementary Table 1). Furthermore, the compound did not significantly influence the binding of [³H]-WIN55212-2 to CB₁ or CB₂ receptors (IC₅₀≧10 μl) or [³⁵S]-GTP-γ-S to rat cerebellar membranes [half-maximal effective concentration (EC₅₀)>100 μM] (data not shown). To examine whether URB602 protects 2-AG from degradation in intact brain cells, we exposed organotypic slice cultures of rat forebrain to the inhibitor and measured endocannabinoid content by LC/MS. Incubation with URB602 (100 μM) significantly increased basal 2-AG levels and enhanced 2-AG accumulation elicited by the Ca²⁺-ionophore ionomycin (2 μM) (FIG. 3d). Underscoring the specificity of this effect, URB602 did not modify anandamide levels (FIG. 3e), which were markedly elevated by the FAAH inhibitor URB597 (1 μM) (FIG. 3e).

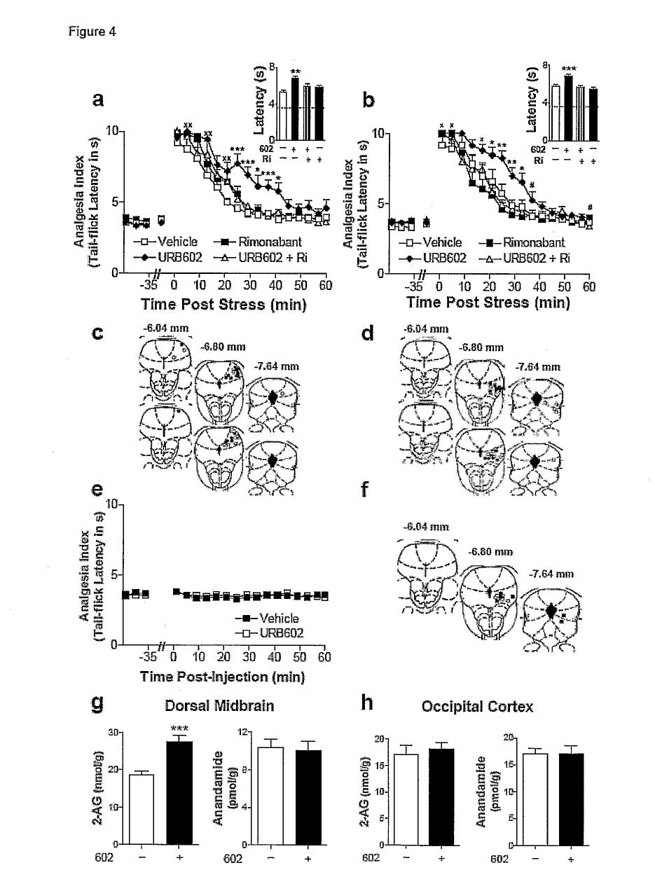

Because of its relatively low potency, URB602 may not be as suitable for systemic administration or much higher dosages may be required. However, the selectivity of this compound for MGL enabled us to test its effects after site-specific injection into the brain. Microinjections of URB602 (0.1 nmol) into the dorsolateral or ventrolateral PAG enhanced stress-induced analgesia (FIG. 4a-d) without altering basal nociceptive thresholds in non-shocked rats (FIG. 4e,f). This effect was likely due to the accumulation of non-hydrolysed 2-AG in the PAG, for three reasons. First, it was prevented by local co-administration of the CB₁ antagonist rimonabant, at a dose (0.2 nmol) that was insufficient to reverse stress analgesia (FIG. 4a,b). Second, it was mimicked by the non-selective MGL inhibitor methylarachidonylfluorophosphonate (2.6 nmol) (Dinh, T. P. et al., Mol. Pharmacol. 66:1260-1264 (2004)), whose effects also were blocked by rimonabant (Supplementary FIG. 3). Third, it was accompanied by an elevation in midbrain 2-AG levels: 25 min after the shock, when the antinociceptive effect of URB602 was at its peak (FIG. 4 a,b), 2-AG content was significantly higher in midbrain fragments of URB602-treated rats than in those of untreated controls (FIG. 4g). Importantly, anandamide levels were identical in the two groups (FIG. 4g), further highlighting the selectivity of URB602 for MGL. As expected, 2-AG and anandamide concentrations in the occipital cortex were not affected by URB602 injection into the PAG (FIG. 4h). Collectively, these results indicate that URB602 is a selective inhibitor of MGL, which enhances both 2-AG accumulation and stress analgesia.

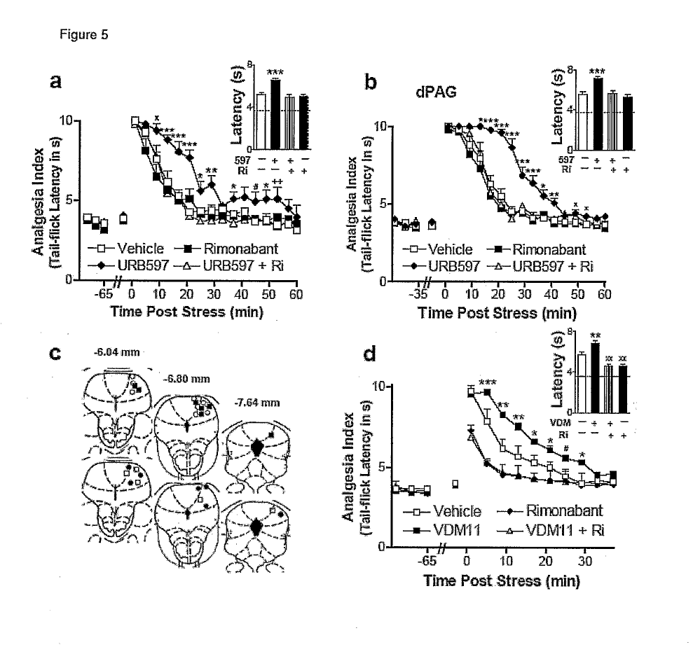

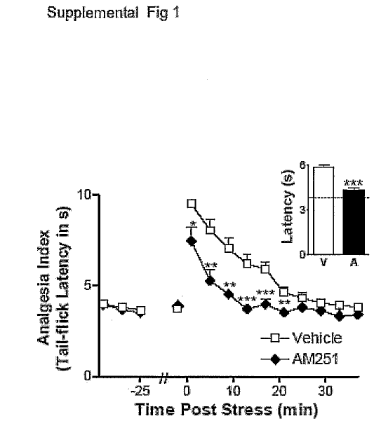

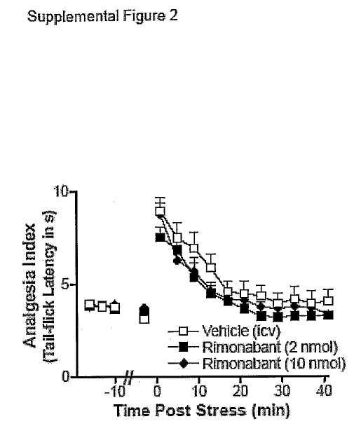

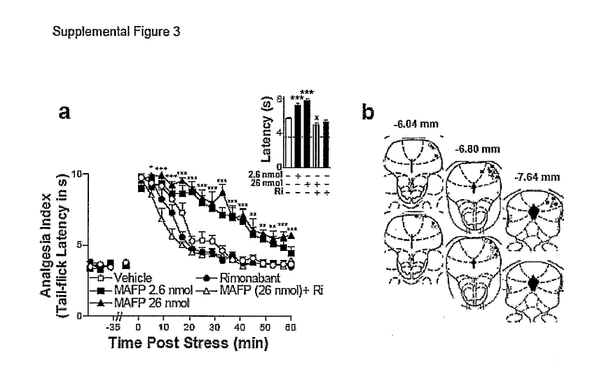

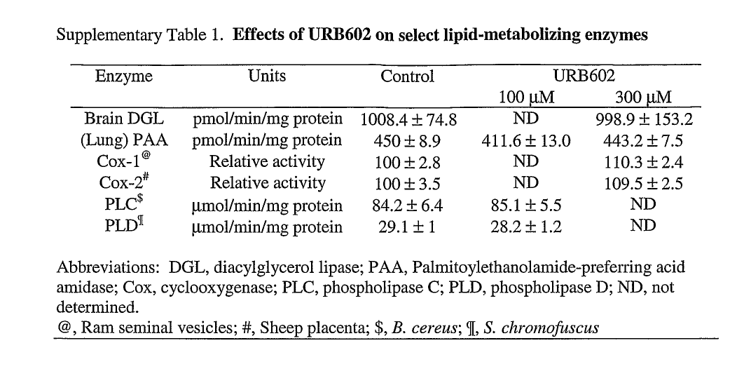

Midbrain anandamide levels increase after stress subsequent to earlier increases in 2-AG accumulation (FIG. 2c). The role of this response was examined in stress analgesia by administering the FAAH inhibitor URB597 either by systemic injection—at a dose that was previously shown to selectively block anandamide degradation in the brain (Kathuria, S. et al., Nature Med. 1:76-81 (2003)) (0.3 mg-kg⁻¹, i.p) (FIG. 5a)—or by microinjection into the dorsolateral PAG (0.1 nmol) (FIG. 5b,c). Irrespective of the route of administration, URB597 caused a significant potentiation of stress-induced analgesia, which was prevented by treatment with rimonabant (1 mg-kg⁻¹ i.p.; 0.2 nmol in the PAG) (FIG. 5a,b). In contrast, URB597 did not modify basal nociceptive thresholds (FIG. 5a,b). Intraperitoneal administration of the anandamide transport inhibitor VDM11 (10 mg-kg⁻¹ i.p.) (De Petrocellis, L. et al., FEBS Lett. 483:52-56 (2000)) exerted a similar effect, which also was blocked by rimonabant (2 mg-kg⁻¹ i.p.)(FIG. 5d).

These finding indicate that 1) stress activates an endocannabinoid analgesic mechanism that suppresses pain, independently of endogenous opioids, through a cannabinoid CB1 mechanism, 2) 2-arachidonoylglycerol (2-AG) and anandamide are the endogenous mediators of stress analgesia, 3) endogenous 2-AG, an endocannabinoid, serves naturally to suppress sensitivity to pain, 4) selective inhibition of fatty acid amide hydrolase or the anandamide membrane transporter enhances stress analgesia, and 5) selective inhibition of monoacylgylcerol lipase, the enzyme responsible for hydrolysis of 2-AG, enhances stress analgesia and accumulation of endogenous 2-AG. This invention also provides a selective inhibitor of monoacylglycerol lipase, URB602 and teaches that inhibition of 2-AG hydrolysis enhances both stress analgesia and brain levels of 2-AG without altering levels of anandamide.

These results indicate that the concerted release of two endocannabinoid lipids, 2-AG and anandamide, mediates non-opioid stress analgesia. Endocannabinoids generated by stress may interact with presynaptic CB₁ receptors in the PAG (Herkenham, M. et al. J. Neurosci. 11:563-583 (1991); Tsou, K. et al., Neuroscience 83:393-411 (1998))—as previously shown for synthetic CB₁ agonists (Vaughan, C. W. et al., Mol. Pharmacol. 57:288-295 (2000))—to regulate local glutamatergic and GABAergic transmission, ultimately disinhibiting descending pain control pathways. Three points are noteworthy. First, the fact that opioid antagonists and opiate tolerance do not affect endocannabinoid-dependent stress analgesia implies that this process does not depend on opioid activity. The reverse may not be true, however, as mutant mice lacking CB₁ receptors display reduced opioid-mediated responses to stress (Valverde, O. et al., Eur. J. Neurosci. 12:533-539 (2000)). Second, the residual antinociception observed in the presence of maximally effective doses of CB₁ antagonists leaves open the possibility that additional mediators of stress analgesia remain to be discovered. Third, even though stress triggers the formation of both 2-AG and anandamide in the midbrain, the two endocannabinoids are released with strikingly dissimilar time-courses. This finding underscores the existence of a functional differentiation between these signalling molecules (Piomelli, D. Nat. Rev. Neurosci. 4:873-884 (2003)) pointing to the possibility that they may act in a coordinated manner to modulate temporally and/or spatially distinct processes in the PAG and other brain regions. The ability of both MGL and FAAH inhibitors to magnify endocannabinoid stress analgesia supports this idea and highlights the significance of these enzymes as targets for analgesic and anti-stress drugs.

### Methods

Chemicals. URB597, URB524 and [²H₄]-Anandamide were synthesized as described^{24,25,32,33} and URB602 (biphenyl-3-yl carbamic acid cyclohexyl ester) by reacting diimidazole-1-ylmethanone with biphenyl-3-yl amine in CH₃CN in the presence of DMAP and, subsequently, with cyclohexanol. Other chemicals were from the National Institute on Drug Abuse (rimonabant, SR144528), Sigma-Aldrich (WIN55, 212-2, morphine sulfate), (2 Tocris (VDM11, AM251), and Cayman ([H₈]-2-AG, URB597). Drugs were dissolved in a vehicle of emulphor:ethanol:saline (1:1:8 by volume) or dimethylsulfoxide (DMSO). Injection volumes were 1 ml-kg⁻¹ for systemic administration, 10 μl for intracerebroventricular administration, and 1 μl for PAG microinjection. Rimonabant, SR144528, naltrexone and AM251 were administered by intraperitoneal (i.p.) injection 25 min before the tests or by microinjection 10 min before the tests. URB602, URB597, VDM11 and MAFP were administered by i.p. (65 min before tests) or intracranial (35 min before tests) injection in the presence or absence of rimonabant.

Animals. Adult male Sprague-Dawley rats were used for in vivo experiments and Wistar rats for enzymes assays and tissue cultures. All procedures were approved by the institutional animal care and use committee and followed guidelines of the International Association for the Study of Pain.

Brain slice cultures. Brain slices were cultured from Wistar rats. Pups were sacrificed on post-natal day 5 by decapitation following cryo-anaesthesia. Brains were removed and cut (0.4 mm-thick coronal slices) using a vibratome in a bath of ice-cold high-glucose Dulbecco's Modified Eagle's Medium (Gibco). Hemispheres were placed on Millicell culture inserts (Millipore) in 6-well plates with serum-based culture medium (1.5 ml) composed of basal Eagle medium with Earle's salts (100 ml), Earle's balanced salt solution (50 ml), heat-inactivated horse serum (50 ml), L-glutamine (0.2 mM, 1 ml) and 50% glucose (2 ml) (Gibco). Slices were maintained at 37° C. with 5% CO₂ for 7 days before use.

Lipid extractions and LC/MS analyses. Ex vivo experiments. Rats were habituated to the guillotine for at least 7 days prior to the experiment and sacrificed them either before or various times (2, 7, 15 and 25 min) after a 3-min foot shock (n=10 per group). The brains were rapidly removed, dissected and stored frozen (−80° C.) until lipid extraction. In vitro experiments. The medium of slice cultures was removed and replaced it with DMEM (1 ml) containing URB602 (100 μM), URB597 (1 μM) or vehicle (0.1% DMSO) and incubated the slices at room temperature for 10 min. In some experiments, slices were incubated with ionomycin (2 μM) in DMEM for additional 15 min. Reactions were stopped and washed with ice-cold 50% methanol (1 ml). Slices were collected in the same medium (0.2 ml) and homogenized. Brain tissue (≈50 mg) and slice homogenates were suspended in methanol (2 ml) containing [²H]-containing internal standards (25 pmol). Lipids were extracted in methanol-chloroform-H₂O (1:2:0.25). The organic phase was recovered, evaporated to dryness, reconstituted in chloroform/methanol (1:3, 80 μL) and subjected to LC/MS analysis as described (Giuffrida, A. et al., Anal. Biochem. 280:87-93 (2000)).

Enzyme assays. Cell fractions were prepared from Wistar rat brain homogenates, and assayed cytosol MGL activity and membrane FAAH activity using 2-monooleoylglycerol[glycerol-1,2,3-³H] (ARC, St. Louis, Mo., 20 Ci/mmol), and anandamide[ethanolamine-³H] (ARC, St. Louis, Mo.), 60 Ci/mmol) respectively, as substrates (Dinh, T. P. et al. Proc. Natl. Acad. Sci. USA 99:10819-10824 (2002); Kathuria, S. et al., Nature Med. 1:76-81 (2003)).

Surgery. Stainless-steel guide cannulae were implanted in the left lateral ventricle or PAG (dorsolateral or ventrolateral), under pentobarbital/ketamine anaesthesia 3-7 days prior to testing. Cannulae placements were verified in Nissl-stained sections or by post mortem injection of Fast-green dye. Analyses were restricted to animals exhibiting dye spread throughout the ventricular system.

Tolerance Induction. Sprague-Dawley rats received daily i.p. injections of vehicle or WIN55212-2 for 2 weeks (10 mg-kg⁻¹ once daily). Morphine antinociception (2.5 mg-kg⁻¹ s.c. on day 15) was assessed in separate groups treated chronically with WIN55212-2 or vehicle. Separate groups received subcutaneous (s.c.) injections of vehicle or morphine (10 mg-kg⁻¹ once daily for 7 days). Post-injection tail-flick latencies were measured on days 2, 7 and 14 (chronic WIN55212-2 study) or days 1 and 7 (chronic morphine study) to confirm that the injection paradigm induced tolerance to the antinociceptive effects of each agonist prior to administration of the stressor. 24 h after the last injection, rats were subjected to foot shock, and stress analgesia was quantified. Ceiling tail-flick latencies were 15 s.

Analgesia tests. Foot shock (0.9 mA, AC current, 3 min) was administered to Sprague-Dawley rats using a Lafayette grid-shock apparatus. Withdrawal latencies in the radiant heat tail-flick test (Martin, W. J. et al., J. Neurosci. 16:6601-6611 (1996); Walker, J. M. et al., Proc. Natl. Acad. Sci. U.S.A. 96:12198-12203 (1999)) were measured at 2-min intervals before (baseline) and after foot shock, and calculated for each subject in 2-trial blocks. Removal of the tail from the heat source automatically terminated application of thermal stimulation. Tail-flick latencies were monitored over 4 min immediately prior to exposure to the stressor to evaluate changes in nociceptive thresholds induced by pharmacological manipulations. Ceiling tail-flick latencies were 10 s except where noted. Tail-flick latencies, measured at baseline or prior to administration of the stressor, did not differ between groups in any study.

Data analyses. Results were analyzed using ANOVA, repeated measures ANOVA and Fisher's PLSD post hoc tests. P<0.05 was considered significant.

Supplemental Methods. CB₁ and CB₂ binding assays were conducted in rat cerebellar membranes and CB₂-overexpressing CHO cells (Receptor Biology-Perkin Elmer, Wellesley, Mass.), respectively, using [³H]WIN-55212-2 (NEN-Dupont, Boston, Mass., 40-60 Ci/mmol) as a ligand. We measured phospholipase C and phospholipase D activities at 37° C. for 15 min in 35 mM Tris-maleate buffer (0.5 ml, pH 7.3) containing calcium chloride (5 mM), fatty acid-free BSA (2 mg-ml-1, Sigma), phospholipase C (B. cereus, 1 U; Sigma) or phospholipase D (S. chromofuscus, 10 U, Sigma). Phosphatidylcholine[³H]methylcholine (8 mM, ARC, 60 ci/mmol, 20,000 dpm) was used as a substrate. Reactions were terminated by adding chloroform:methanol (1:1, 1 ml). Radioactivity was determined by liquid scintillation counting. DGL activity was measured at 37° C. for 30 min in 0.5 ml Tris buffer (50 mM, pH 7.5), rat brain protein (800 g, supernatant, 100 mg protein) and [³H]dioleoylglycerol (50 μM, 75,000 dpm; ARC, St. Louis, Mo.). After stopping the reactions with chloroform/methanol (1:1, 1 ml), we collected 0.5 ml of organic layer and added 5 μg of diolein, 5 μg monoleoylglycerol and 12.5 μg oleic acid and dried under a stream of nitrogen. Thin-layer chromatography analyses were carried out on silica gel G plates, eluted with a solvent system consisting of chloroform/methanol/ammonium hydroxide (85:15:0.1). Lipids were visualized by iodine staining, and the bands scraped. Radioactivity was determined by liquid scintillation counting. We performed cyclooxygenase (Cox) assays with a commercial kit using purified enzymes (Cox-1 from ram seminal vesicles, Cox-2 human recombinant) (Cayman Chemicals, Ann Arbor, Mich.).

Each publication, patent application, patent, and other reference cited herein is incorporated by reference in its entirety to the extent that it is not inconsistent with the present disclosure. In particular, all publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for identifying a compound for treating pain or modulating stress analgesia, comprising the step of determining whether the compound inhibits the catalytic activity of a monoacylglycerol lipase (MGL), wherein the compound is identified for use in treating pain or modulating stress analgesia when the compound is determined to inhibit the catalytic activity of MGL.

2. The method of claim 1, wherein the MGL is mammalian.

3. The method of claim 1, wherein the contacting is in vitro.

4. The method of claim 1, wherein the MGL is brain tissue MGL.

5. The method of claim 1, wherein the method measures the hydrolysis of 2-arachidonylglycerol by MGL.

6. A method for treating pain or modulating stress-induced analgesia in a mammalian subject in need thereof, comprising administering to the subject a therapeutically effective amount of a compound which was identified for use in treating pain or modulating stress-induced analgesia by the method of claim 1.

7. The method of claim 6, wherein the pain is neuropathic pain.

8. The method of claim 6, wherein the pain is inflammatory pain.

9. The method of claim 6, wherein the pain is a chronic pain.

10. The method of claim 6, wherein a second analgesic or anti-nociceptive agent is administered to the subject.

11. The method of claim 6, wherein the subject is human.

12. The method of claim 6, wherein stress-induced analgesia is modulated in the subject.

13. The method of claim 6, wherein the compound is of Formula I: 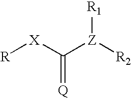
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 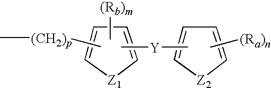
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached.

14. A pharmaceutical composition comprising a therapeutically effective amount of a selective MGL inhibitor for the treatment of pain in a mammalian subject.

15. The composition of claim 14, further comprising at least one additional agent selected from the group consisting of analgesics, NSAIDs, opioids, FAAH inhibitors, PPARα agonists, anandamide transport inhibitors, and CB1 receptor agonists.

16. The composition of claim 14, wherein the subject is human.

17. The composition of claim 14, wherein the compound is of Formula I: 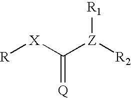
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 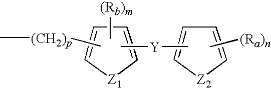
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached, and
wherein the compound inhibits MGL with an IC₅₀ of less than 1 micromolar.

18. The pharmaceutical composition of claim 14, wherein the compound is 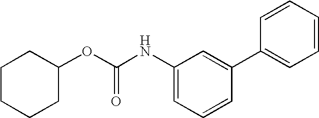
or a pharmaceutically acceptable salt thereof.

19. The pharmaceutical composition of claim 18, further comprising at least one additional agent selected from the group consisting of analgesics, opioids, NSAIDs, FAAH inhibitors, PPARα agonists, anandamide transport inhibitors, and CB1 receptor agonists.

20. A method of treating pain in a mammalian subject in need thereof, comprising administering to the subject a selective MGL inhibitor.

21. The method of claim 20, wherein the subject is tolerant to opioid anti-nociception.

22. The method of claim 20, wherein a second pharmaceutical agent selected from the group consisting of analgesics, opioids, NSAIDs, FAAH inhibitors, PPARα agonists, anandamide transport inhibitors, CB1 receptor agonists, anxiolytics, antidepressants is administered.

23. The method of claim 20, wherein the compound is of Formula I: 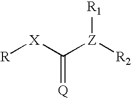
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 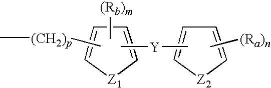
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached, and
wherein the compound inhibits MGL with an IC₅₀ of less than 1 micromolar.

24. The method of claim 20, wherein the compound is 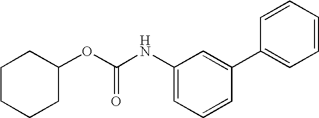
or a pharmaceutically acceptable salt thereof.

25. The method of claim 20, wherein the pain is neuropathic pain.

26. The method of claim 20, wherein the pain is inflammatory pain.

27. The method of claim 20, wherein the pain is a chronic pain.

28. A method for enhancing or potentiating stress-induced analgesia in a mammalian subject in need thereof, comprising administering at least one compound that increases stimulation of central nervous system cannabinoid receptors.

29. The method of claim 28, wherein the compound is an inhibitor of monoacyl glycerol lipase.

30. The method of claim 28, wherein the compound is a CB1 receptor agonist.

31. The method of claim 28, wherein the compound is an inhibitor of the hydrolysis or transport of 2-arachidonoylglycerol.

32. The method of claim 28, wherein the compound is of Formula 1: 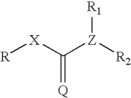
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 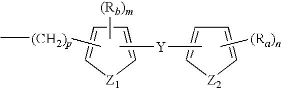
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached.

33. The method of claim 32, wherein the compound is 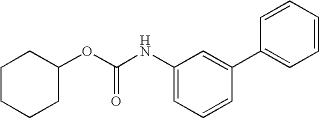
or a pharmaceutically acceptable salt thereof.

34. A method of inhibiting monacylglycerol lipase by contacting the monacylglycerol lipase with a compound of Formula I: 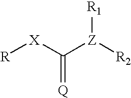
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 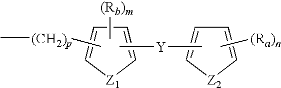
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached.

35. The method of claim 34, wherein the compound is 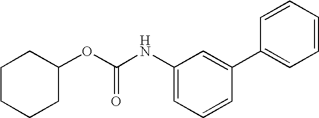
or a pharmaceutically acceptable salt thereof.

36. The method of claim 34, wherein the compound has an IC₅₀ for inhibiting MGL of less than 1 micromolar.

37. The method of claim 34, wherein the compound is a selective MGL inhibitor.

38. A method of claim 34, wherein the contacting is in vitro.

39. The method of claim 34, wherein the contacting is in vivo.

40. A method for identifying a compound for treating a stress-induced disorder, comprising the step of determining whether the compound is an inhibitor of mammalian brain monoacylglycerol lipase (MGL), wherein the compound is identified as being useful in treating a stress-induced disorder when it is determined to be an inhibitor of the MGL.

41. The method of claim 40, wherein the MGL is human MGL.

42. A method for treating a stress-induced disorder or condition in a mammalian subject in need thereof, comprising administering to the subject a therapeutically effective amount of a compound which was identified by the method of claim 40.

43. The method of claim 42, wherein the disorder is Post Traumatic Stress Disorder (PTSD).

44. The method of claim 42, wherein the disorder is an anxiety disorder.

45. The method of claim 42, wherein the disorder is depression.

46. The method of claim 42, wherein the subject is human.

47. The method of claim 42, further comprising administering to the subject at least one additional agent selected from the group consisting of anti-depressants and anxiolytic agents.

48. A method of treating a stress-induced disorder or condition in a mammalian subject in need thereof, comprising administering a monoacylglycerol inhibitor to the subject.

49. The method of claim 48, wherein the disorder is Post Traumatic Stress Disorder (PTSD).

50. The method of claim 48, wherein the disorder is an anxiety disorder.

51. The method of claim 48, wherein the disorder is depression.

52. The method of claim 48, wherein the subject is human.

53. The method of claim 48, further comprising administering to the subject at least one additional agent selected from the group consisting of anti-depressants and anxiolytic agents.

54. The method of claim 48, wherein the compound is of Formula I: 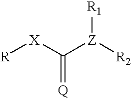
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 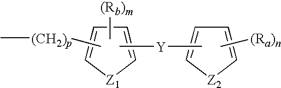
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached, and
wherein the compound inhibits MGL with an IC₅₀ of less than 1 micromolar.

55. The method of claim 48, wherein the compound is 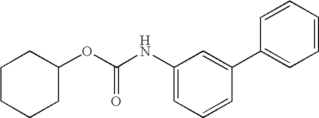
or a pharmaceutically acceptable salt thereof.

56. A pharmaceutical composition comprising a selective MGL inhibitor in a therapeutically effective amount for the treatment of a stress-induced disorder or condition in a mammalian subject.

57. The composition of claim 56, wherein the subject is human.

58. The composition of claim 56, wherein the compound is of Formula I: 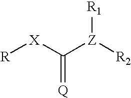
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 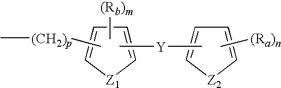
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached, and
wherein the compound inhibits MGL with an IC₅₀ of less than 1 micromolar.

59. The pharmaceutical composition of claim 56, wherein the compound is 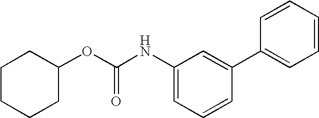
or a pharmaceutically acceptable salt thereof.

60. The pharmaceutical composition of claim 56, further comprising at least one additional agent selected from the group consisting of analgesics, opioids, NSAIDs, FAAH inhibitors, PPARα agonists, anandamide transport inhibitors, and CB1 receptor agonists.

61. The use of a compound in the manufacture of a medicament for treating pain or a stress-induced disorder, wherein the compound is of Formula I: 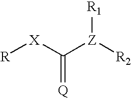
in which X is CH₂, NH, O, or S; Q is O or S; Z is O or N, with the proviso that when Z is O, one of R₁ and R₂ is absent; and R is a moiety selected from the group consisting of substituted or unsubstituted alkyl, substituted or unsubstituted heteroalkyl, substituted or unsubstituted cycloheteroalkyl; substituted or unsubstituted aryl; substituted or unsubstituted biphenyl, substituted or unsubstituted naphthyl, and substituted or unsubstituted phenyl; substituted or unsubstituted terphenyl; substituted or unsubstituted cycloalkyl, substituted or unsubstituted heteroaryl, and 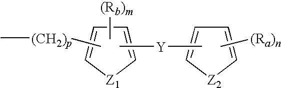
wherein p is a number from 0 to 3; m is a number from 0 to 4, and n is a number from 0 to 5, Z₁ and Z₂ are same or different and are independently a divalent radical selected from the group consisting of —O—, —S—, —N(R₅)—, —C(R₆)═C(R₇)—, and —N═C(R₆)— wherein R₅ is selected from H, alkyl, heteroalkyl, alkenyl, alkynyl, cycloalkyl, aryl, acyl and aroyl; R₆ and R₇ are independently selected from the group consisting of H, alkyl, heteroalkyl, alkenyl, alkynyl, cycloalkyl, aryl, acyl and aroyl, wherein R₆ and R₇ optionally may combine to form a saturated or unsaturated carbocyclic or heterocyclic ring, optionally substituted with one or more Rₐ and R_{b} groups; Y is a bond, or a divalent radical selected from the group consisting of —O—, —S—, —N(R₅)—, C₁-C₄ alkylene, (Z)- or (E)-ethylene, and cycloalkylene with 3 to 6 carbon atoms; Rₐ and R_{b} are independently selected from the group consisting of H, alkyl, heteroalkyl, alkenyl, alkynyl, cycloalkyl, aryl, substituted aryl, arylalkyl, substituted arylalkyl, ketoalkyl, hydroxyalkyl, aminoalkyl, —CH₂NR₃R₄, alkoxy, aryloxy, arylalkyloxy, halo, haloalkyl, cyano, hydroxy, nitro, amino, —NR₃R₄, —SR₅, carboxamido, —C(O)NR₃R₄, —O-carboxamido, —OC(O)NR₃R₄, sulfonamido, and —SO₂NR₃R₄, wherein R₃ and R₄ are selected from H, alkyl, alkenyl, alkynyl, cycloalkyl, hydroxyalkyl and iminomethylamino and wherein optionally R₃ and R₄ together with the N atom to which they are attached to form a 5-7 membered cyclic ring; and
wherein R₁ and R₂ are independently selected from the group consisting of H, substituted or unsubstituted alkyl, substituted or unsubstituted heteroalkyl, substituted or unsubstituted cycloalkyl, and substituted or unsubstituted cycloheteroalkyl, and substituted or unsubstituted phenyl, and substituted or unsubstituted aryl or heteroaryl, and wherein optionally, when X is N, if taken together with the N atom to which they are attached, R₁ and R₂, form a substituted or unsubstituted N-heterocycle or substituted or unsubstituted heteroaryl with the atom to which they are each attached.

62. The use of claim 61 wherein the compound inhibits human brain MGL with an IC₅₀ of less than 1 micromolar.

63. The use of claim 61, wherein the compound is 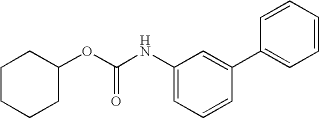
or a pharmaceutically acceptable salt thereof.
